(19) United States
(12) Patent Application Publication
(10) Pub. No.: US 2007/0203325 A1
(43) Pub. Date: Aug. 30, 2007

(54) Integrase-dirived HIV-inhibiting agents

(76) Inventors: Xiaojian Yao, Winnipeg (CA); Zhu-jun Ao, Winnipeg (CA)

Correspondence Address: SMART & BIGGAR;P.O. BOX 2999, STATION D, 900-55 METCALFE STREET, OTTAWA, ON, K1P5Y6, US

(21) Appl. No.: 11/606,904

(22) Filed: Dec. 1, 2006

Related U.S. Application Data
(60) Provisional application No. 60/776,202, filed on Feb. 24, 2006.

(51) Int. Cl. A61K 39/12; C07K 14/00; C07K 16/00; A61K 38/00; C07K 17/00; C07K 2/00; C07K 4/00; C07K 5/00; C07K 7/00

(52) U.S. Cl. 530/300; 424/204.1

(57) ABSTRACT

The present invention relates to agents based on integrase of HIV-1, for inhibiting the proliferation of HIV-1. The agents are derived from the C-terminal domain of HIV-1 integrase, comprising at least one of the regions identified as being important for interaction between integrase and imp7 or impβ, and/or for nuclear localization of the HIV PIC, replication of HIV, or infection of HIV.

### REFERENCE TO RELATED APPLICATIONS

This patent application claims the benefit of U.S. Provisional Application No. 60/776,202, filed Feb. 24, 2006, the content of which is herein incorporated by reference.

### FIELD OF INVENTION

The present invention relates to agents based on integrase of HIV-1, for inhibiting the proliferation of HIV-1.

### BACKGROUND OF THE INVENTION

The integrase (IN) of human immunodeficiency virus type 1 (HIV-1) mediates the integration process. It is also implicated in different steps during viral life cycle including reverse transcription and viral DNA nuclear import.

HIV-1 integrase is encoded by the pol gene. During early phase of the HIV-1 replication cycle, after virus entry into target cells, another pol gene product, reverse transcriptase (RT), copies viral genomic RNA into double-stranded cDNA which exists within a nucleoprotein preintegration complex (PIC). The PIC also contains viral proteins including RT, IN, nucleocapsid (NC, p9), Vpr and matrix (MA, p17) and this large nucleoprotein complex is capable of actively translocating into the cell nucleus, including that of non-dividing cells. This feature is particularly important for the establishment of HIV-1 replication and pathogenesis in exposed hosts, since the infection of postmitotic cells including tissue macrophages, mucosal dendritic cells as well as non-dividing T cells may be essential not only for viral transmission and dissemination, but also for the establishment of persistent viral reservoirs.

HIV-1 IN is composed of three functional domains, an N-terminal domain, a central catalytic core domain and a C-terminal domain, all of which are required for a complete integration reaction. The N-terminal domain harbors an HHCC-type zinc binding domain and is implicated in the multimerization of the protein and contributes to the specific recognition of DNA ends. The core domain of IN contains the highly conserved DDE. The C-terminal domain was shown to possess nonspecific DNA binding properties. Fassati et al EMBO J. 2003 Jul. 15; 22(14): 3675-3685 described nuclear import assays in primary macrophages using purified HIV-1 reverse transcription complexes (RTCs) as substrate. Fassati found that imp7 is a mediator of HIV-1 nuclear import, that small interfering RNA (siRNA) mediated depletion of imp7 in cultured cells, and that recombinant IN could pull down imp7, impα, impβ and transportin from HeLa cell lysates. Fassati also concluded that impβ alone was insufficient to sustain significant RTC nuclear import, and that functional imp7 was necessary.

### SUMMARY OF THE INVENTION

The present invention provides an isolated peptide comprising an IN-derived sequence. The IN-derived sequence comes from the C-terminal domain of integrase, which is generally defined by residues 205 to 288 of the HIV-1 integrase shown by example as SEQ ID NO:1. The IN-derived portion of the peptide has a length of at least 8 amino acids and no more than 83 amino acids that come from the C-terminal domain of integrase. The IN-derived portion of the peptide comprises at least one of the sequences: KELQKQITK (#211-219 of SEQ ID NO:1), KGPAKLLWK (#236-244 SEQ ID NO:1), WKGPAKLLWKGEGAVV (#235-250 SEQ ID NO:1), VVPRRKAK (#259-266 SEQ ID NO:1), KVVPRRKAK (#258-266 SEQ ID NO:1), and PRRKAKII (#261-268 SEQ ID NO:1).

In one form, in the peptide above, the at least 8 and no more than 83 consecutive amino acids of integrase comprises at least one of the sequences: TKELQKQITKLQNFRV (SEQ ID NO:10), PLWKGPAKLLWKGEGAVV (SEQ ID NO:11), PRRKAKIIRDYGK (SEQ ID NO:12), KELQKQITKLQNFRVYYRDSRDPLWKGPAKLLWKG (SEQ ID NO:13), KGPAKLLWKGEGAVVIQDNSDIKVVPRRKAK (SEQ ID NO:14), and KELQKQITKLQNFRVYYRDSRDPLWKGPAKLLWKGEGAVVIQDNSDIKVVPRRKAK (SEQ ID NO:15).

In another form, in the peptide above, the at least 8 and no more than 83 consecutive amino acids of integrase comprises the sequence KELQKQITK (#211-219 of SEQ ID NO:1) or KGPAKLLWK (#236-244 SEQ ID NO:1) or both.

In another form, in the peptide above, the at least 8 and no more than 83 consecutive amino acids of integrase comprises the sequence KGPAKLLWK (#236-244 SEQ ID NO:1) or VVPRRKAK (#259-266 SEQ ID NO:1) or both.

In another form, in the peptide above, the at least 8 and no more than 83 consecutive amino acids of integrase comprises the sequence KELQKQITK (#211-219 of SEQ ID NO:1), KGPAKLLWK (#236-244 SEQ ID NO:1), and VVPRRKAK (#259-266 SEQ ID NO:1).

In another form, in any of the peptides above, the peptide comprises at least 13 and no more than 83 consecutive amino acids from residues 205 to 288 of an HIV-1 integrase sequence, and the at least 13 and no more than 83 consecutive amino acids of integrase comprises at least one of the sequences: KELQKQITK (#211-219 of SEQ ID NO:1), KGPAKLLWK (#236-244 of SEQ ID NO:1), WKGPAKLLWKGEGAVV (#235-250 of SEQ ID NO:1), VVPRRKAK (#259-266 of SEQ ID NO:1), KVVPRRKAK (#258-266 of SEQ ID NO:1), and PRRKAKII (#261-268 of SEQ ID NO:1).

In another form, in any of the peptides above, the IN-derived portion of the peptide has a length of at least 10 amino acids and no more than 83 amino acids that come from the C-terminal domain of integrase. Specifically contemplated are peptides where the IN-derived portion has a length of at least 10, 12, 14, 18, 22, 28, 34, 40, 50, 60, 70 and 80 amino acids that come from the C-terminal domain of integrase. Also contemplated are isolated peptides that consist of IN-derived sequences having a length of at least 10 amino acids and no more than 83 amino acids from the C-terminal domain of integrase (defined by amino acids 205-288 of SEQ ID NO:1 in one embodiment).

In another form, in any of the peptides above, the peptide further comprises a heterologous sequence which is fused with the IN-derived sequence (i.e. derived from residues 205 to 288 of HIV-1 integrase). The heterologous sequence may be a membrane-translocating sequence, e.g. the HIV Tat membrane-translocating sequence (SEQ ID NO:9). The heterologous sequence may also be a reporter sequence.

In another form, any of the peptides above, when expressed with HIV-1 provirus, renders HIV-1 replication-defective or infection-defective.

Another aspect of the invention relates to a variant polypeptide of HIV-1 integrase having a substitution or deletion in at least one of the following positions of HIV-1 integrase: K211, K215, K219, K236, K240, K244, V249, V250, K258, R262, R263, K264, K266, and K273.

Another aspect of the invention relates to a variant polypeptide of HIV-1 integrase having at least one of the following regions deleted: KELQKQITK (#211-219 of SEQ ID NO:1), KGPAKLLWK (#236-244 of SEQ ID NO:1), WKGPAKLLWKGEGAVV (#235-250 of SEQ ID NO:1), VVPRRKAK (#259-266 of SEQ ID NO:1), KVVPRRKAK (#258-266 of SEQ ID NO:1), and PRRKAKII (#261-268 of SEQ ID NO:1), or having a combination of the above deletions and substitutions where the substitutions occur in at least one of the following positions of HIV-1 integrase: K211, K215, K219, K236, K240, K244, V249, V250, K258, R262, R263, K264, K266, and K273. In one embodiment, the variant polypeptide comprises residues 205 to 288 of HIV-1 integrase. (This takes into account the positions and regions other than those substituted or deleted.)

In other embodiments, the variant polypeptide is a truncated version of integrase containing the substitutions and/or deletions described above. The truncated variant has an N-terminus corresponding to any position of integrase from amino acid 1 to amino acid 205 and has a C-terminus corresponding to any position of integrase from amino acid 267 to 288.

In other embodiments, the variant polypeptide is a truncated version of integrase that lacks at least one region defined by amino acids 212-288, 240-288, 258-288, 212-266.

Another aspect of the invention relates to a variant polypeptide described herein that, when expressed with HIV-1 provirus, renders HIV-1 replication-defective or infection-defective.

The variant polypeptide may have impaired binding to imp7 or impβ.

Another aspect of the invention relates to a fusion polypeptide comprising the variant polypeptide described herein fused to a heterologous sequence. The heterologous sequence may be a membrane-translocating sequence such as the HIV Tat membrane-translocating sequence (SEQ ID NO:9).

Another aspect of the invention relates to an isolated polynucleotide encoding any of the peptide or variant polypeptide or fusion polypeptide described above.

Another aspect of the invention relates to a monoclonal antibody or fragments thereof specifically immunoreactive against at least one of the sequences: KELQKQITK (#211-219 of SEQ ID NO:1), KGPAKLLWK (#236-244 of SEQ ID NO:1), WKGPAKLLWKGEGAVV (#235-250 of SEQ ID NO:1), VVPRRKAK (#259-266 of SEQ ID NO:1), KVVPRRKAK (#258-266 of SEQ ID NO:1), PRRKAKII (#261-268 of SEQ ID NO:1), TKELQKQITKLQNFRV (SEQ ID NO:10), PLWKGPAKLLWKGEGAVV (SEQ ID NO:11), PRRKAKIIRDYGK (SEQ ID NO:12), KELQKQITKLQNFRVYYRDSRDPLWKGPAKLLWKG (SEQ ID NO:13), KGPAKLLWKGEGAVVIQDNSDIKVVPRRKAK (SEQ ID NO:14), and KELQKQITKLQNFRVYYRDSRDPLWKGPAKLLWKGEGAVVIQDNSDIKVVPRRKAK (SEQ ID NO:15). The monoclonal antibody may be a single chain monoclonal antibody. The monoclonal antibody may inhibit binding of HIV-1 integrase with imp7 or impβ. The monoclonal antibody fragment or single chain monoclonal antibody may be fused with a heterologous sequence which may be a membrane-translocating sequence, e.g. the HIV Tat membrane-translocating sequence.

Another aspect of the invention relates to a chemically synthesized double stranded short interfering nucleic acid (siNA) molecule that directs cleavage via RNA interference (RNAi) of a HIV RNA encoding amino acids 205 to 288 of HIV-1 integrase, wherein a) each strand of said siNA molecule is about 18 to about 23 nucleotides in length; and b) one strand of said siNA molecule comprises nucleotide sequence having sufficient complementarity to said HIV RNA for the siNA molecule to direct cleavage of the HIV RNA via RNA interference. Each strand of the siNA molecule may be about 18 to about 23 nucleotides in length; and one strand of the siNA molecule may comprise a nucleotide sequence having sufficient complementarity to SEQ ID NO:16 for the siNA molecule to direct cleavage of the HIV RNA via RNA interference. In one embodiment, each strand of said siNA molecule is about 21 nucleotides in length.

Another aspect of the invention relates to a method of inhibiting HIV-1 replication in a cell, comprising transporting into the cell any of the peptide, the variant polypeptide, the fusion polypeptide, the monoclonal antibody, or the short interfering nucleic acid (siNA) molecule described herein.

Another aspect of the invention relates to a method of inhibiting HIV-1 replication in a cell, comprising expressing in the cell any of the peptide, the variant-polypeptide, the fusion polypeptide, the monoclonal antibody, or the short interfering nucleic acid (siNA) molecule described herein.

Another aspect of the invention relates to a method of inhibiting HIV-1 infection in a human comprising administering to the human the peptide, the variant polypeptide, the fusion polypeptide, the monoclonal antibody, or the short interfering nucleic acid (siNA) molecule described herein.

Another aspect of the invention relates to a method for screening for a compound that affects HIV-1 replication or infection, the method comprising: (a) incubating, in the presence of a candidate agent, the IN-derived peptide as defined herein with imp7 or impβ, under conditions suitable for binding to occur between the peptide and imp7 or impβ; (b) determining the level of binding between the peptide and imp7 or impβ, wherein detecting a change in the level of binding between the peptide and imp7 or impβ in the presence of the candidate agent, compared to the level of binding in the absence of the candidate agent, indicates that said agent is a compound that affects HIV-1 replication or infection. The screening method may screen for a compound that inhibits HIV-1 replication or infection, which would require detecting a decrease in the level of binding between the peptide and imp7 or impβ in the presence of the candidate agent, compared to the level of binding in the absence of the candidate agent.

Another aspect of the invention relates to a method for screening for a compound that affects HIV-1 replication or infection, the method comprising: (a) providing a cell that expresses (i) the IN-derived peptide as defined herein and (ii) imp7 or impβ; (b) providing the cell with a candidate agent; and (c) determining the level of binding between the expressed peptide and the expressed imp7 or impβ, wherein detecting a change in the level of binding between the peptide and imp7 or impβ in the presence of the candidate agent, compared to the level of binding in the absence of the candidate agent, indicates that said agent is a compound that affects HIV-1 replication or infection. The screening method may screen for a compound that inhibits HIV-1 replication or infection, which would require detecting a decrease in the level of binding between the peptide and imp7 or impβ in the presence of the candidate agent, compared to the level of binding in the absence of the candidate agent.

### BRIEF DESCRIPTION OF THE DRAWINGS

The present invention will be further understood from the following description with reference to the drawings, in which:

FIG. 1 shows the interaction of HIV-1 IN and importin 7.

1A) Schematic representation of constructs of IN-YFP, T7-imp7 and T7-imp8. For IN-YFP, a full-length wild-type HIV-1 IN was fused in frame to the N-terminus of EYFP. For T7-imp7 and imp8, a T7-tag (9 amino acids) was fused in frame to the N-terminus of imp7 and imp8.

1B) Expression of IN-YFP and T7-imp7 and T7-imp8. Cell lysates from about 6×10⁵ 293T cells transfected with CMV-YFP, CMV-IN-YFP or indicated importin expressors was analyzed with immunoprecipitation with rabbit anti-GFP antibody followed by western blotting using mouse anti-GFP antibody (lanes 1 to 3) or immunoprecipitation with mouse anti-T7 antibody followed by western blotting using the same antibody (lanes 4 to 5).

1C) The in vivo co-IP assay. CMV-IN-YFP was co-transfected with plasmids for T7-imp7 (lane 3) or T7-imp8 (lane 4) into 2×10⁶ 293T cells. As a control, CMV-YFP also was co-transfected with each importin expressing plasmid (lane 1, 2). After 48 hr of transfection, cells were lysed by 0.5% CHAPS buffer and immunoprecipitated with rabbit anti-GFP antibody. Then, immunoprecipitated complexes were resolved by 12.5% SDS-PAGE and immunoblotted with either mouse anti-T7 antibody (upper panel) or mouse anti-GFP antibody (middle panel). The unbound T7-imp7 and T7-imp8 were also checked by sequential immunoprecipitation with anti-T7 antibody followed by immunoblotting with the same antibody (lower panel).

FIG. 2 shows that HIV-1 IN interacts with endogenous imp7 and that the interaction between IN and impβ takes place in the cells.

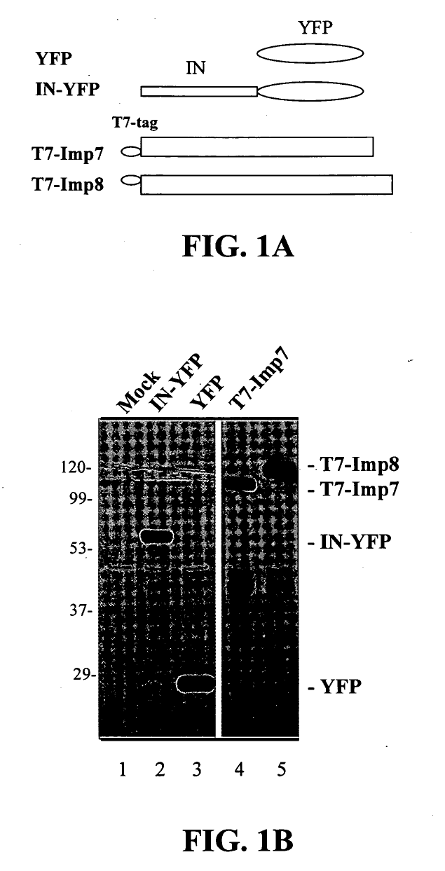

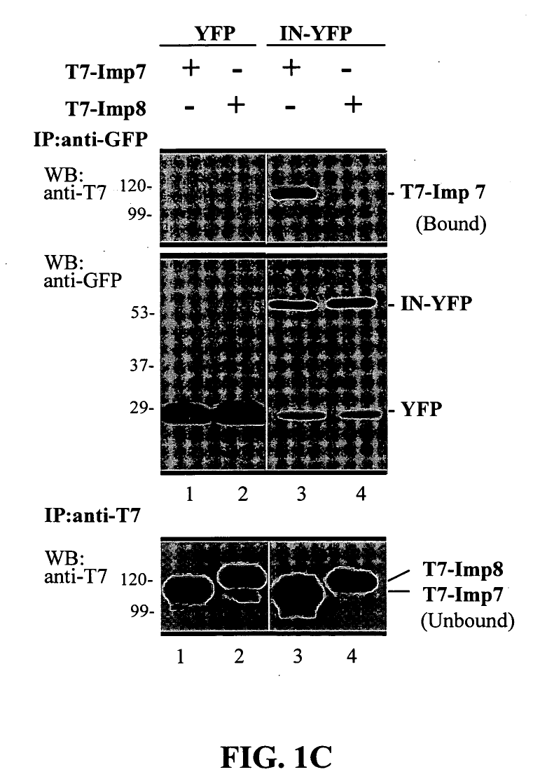

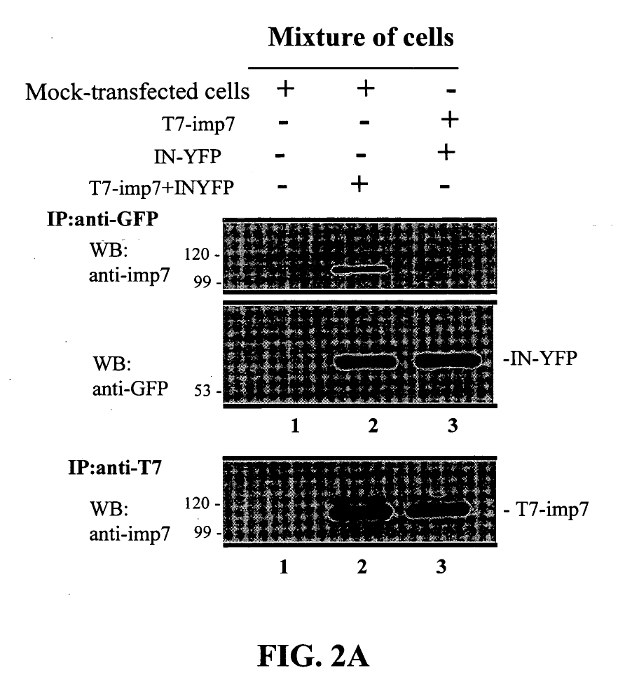

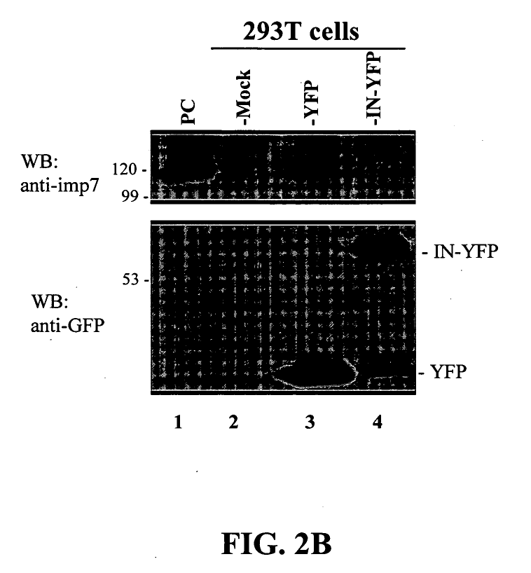

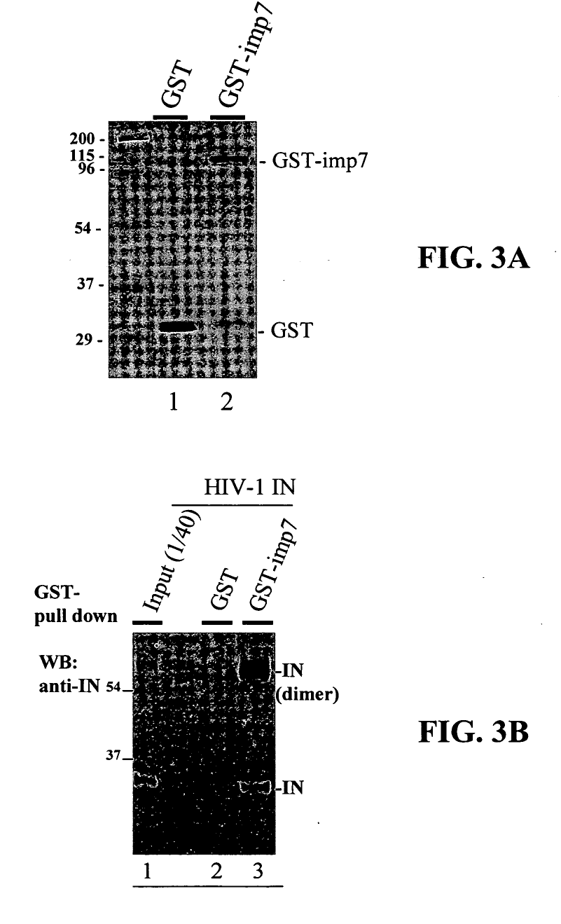

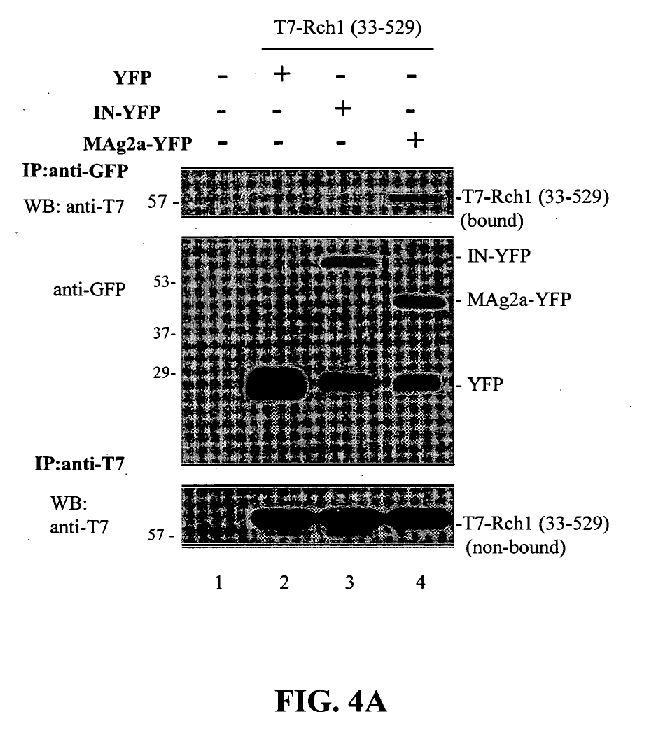

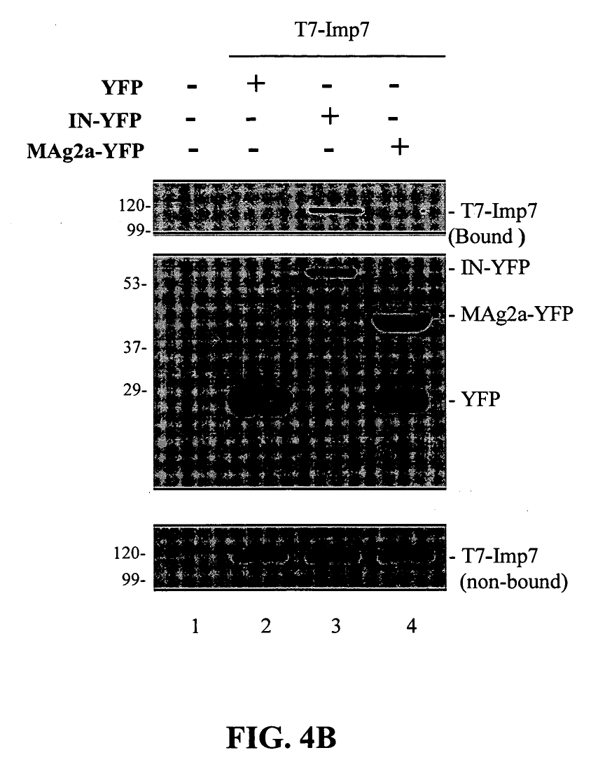

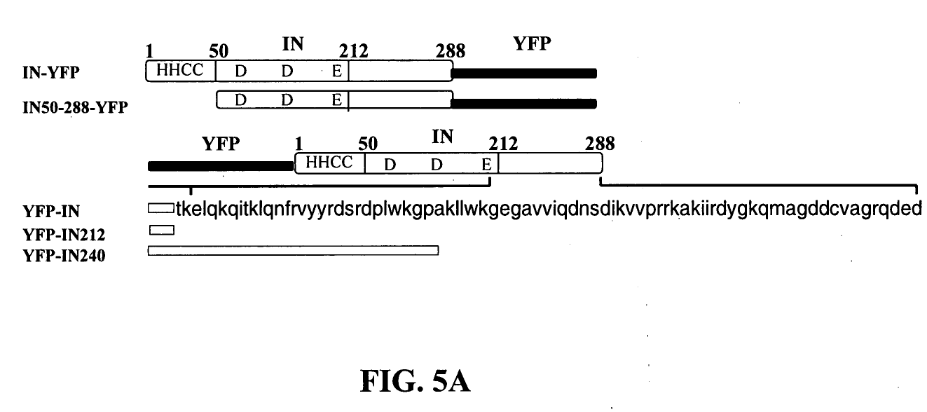

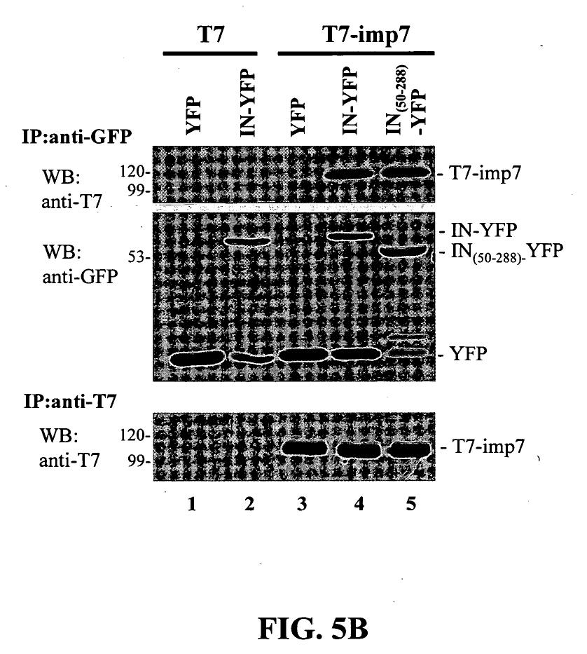

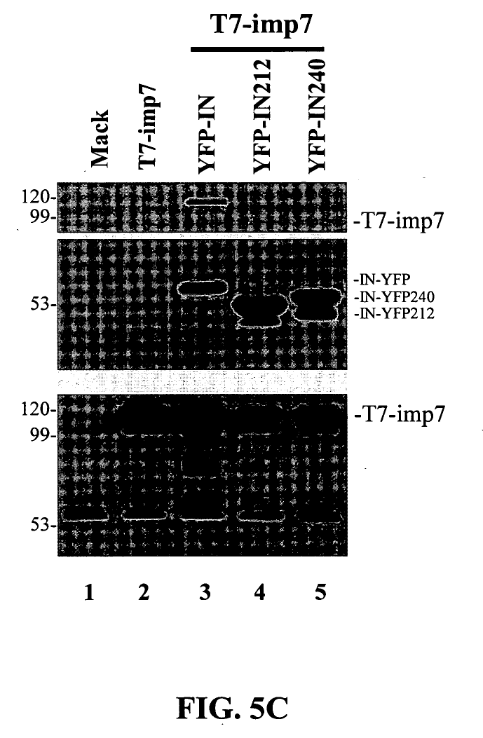

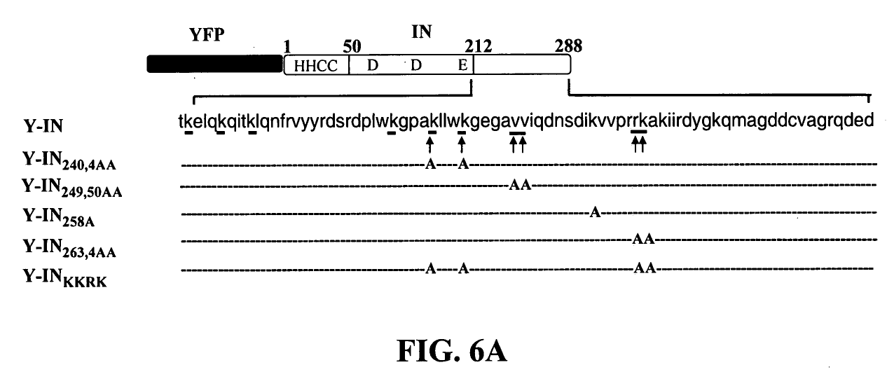

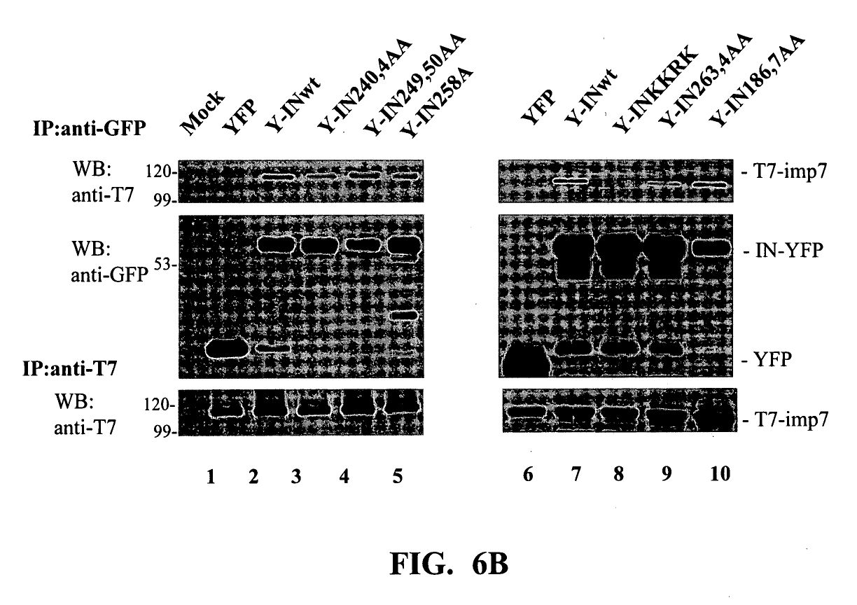

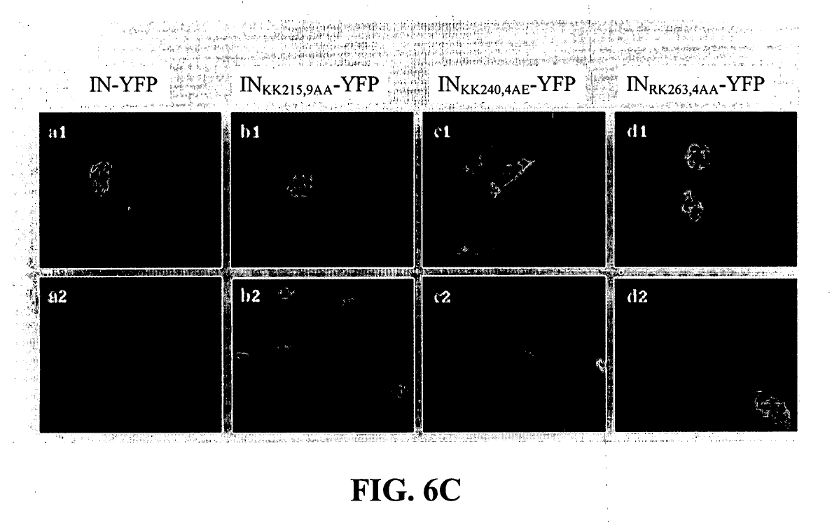

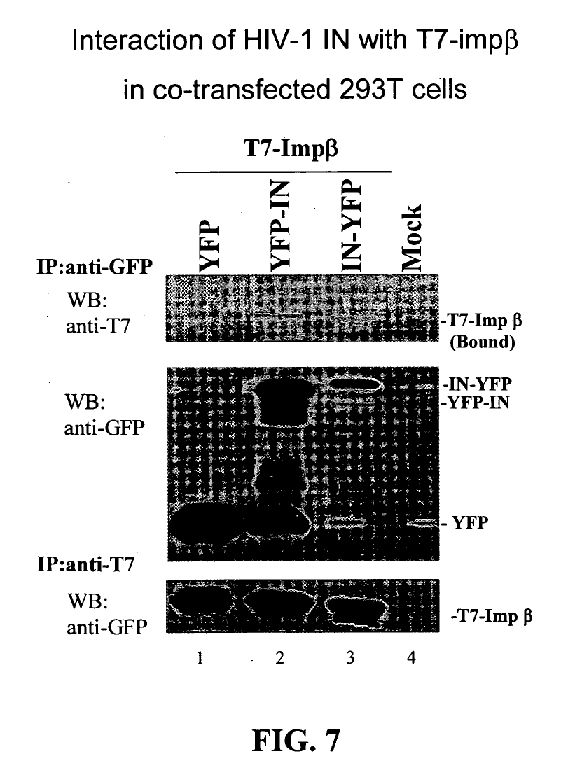

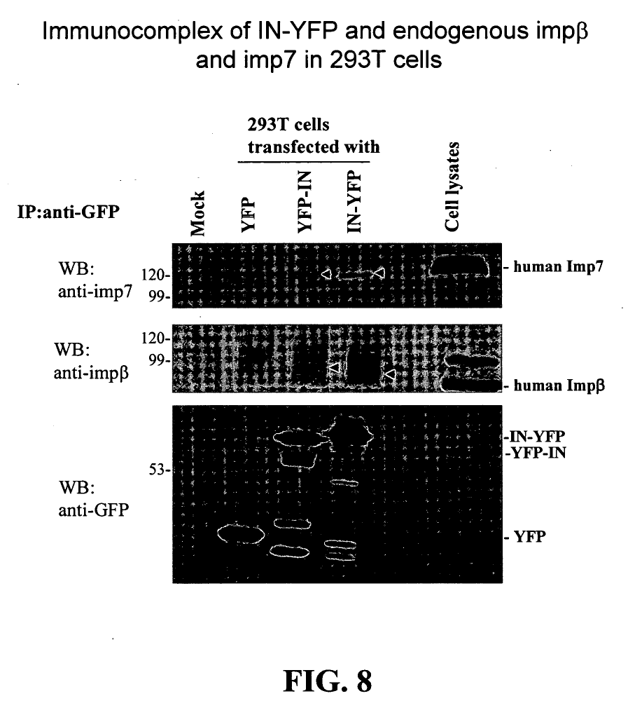

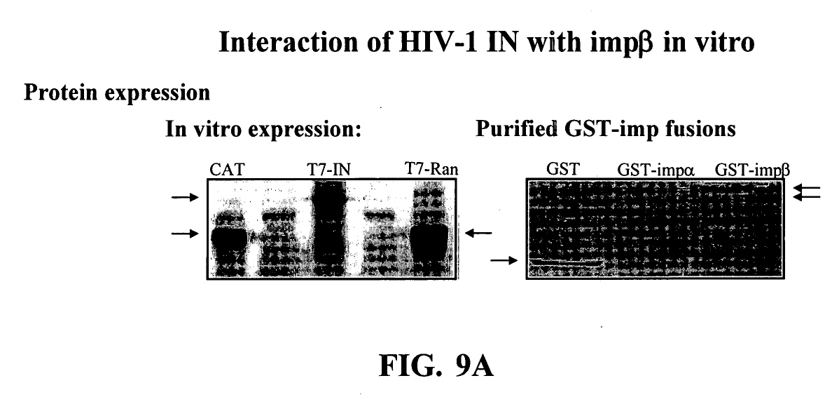

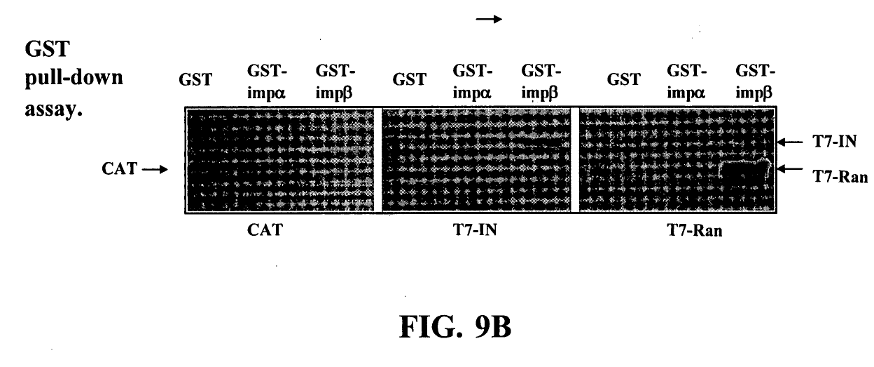

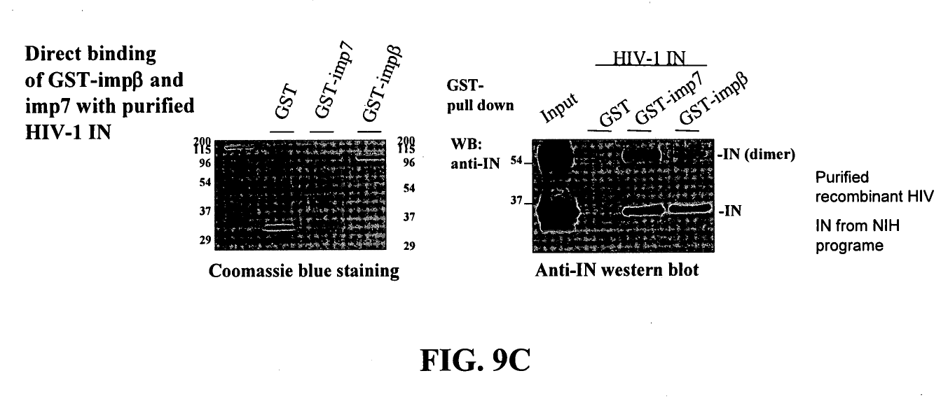

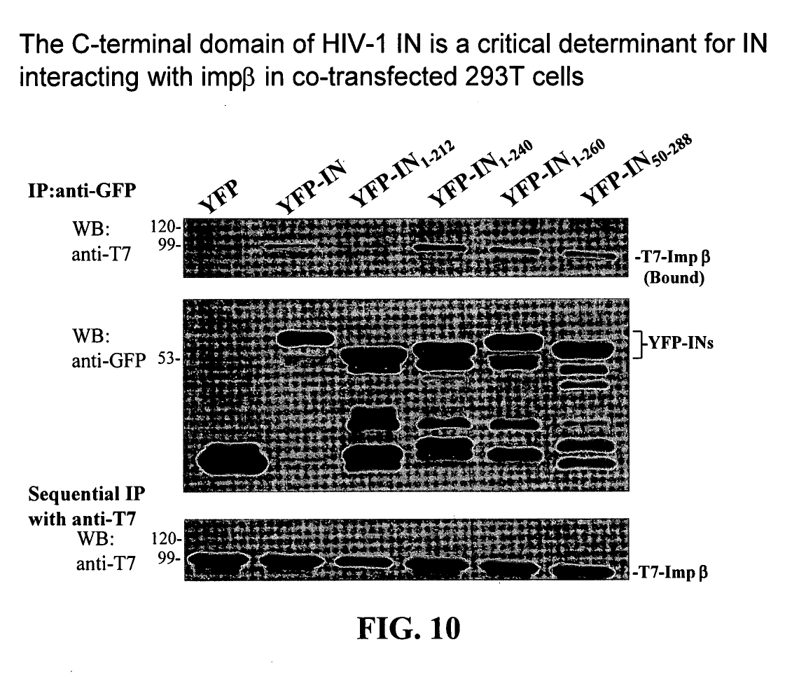

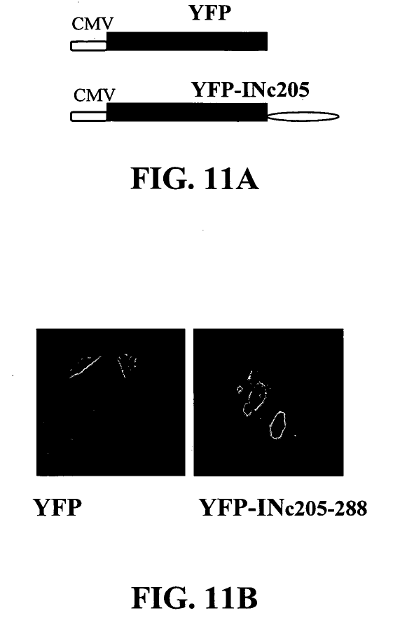

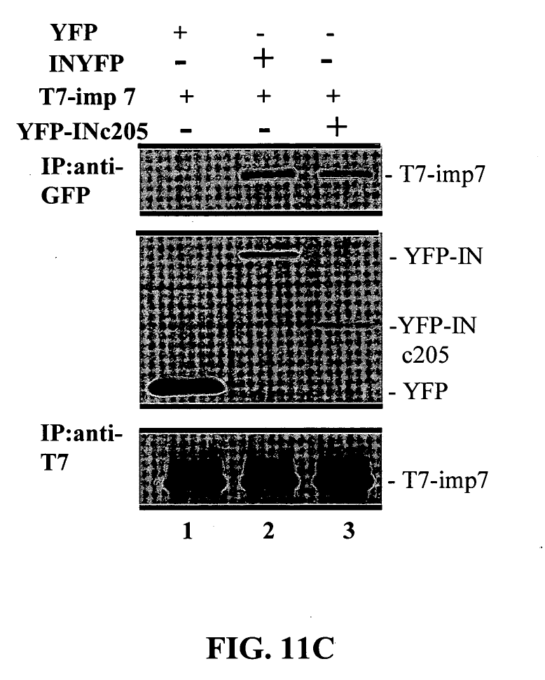

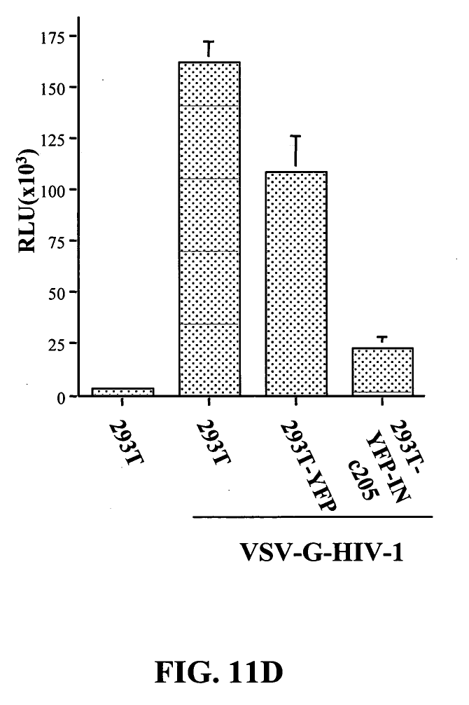

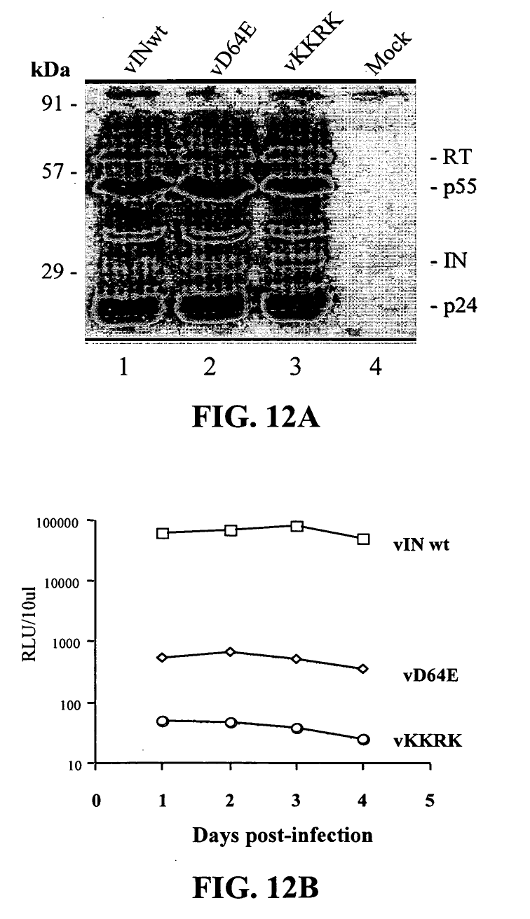

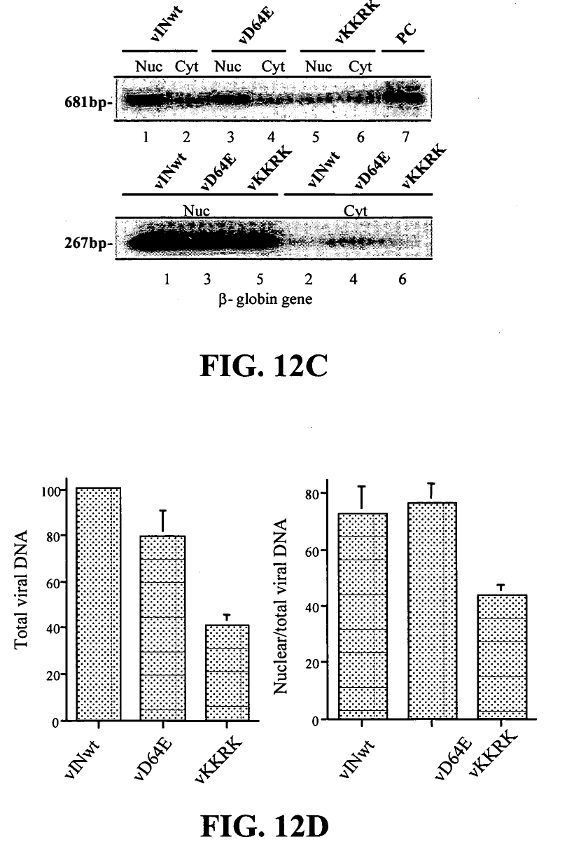

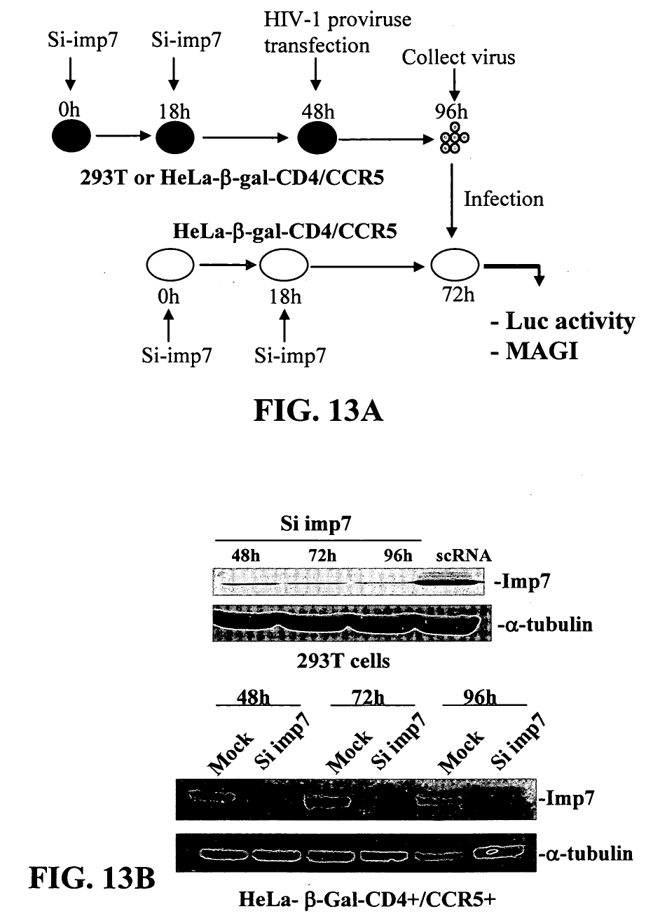

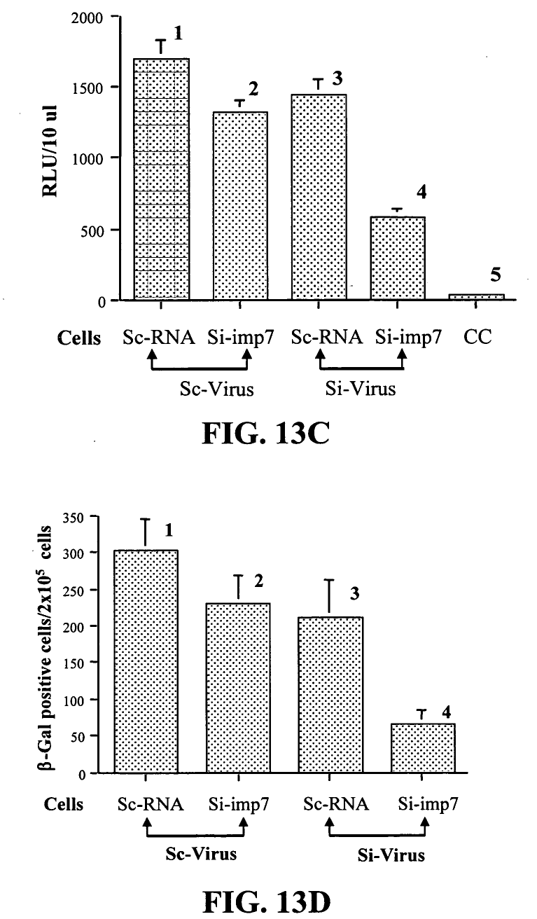

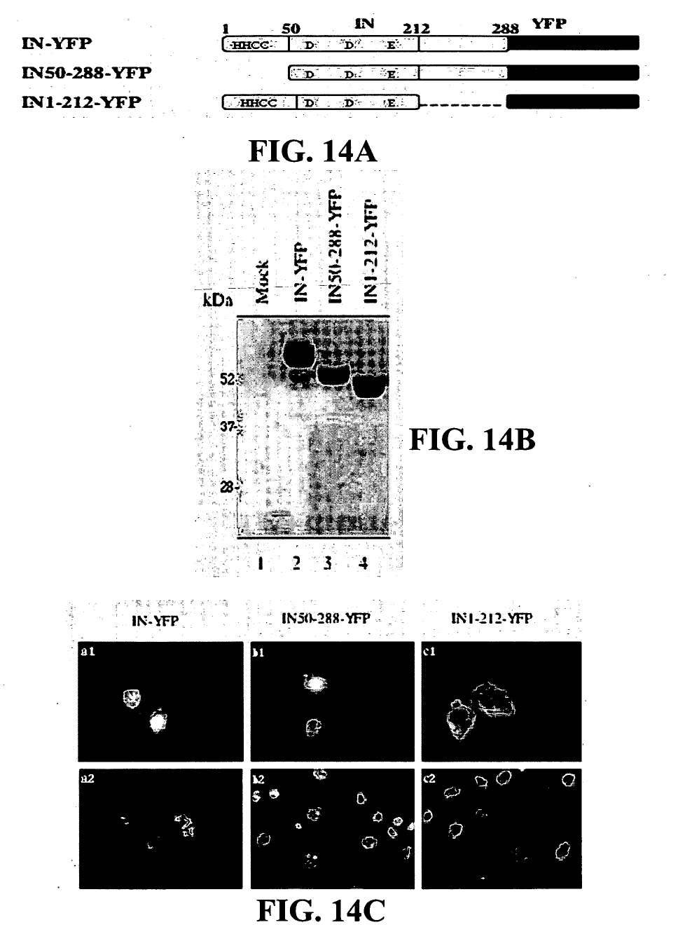

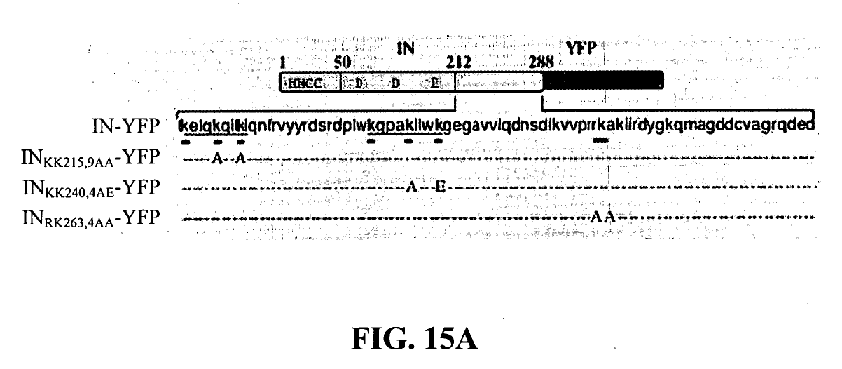

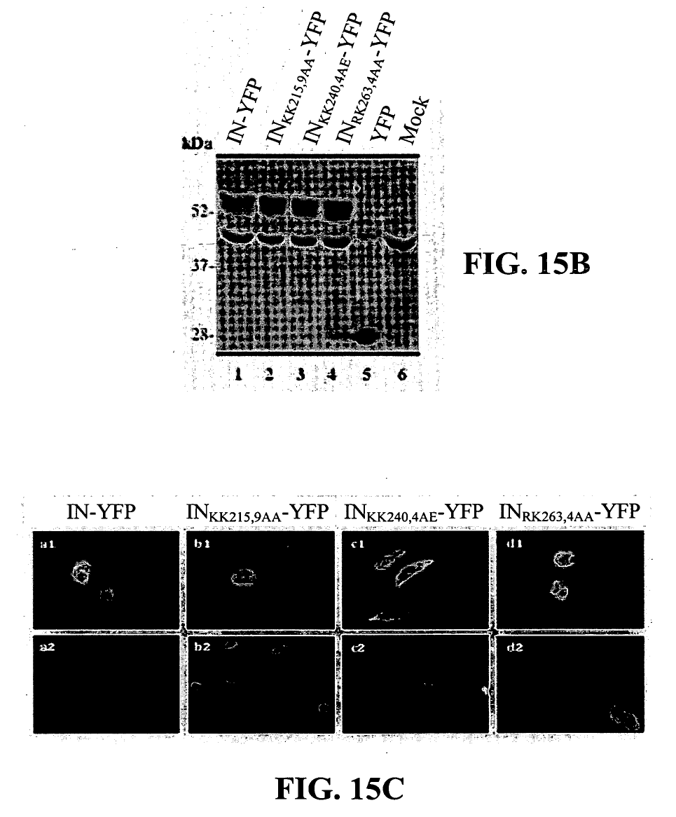

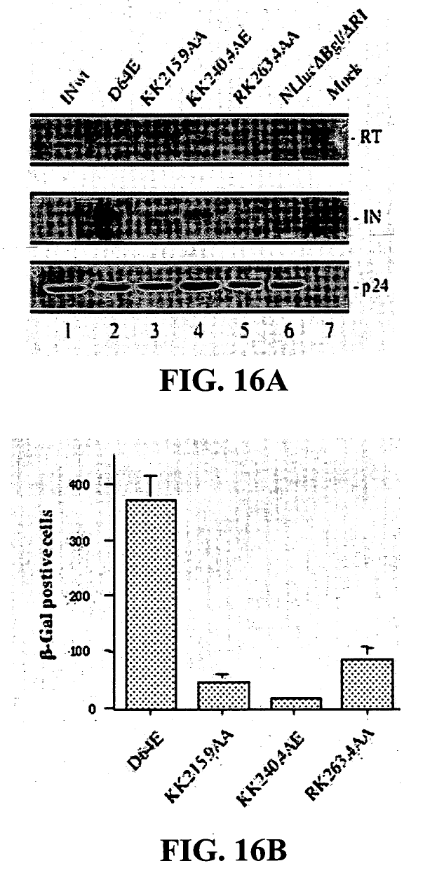

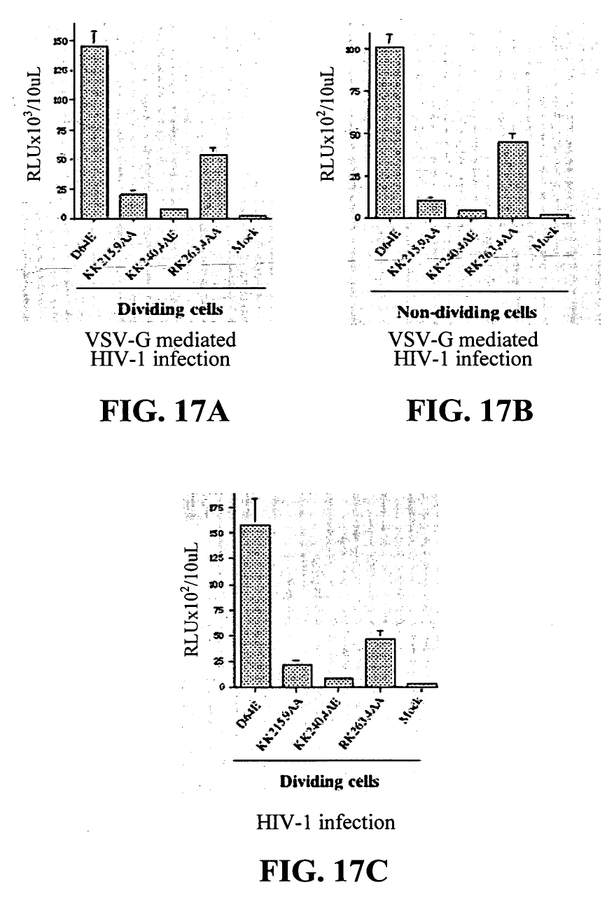

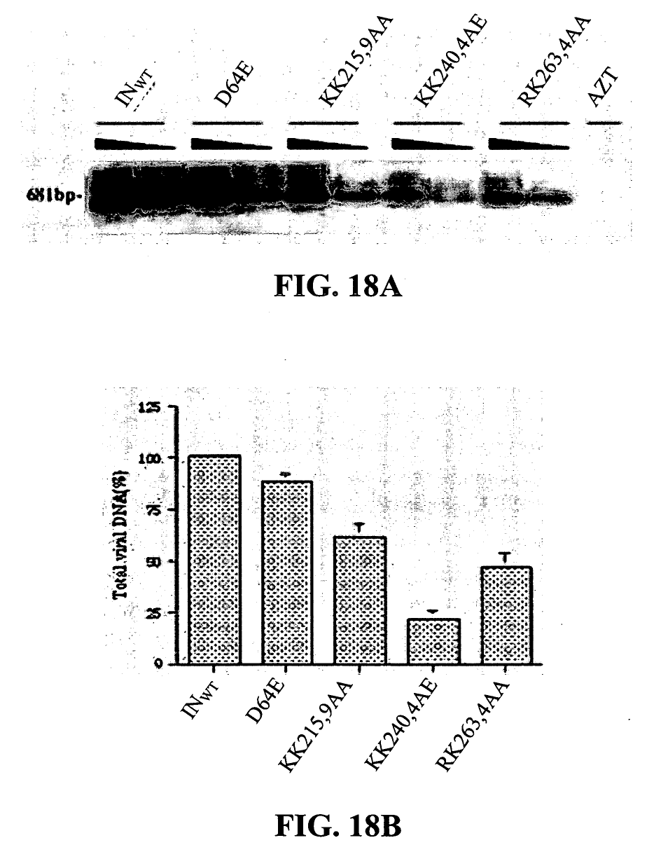

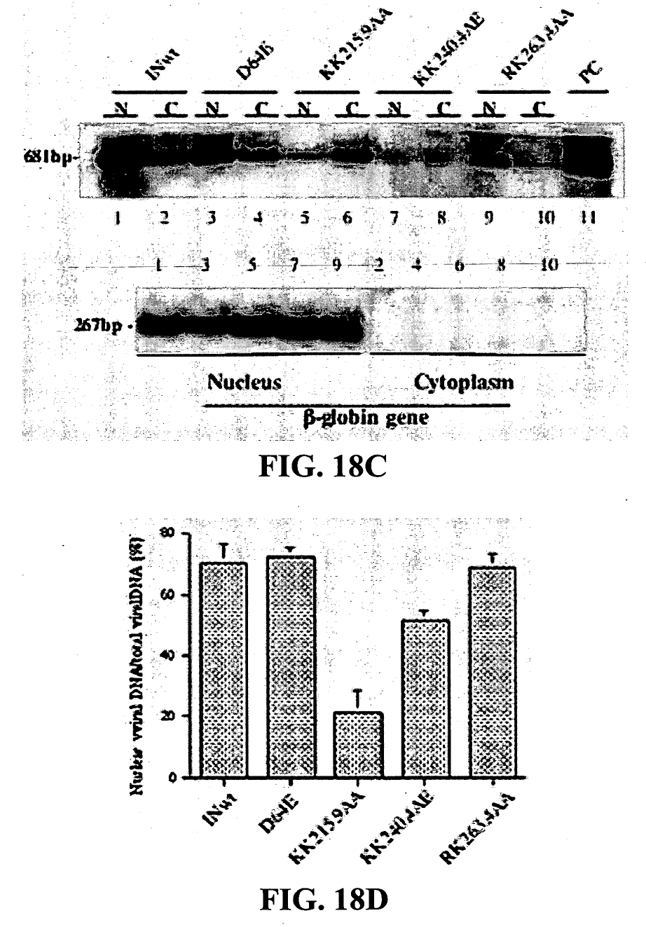

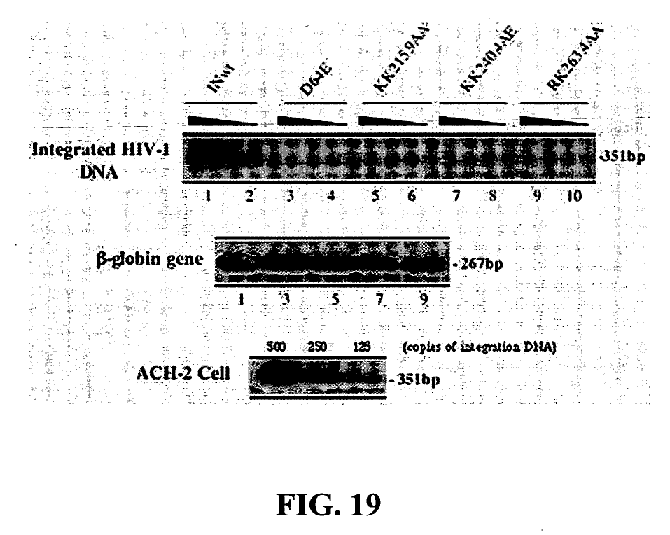

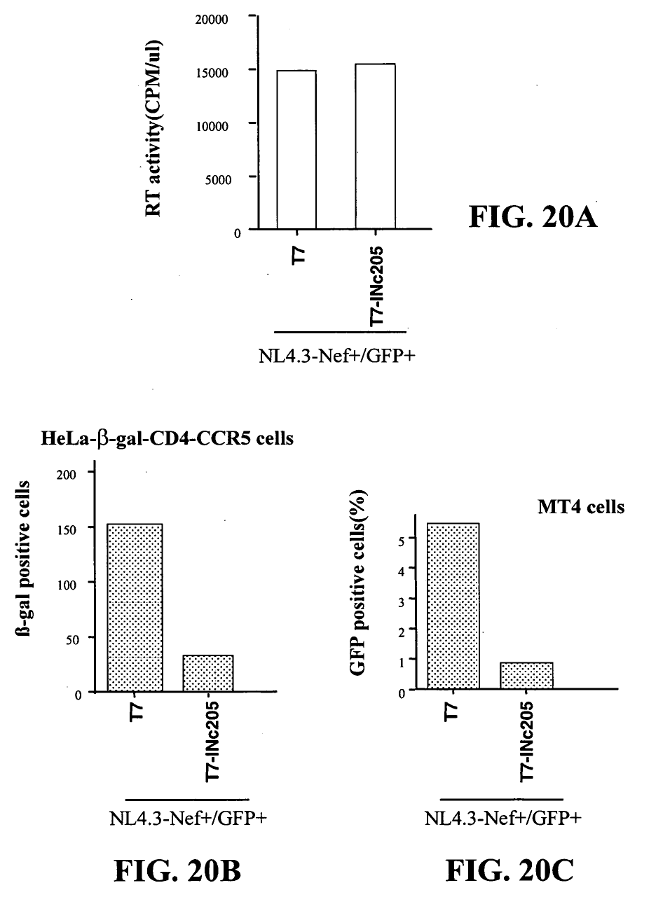

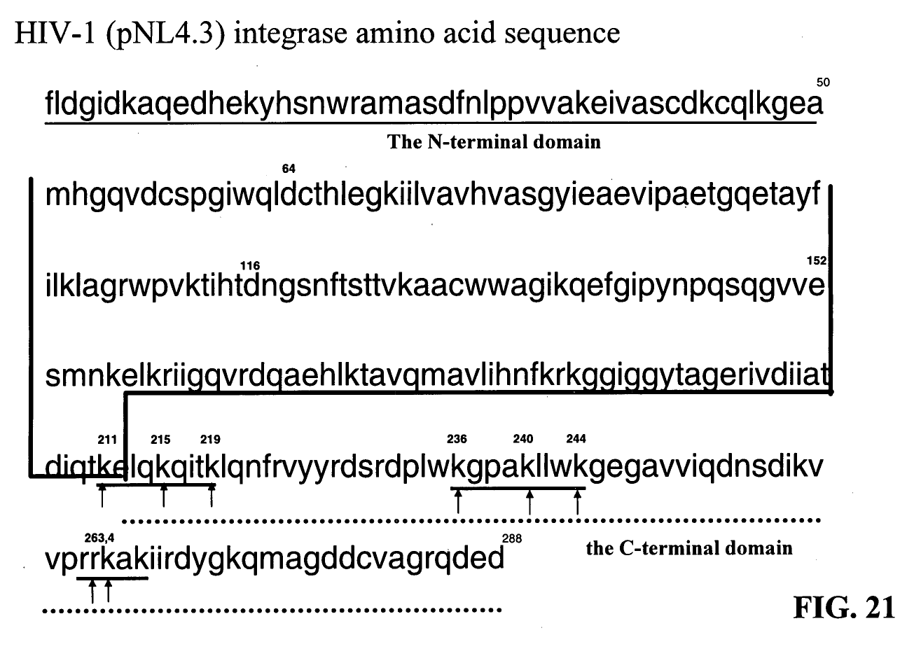

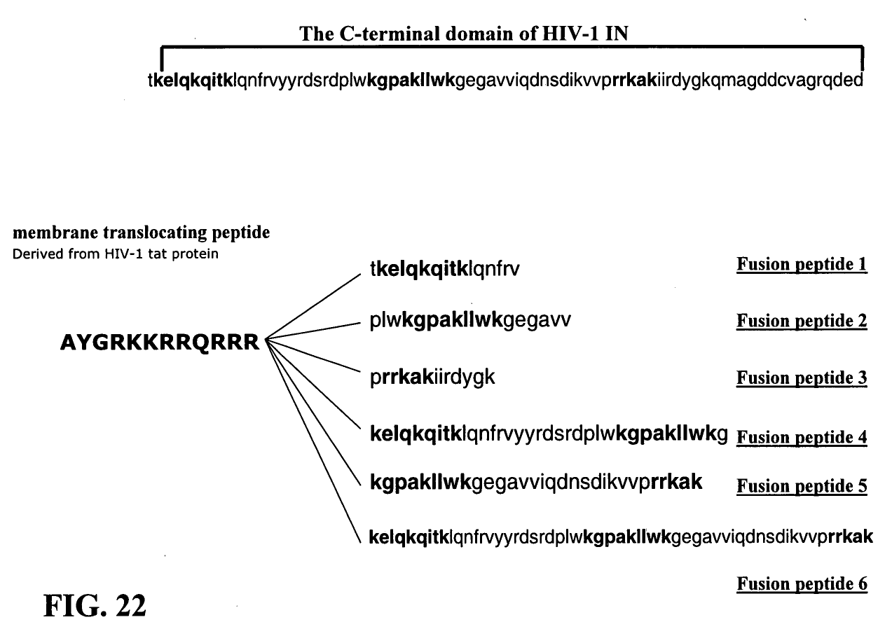

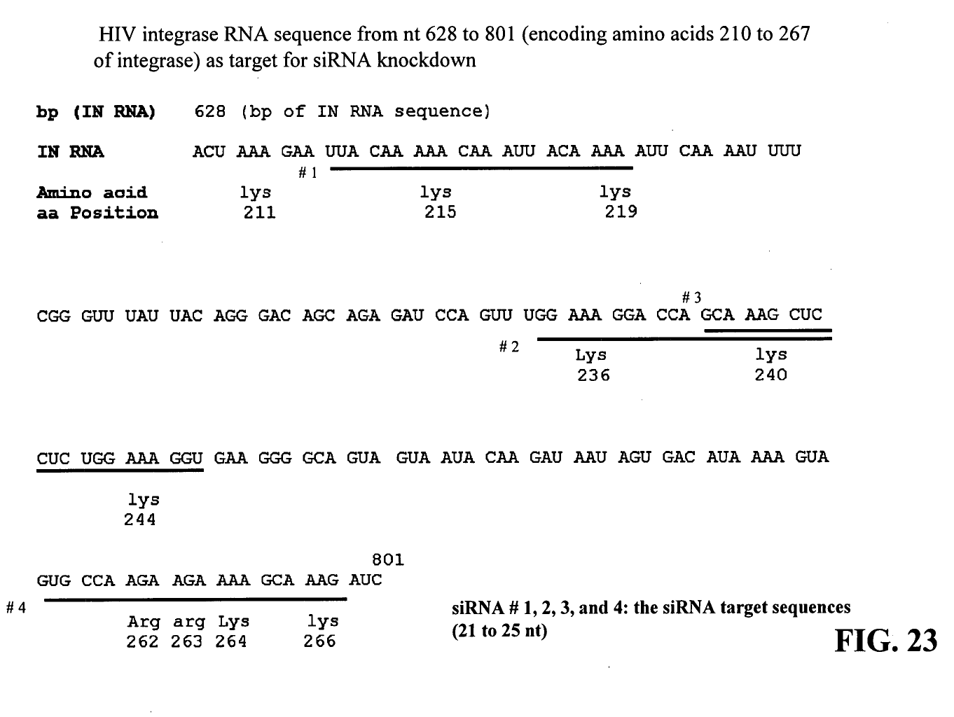

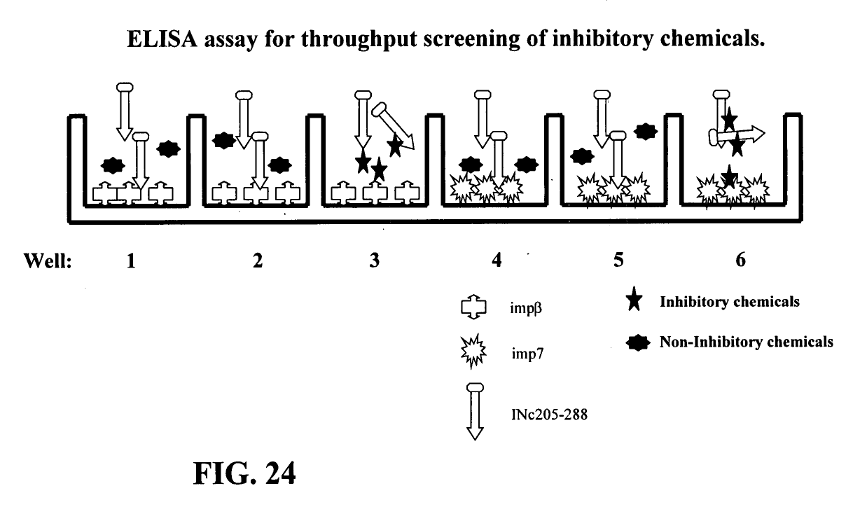

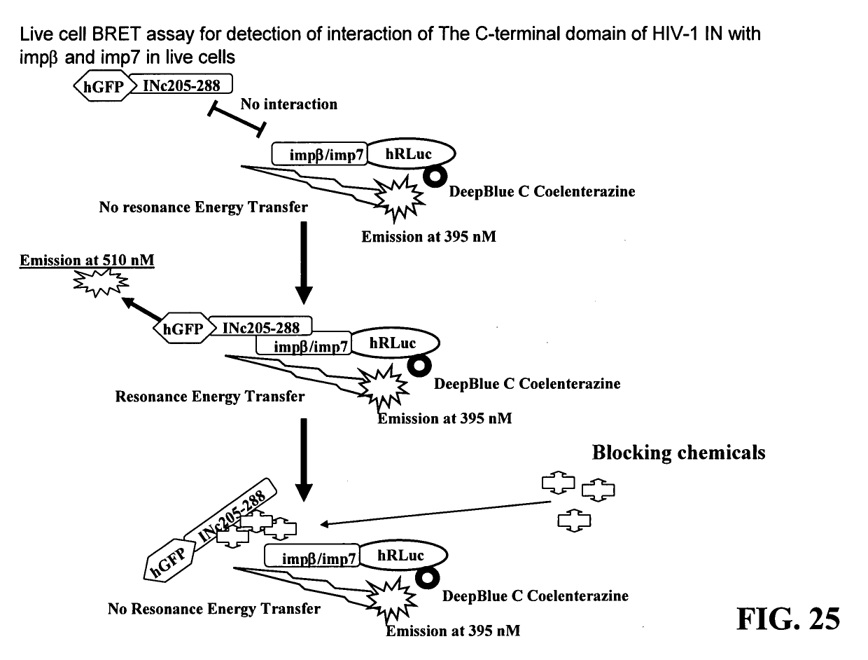

2A) The IN-YFP and T7-imp7 plasmids were co-transfected (lane 2) or transfected individually (lane 3) into 293T cells. After 48 hrs, cells were mixed accordingly, lysed and analyzed with co-immunoprecipitation using the same procedure as FIG. 1C. Upper panel: co-precipitated imp7 detected by western blot with anti-T7 antibody; Middle panel: The expression of IN-YFP detected by western blot with mouse anti-GFP antibody; Lower panel: the unbound imp7 visualized by immunoprecipitation and western blot by anti-T7 antibody.

2B) IN interacts with endogenous imp7. 10×10⁶ 293T cells were mock-transfected (lane 1) or transfected with CMV-YFP (lane 2) and CMV-IN-YFP (lane 3). After 48 hours of transfection, cells were lysed by 05%. CHAPS buffer and immunoprecipitated with rabbit polyclone anti-GFP antibody. Then, immunoprecipitates were separated in 10% SDS-PAGE followed by immunoblotting with rabbit anti-importin 7 (upper panel) or monoclonal anti-GFP antibody (lower panel). In parallel, 2×10⁶ of non-transfected 293T cells were lysed with the same lysis buffer and 10% of celllysates were loaded in SDS-PAGE as positive control (PC).

FIG. 3 shows in vitro interaction between IN and imp7.

3A) GST (lane 1) and GST-imp7 (lane 2) were expressed in E coli and affinity-purified on amylose resin. The similar amount of purified protein was directly loaded on a 12.5% SDS-PAGE followed by the Coomassie Blue staining.

3B) Equal amount of GST (lane 1) and GST-imp7 (lane 2) was incubated with a purified recombinant HIV-1 IN in 199 medium (containing 0.1% CHAPS) for 2 hours at 4° C. Then, the glutathione-sepharose 4B beads were added and incubate for additional one hour. After incubation, the beads were washed five times with the same lysis buffer and the protein complexes bound to glutathione-sepharose 4B beads were eluted with 10 mM glutathione buffer and loaded onto a 12.5% SDS-PAGE followed by western blot analysis with rabbit anti-IN specific antibodies.

FIG. 4 shows differential binding ability of HIV-1 MAp17 and IN to cellular importins Rch1 and imp7.

4A) HIV-I MAp17_{G2A}, but not IN, binds to T7-Rch1. 293T cells were co-transfected by CMV-T7-Rch1 with YFP (lane 2), IN-YFP (lane 3) or MAp17_{G2A}-YFP expressor (lane 4). After 48 hrs of transfection, cells were lysed by CHAPS lysis buffer and immunoprecipitated with rabbit anti-GFP antibody followed by western blot with either anti-T7 or mouse anti-GFP antibodies, as described in the legend for FIG. 1C. Upper panel shows the co-precipitated T7-Rch1 protein. The middle panel shows the expression of YFP, IN-YFP or Map17_{G2A}-YFP and the lower panel reveals the unbound T7-Rch1.

4B) HIV-1 IN, but not MAp17_{G2A}, binds to imp7. 293T cells were co-transfected with YFP (lane 2), IN-YFP (lane 3) or MAp17_{G2A}-YFP (lane 4) plasmid with T7-imp7 expressor. Upper panel indicates the co-precipitated T7-imp7; the middle panel shows the expression of YFP, IN-YFP or MAp17_{G2A}-YFP and the lower panel reveals the unbound T7-imp7 in each cell lysate sample.

FIG. 5 indicates the region(s) of HIV-1 IN that interact with imp7.

5A) Schematic representation of IN-YFP and YFP-IN truncated proteins used for binding assay. The IN sequence shown corresponds to amino acids 210-288 of SEQ ID NO:1.

5B) The N-terminal domain is dispensable for IN:imp7 interaction. The YFP (lane 3), IN-YFP (lane 4) and IN₅₀₋₂₈₈-YFP (lane 5) were co-expressed with T7-imp7 in 293T cells. In parallel, YFP and IN-YFP were expressed alone in 293T cells as control (lanes 1 and 2). At 48 hrs of transfection, cells were lysed and the interaction between IN-YFP mutants and imp7 was analyzed using anti-GFP immunoprecipitation and subsequently western blot with anti-T7 or anti-GFP antibodies, as described in FIG. 1C. The upper panel reveals the co-precipitated T7-imp7 and the middle panel shows the expression of YFP, IN-YFP, IN₅₀₋₂₈₈-YFP, as indicated. The lower panel shows the detection of unbound T7-imp7 by anti-T7 immunoprecipitation and western blot.

5C) The C-terminal domain is required for IN:imp7 interaction. The IN full-length protein (lane 3), IN₁₋₂₁₂ (lane 4), IN₁₋₂₄₀ (lane 5) and IN₁₋₂₆₀ (lane 6) were assayed for the interaction with imp7 as described before. Upper panel: co-precipitated T7-imp7. Middle panel: Expression of YFP, YFP-IN and YFP-IN mutants. Lower panel: unbound T7-imp7.

FIG. 6 shows the effect of different IN C-terminal substitutions on IN:imp7 interaction.

6A) Diagram of HIV-1 IN domain structure and introduced mutations at the C-terminal domain of the protein. The position of introduced mutation is shown at the bottom of sequence. The IN sequence shown corresponds to amino acids 210-288 of SEQ ID NO:1.

6B) Both of KK240,4 and RK263,4 of IN are involved in Imp7 interaction. The YFP (lanes 2 and 7), YFP-INwt (lanes 3 and 8) and different YFP-IN mutant expressors were co-transfected with T7-Imp7 expressor in 293T cells and after 48 h of infection, cells were lysed with CHAPS lysis buffer and the IN/Imp7 interaction for each IN mutant was analyzed by using the same protocol as described in FIG. 1C. Upper panel: co-precipitated T7-Imp7. Middle panel: Expression of YFP, YFP-INwt and YFP-IN mutants. Lower panel: unbound T7-Imp7. The position of each immunoprecipitated and co-precipitated proteins were indicated on the right side of the gel.

6C) The YFP-IN mutants as in B) were transfected into cells and visualized by fluorescence.

FIG. 7 shows interaction of HIV-1 IN with T7-impβ in co-transfected 293T cells. 293T cells were mock-transfected or transfected with SVCMVin-YFP, SVCMVin-IN-YFP or SVCMVin-YFP-IN expressors. Cells were lysed with 199 medium containing 0.25% NP-40 and a protease inhibitor cocktail (Roche), and clarified by centrifugation at 13,000 rpm for 30 min at 4° C.

The supernatant was subjected to immunoprecipitation with rabbit anti-GFP antibody and immunoprecipitates were resolved by 10% SDS-PAGE gel followed by western blot using mouse anti-T7 (upper panel) or mouse anti-GFP antibodies (middle panel), respectively. Also, the total T7-Impβ expression in cell lysates was sequentially immunoprecipitated with mouse anti-T7 antibody followed by western blot using the same antibody (lower panel).

FIG. 8 shows an immunocomplex of IN-YFP and endogenous impβ and imp7 in 293T cells. 293T cells were transfected with SVCMVin-YFP, SVCMVin-IN-YFP or SVCMVin-YFP-IN expressor. After 48 hours of transfection, cells were lysed by 199 medium with 0.25% NP-40 and immuno-precipitated with anti-GFP followed by western blot with a rabbit anti-human Impβ antibody (Cat# SC-11367, Santa Cruz Biotechnology Inc) (shown in middle panel) and anti-GFP antibody (shown in the lower panel). Then, the nitrocellular membrane from the middle panel was stripped with glycine/HCl buffer (0.1M glycine, pH. 2.7) and re-processed with western blot with anti-imp7 antibody (shown in upper panel). The positions of different proteins are shown at the right side of the gel.

FIG. 9 shows interaction of HIV-1 IN with impβ in vitro.

9A) Protein expression. Left panel: [³⁵S]methionine-labeled CAT, T7-IN and T7-Ran protein were expressed in vitro using TnT T7 coupled reticulocyte lysate system, extracts were separated on a SDS-PAGE and expressed proteins were detected by autoradiography. Right panel: Purified GST (Control), GST-Impα, and GST-impβ were verified by directly loading on a 12.5% SDS-PAGE followed by the Coomassie Blue staining.

9B) Impβ interacts with T7-IN and with T7-Ran, but not with T7-CAT. A GST pull-down assay was conducted as described above. Following extensive washing in 199 medium containing 0.25% NP40, the bound protein complexes were eluted with 50 mM glutathione and separation on a SDS-PAGE followed by autoradiography.

9C) Direct binding of GST-impβ and imp7 with purified HIV-1 IN. Left panel: as above, GST, GST imp7 and impβ were expressed and purified. Purified proteins were separated on an SDS-PAGE and detected by Coomassie Blue staining. Right panel: A GST pull-down assay was conducted as described above, with purified HIV-1 IN protein. Following extensive washing in 199 medium containing 0.25% NP40, the bound protein complexes were eluted with 50 mM glutathione and separation on a SDS-PAGE and finally pulled-down protein was detected by a western anti-IN antibody.

FIG. 10 shows that the C-terminal domain of HIV-1 IN interacts with impβ in co-transfected 293T cells. YFP-IN and different mutant expressors, as indicated, were co-transfected with T7-impβ in 293T cells. After 48 hours, cells were lysed, immunopreciptated with anti-GFP followed by western blot with anti-T7 antibody (upper panel) and anti-GFP antibody (middle panel). The total amount of T7-impβ was analyzed by sequential IP with anti-T7 antibody followed by western blot with anti-T7 antibody.

FIG. 11 shows that the HIV-1 IN C-terminal domain alone is sufficient for binding to imp7 and to inhibit HIV-1 infection.

11A) Schematic representation of CMV-YFP and CMV-YFP-INc205 (IN amino acids 205-288) expressors.

11B) intracellular localization of YFP or YFP-INc205 in HeLa cells.

11C) The HIV-1 IN C-terminal domain alone is sufficient for binding to imp7. Plasmids expressing YFP, IN-YFP, or YFP-INc205 were cotransfected with CMV-T7-imp7. After 48 hrs of transfection, imp7-binding was analyzed using anti-GFP immunoprecipitation and subsequently western blot with anti-T7 or anti-GFP antibodies.

11D) Over-expression of the HIV-1 IN C-terminal domain alone is sufficient for inhibiting infection of VSV-G-pseudotyped HIV-1 virus in 293T cells. To test the effect of YFP-INc205 on HIV-1 infection, each 293T cell line, including parental 293T cells, was infected with equal amounts of VSV-G pseudotyped pNLlucΔBgII virus (at 5 cpm of RT activity/cell). Since viruses contain a luciferase (luc) gene in place of the nef gene, viral infection can be monitored by using a sensitive luc assay which could efficiently detect viral gene expression After 48 hours of infection, equal amounts of cells (1×10⁶ cells) were lysed in 50 μl of luc lysis buffer and then, 10 μl of cell lysates was used for measurement of luc activity.

FIG. 12 shows that mutations in the C-terminal domain of IN inhibit HIV single-cycle replication and affect reverse transcription and nuclear import.

12A) 293T cells were transfected with a RT, IN and Env deleted HIV-1 provirus NLlucΔBglΔRI with different Vpr-RT-IN(WT/Mutant) expressors and a VSV-G expresser. Produced viruses (lane 1 to 3) were lysed and directly loaded in 12% SDS-PAGE and analyzed by Western blot with human anti-HIV serum. The positions of HIV-1 Gag, RT and IN proteins are indicated.

12B) The CD4+ C8166 cells were infected with viruses vWT, vD64E, and vKKRK viruses. At different time intervals after infection, the equal amount (1×10⁶) of cells was collected and cell-associated luciferase activity was measured by luciferase assay.

12C) Effects of Imp7-binding defect mutants on HIV-1 reverse transcription and DNA nuclear import. At 24 hours post-infection, 2×10⁶ cells were gently lysed and fractionated into the cytoplasmic and the nuclear fractions. The amount of viral DNA in both fractions were analyzed by PCR using HIV-1 LTR-Gag primers and Southern blot. Nuc. nuclear fraction; Cyt. cytoplasmic fraction, The purity and DNA content of each subcellular fraction were monitored by PCR detection of human globin DNA and visualized by specific Southern blot (lower panel).

12D) The total amounts of viral DNA (right panel) and the percentage of nucleus-associated viral DNA relative to the total amount of viral DNA (left panel) for each mutant was also quantified by laser densitometry. Means and standard deviations from two independent experiments are shown.

FIG. 13 shows siRNA-mediated silencing of Imp7 inhibits HIV-1 infection.

13A) A schematic depiction of the method steps shown as an example.

13B) siRNA-mediated silencing of Imp7 in 293T and HeLa-β-Gal-CD4/CCR5 cells. Cells were transfected with 20 nM of siRNA at 0 and 18 hours. After 48, 72 and 96 hours post initial transfection, the Imp7 expression levels in the cells were verified by Western blot with anti-Imp7 antibody (upper panel). Meanwhile, the expression of α-tubulin was also verified (lower panel).

13C) 293T cells were treated with sc-RNA or si-imp7 once a day for two days and used to produced VSV-G-pseudotyped HIV-1 4.3 virus (sc-virus and si-virus). Both viruses were then used to infect HeLa-β-Gal-CD4/CCR5 cells that have been treated with Imp7 siRNA or scramble RNA for 72 h. Luciferase activity was measured at 48 h post-infection.

13D) sc-RNA or si-imp7 treated HeLa-β-Gal cells were infected with wild-type enveloped HxBru virus produced from sc-RNA- or si-imp7-treated HeLa cells. Viral Infection was evaluated by MAGI assay.

FIG. 14 shows subcellular localization of the wild-type and truncated HIV integrase fused with YFP.

14A) Schematic structure of HIV-1 integrase-YFP fusion proteins. Full-length (1-288aa) HIV-1 integrase, the N-terminus-truncated mutant (51-228aa) or the C-terminus-truncated mutant (1-212aa) was fused in frame at the N-terminus of YFP protein. The cDNA encoding for each IN-YFP fusion protein was inserted in a SVCMV expression plasmid.

14B) Expression of different IN-YFP fusion proteins in 293T cells. 293T cells were transfected with each IN-YFP expressor and at 48 hours of transfection, cells were lysed, immunoprecipitated with anti-HIV serum and resolved by electrophoresis through a 12.5% SDS-PAGE followed by Western blot with rabbit anti-GFP antibody. The molecular weight markers are indicated at the left side of the gel.

14C) Intracellular localization of different IN-YFP fusion proteins. HeLa cells were transfected with each HIV-1 IN-YFP fusion protein expressor and at 48 hours of transfection, cells were fixed and subjected to indirect immuno-fluorescence using rabbit anti-GFP and then incubated with FITC-conjugated anti-rabbit antibodies. The localization of each fusion protein was viewed by Fluorescence microscopy with a 50× oil immersion objective. Upper panel is fluorescence images and bottom panel is DAPI nucleus staining.

FIG. 15 shows the effect of different IN C-terminal substitution mutants on IN-YFP intracellular localization.

15A) Diagram of HIV-1 IN domain structure and introduced mutations at the C-terminal domain of the protein. The position of lysines in two tri-lysine regions and introduced mutations are shown at the bottom of sequence. The IN sequence shown corresponds to amino acids 210-288 of SEQ ID NO:1.

15B) The expression of the wild-type and mutant IN-YFP fusion proteins were detected in transfected 293T cells by using immunoprecipitation with anti-HIV serum and Western blot with rabbit anti-GFP antibody, as described in FIG. 1. The molecular weight markers are indicated at the left side of the gel.

15C) Intracellular localization of different HIV-1 IN mutant-YFP fusion proteins in HeLa cells were analyzed by fluorescence microscopy with a 50× oil immersion objective. The nucleus of HeLa cells was simultaneously visualized by DAPI staining (lower panel).

FIG. 16 shows the production of different single-cycle replicating viruses and their infection in HeLa-CD4-CCR5-β-Gal cells.

16A) To evaluate the trans-incorporation of RT and IN in VSV-G pseudotyped viral particles, viruses released from 293T cells transfected with NLlucΔBglΔRI provirus alone (lane 6) or cotransfected with different Vpr-RT-IN expressors and a VSV-G expresser (lane 1 to 5) were lysed, immunoprecipitated with anti-HIV serum. Immunoprecipitates were run in 12% SDS-PAGE and analyzed by Western blot with rabbit anti-IN antibody (middle panel) or anti-RT and anti-p24 monoclonal antibody (upper and lower panel).

16B) The infectivity of trans-complemented viruses produced in 293 T cells was evaluated by MAGI assay. HeLa-CD4-CCR5-LTR-β-Gal cells were infected with equal amounts (at 10 cpm/cell) of different IN mutant viruses and after 48 hours of infection, numbers of β-Gal positive cells (infected cell) were monitored by X-gal staining. Error bars represent variation between duplicate samples and the data is representative of results obtained in three independent experiments.

FIG. 17 shows the effect of IN mutants on viral infection in dividing and nondividing C8166 T cells. To test the effect of different IN mutants on HIV-1 infection in CD4+ T cells, dividing (panel A) and non-dividing (aphidicolin-treated, panel B) C8166 T cells were infected with equal amount of VSV-G pseudotyped IN mutant viruses (at 5 cpm/cell). For evaluation of the effect of different IN mutants on HIV-1 envelope-mediated infection in CD4+ T cells, dividing C8166 T cells were infected with equal amount of HIV-1 envelope competent IN mutant viruses (at 10 cpm/cell) (panel C). After 48 hours of infection, HIV-1 DNA-mediated luciferase induction was monitored by luciferase assay. Briefly, the same amount (10⁶ cells) of cells was lysed in 50 ul of luciferase lysis buffer and then, 10 μl of cell lysate was subjected to the luciferase assay. Error bars represent variation between duplicate samples and the data is representative of results obtained in three independent experiments.

FIG. 18 shows the effects of different IN mutants on HIV-1 reverse transcription and DNA nuclear import.

Dividing C8166 T cells were infected with equal amounts of different HIV-1 IN mutant viruses.

18A) At 12 hours post-infection, 1×10⁶ cells were lysed and the total viral DNA was detected by PCR using HIV-1 LTR-Gag primers and Southern blot.

18B) Levels of HIV-1 late reverse transcription products detected in panel A were quantified by laser densitometry and viral DNA level of the wt virus was arbitrarily set as 100%. Means and standard deviations from two independent experiments are presented.

18C) At 24 hours post-infection, 2×10⁶ cells were fractionated into cytoplasmic and nuclear fractions as described in Materials and Methods. The amount of viral DNA in cytoplasmic and nuclear fractions were analyzed by PCR using HIV-1 LTR-Gag primers and Southern blot (upper panel, N. nuclear fraction; C. cytoplasmic fraction). Purity and DNA content of each subcellular fraction were monitored by PCR detection of human globin DNA and visualized by specific Southern blot (lower panel).

18D). The percentage of nucleus-associated viral DNA relative to the total amount of viral DNA for each mutant was also quantified by laser densitometry. Means and standard deviations from two independent experiments are shown.

FIG. 19 shows the effect of IN mutants on HIV-1 proviral DNA integration. Dividing C8166 T cells were infected with equal amounts of different HIV-1 IN mutant viruses. At 24 hours post-infection, 1×10⁶ cells were lysed and serial-diluted cell lysates were analyzed by two-step Alu-PCR and Southern blot for specific detection of integrated proviral DNA from infected cells (Upper panel). The DNA content of each lysis sample was also monitored by PCR detection of human β-globin DNA and visualized by specific Southern blot (middle panel). The serial-diluted ACH-2 cell lysates were analyzed for integrated viral DNA and as quantitative control (lower panel). The results are representative for two independent experiments.

FIG. 20 shows that expression of HIV-1 integrase C-terminal domain in viral producer cells inhibits subsequent HIV-1 infection in HeLa-β-Gal-CD4-CCR5 cells and in CD4⁺ T-lymphoid MT4 cells. 293T cells were transfected with HIV-1 provirus NL4.3-Nef+/GFP+ and SVCMVin-T7 or SVCMVin-T7-IN_{C205-288} expressor (the IN sequence shown as SEQ ID NO:2). After 48 hours of transfection, viruses were collected from the supernatant through an ultracentrifugation, and virus titers were quantified by HIV-1 RT activity assay. Equal amounts of viruses, as measured by virion-associated reverse transcriptase activity (A), were used to infect HeLa-β-Gal-CD4/CCR5 cells (B) or MT4 cells (C). At 48 h post-infection, the viral infection levels were evaluated by MAGI assay (B) or by counting of GFP-positive cells (C).

FIG. 21 shows the amino acid sequence of HIV-1 integrase (SEQ ID NO:1 derived from HIV-1 pNL4.3 strain) shown as an example. The C-terminal domain of IN and the two tri-lysine regions and an arginine/lysine region involved in IN/imp7 and IN/impβ interactions are indicated.

FIG. 22 shows fusions of Tat peptide (SEQ ID NO:9) with IN peptides (SEQ ID NOs 10-15) as examples.

FIG. 23 shows the siRNA target regions of IN as examples. The HIV-1 IN RNA sequence from nt 628 to 801 is shown (SEQ ID NO:16); this sequence encodes amino acids 210 to 267 of integrase. Also indicated are the RNA sequence encoding the two tri-lysine regions and the arginine/lysine rich region. These sequences (siRNA #1-4; SEQ ID NOs 17-20) can be used for siRNA silencing of IN protein expression during viral replication.

FIG. 24 is an ELISA scheme based on INc205-288 as an example, for screening of compounds that inhibit IN interaction with imp7 and/or with impβ.

FIG. 25 is a schematic depiction of a live cell BRET assay used as an example for detecting interaction of the C-terminal domain of HIV-1 IN with impβ and imp7 in live cells.

### DETAILED DESCRIPTION OF EMBODIMENTS

The invention has to do with using the C-terminal domain of HIV-1 integrase, and certain peptides derived from specific regions of this domain, for inhibiting HIV-1 infection. Without being limited by mechanism, the invention is based on the finding that these regions of the IN C-terminal domain interact directly with the nuclear import machinery of the host cell, specifically with imp7 and impβ, and that these IN regions are necessary for translocating the HIV-1 nucleoprotein pre-integration complex (PIC) into the nucleus. Thus these IN regions are important for establishing HIV replication and subsequent infection.

The experiments carried out herein make use of certain specific materials and techniques. These are set forth below solely for verifying the experimental findings and should not limit the scope of the invention.

(1) Construction of different IN expressors and HIV-1 RT/IN defective provirus: The full-length wild-type HIV-1 IN cDNA was amplified by polymerase chain reaction (PCR) using HIV-1 HxBru strain [Yao et al. J Virol. 1995; 69:7032-7044] as template and an engineered initiation codon (ATG) was placed prior to the first amino acid (aa) of IN. The primers are 5′-IN-HindIII-ATG (5′-GCGCAAGCTTGGATAGATGTTTTTAGATGGAA-3′; SEQ ID NO:23) and 3′-IN-Asp718 (5′-CCATGTGTGGTACCTCATCCTGCT-3′; SEQ ID NO:24). The PCR product was digested with HindIII and Asp718 restriction enzymes and cloned in frame to 5′ end of EYFP cDNA in a pEYFP-N1 vector (BD Biosciences Clontech) and generated a IN-YFP fusion expressor. Also, cDNA encoding for truncated IN (aa 50 to 288 or aa 1 to 212) was amplified by PCR and also cloned into pEYFP-N1 vector. The primers for generation of IN50-288 cDNA are IN50-HindIII-ATG-5′ (5′-GCGCAAGCTTGGATAGATGCATGGACAAGTAG-3; SEQ ID NO:25) and 3′-IN-Asp718 and primers for amplifying IN1-212 cDNA are IN-HindIII-ATG-5′ and IN-212-XmaI-3′ (5′-CAATTCCCGGGTTTGTATGTCTGTTTGC-3; SEQ ID NO:26). IN substitution mutants IN_{KK215,9AA}-YFP, IN_{KK240,4AE}-YFP and IN_{RK263,4AA}-YFP, were generated by a two-step PCR-based method [Yao et al. Gene Ther. 1999; 6:1590-1599] by using a 5′-primer (5′-IN-HindIII-ATG), a 3′-primer (3′-IN-Asp718) and complementary primers containing desired mutations. Amplified IN cDNAs harboring specific mutations were then cloned into pEYFP-N1 vector. To improve the expression of each IN-YFP fusion protein, all IN-YFP fusing cDNAs were finally subcloned into a SVCMV vector, which contains a cytomegalovirus (CMV) immediate early gene promoter [Yao et al. Gene Ther. 1999; 6:1590-1599].

To construct HIV-1 RT/IN defective provirus NLlucΔBglΔRI, we used a previously described HIV-1 envelope-deleted NLlucΔBglD64E provirus as the backbone. In this provirus, the nef gene was replaced by a firefly luciferase gene [Poon et al. J Virol. 2003; 77:3962-3972]. The ApaI/SalI cDNA fragment in NLlucBglD64E was replaced by the corresponding fragment derived from a HIV-1 RT/IN deleted provirus R-/ΔRI [Ao et al. J Virol. 2004; 78:3170-3177] and generated a RT/IN deleted provirus NLlucΔBglΔRI, in which RT and IN gene sequences were deleted while a 194-bp sequence harboring cPPT/CTS cis-acting elements was maintained. To restore HIV-1 envelope gene sequence in NLlucΔBglΔRI provirus, the SalI/BamHI cDNA fragment in this provirus was replaced by a corresponding cDNA fragment from a HIV-1 envelope competent provirus R-/ΔRI [Ao et al. J Virol. 2004; 78:3170-3177] and the resulting provirus is named as NLlucΔRI. To functionally complement RT/IN defects of NLlucΔBglΔRI, a CMV-Vpr-RT-IN fusion protein expressor [Ao et al. J Virol. 2004; 78:3170-3177] was used in this study. Co-transfection of NLlucΔBglΔRI, CMV-Vpr-RT-IN and a vesicular stomatitis virus G (VSV-G) glycoprotein expressor results in the production of VSV-G pseudotyped HIV-1 that can undergo for single cycle replication in different cell types [Ao et al. J Virol. 2004; 78:3170-3177]. To investigate the effect of IN mutants on viral replication, different mutants KK215,9AA, KK240.4AE, RK263,4AA or D64E were introduced into CMV-Vpr-RT-IN expressor by PCR-based method as described above and using a 5′-primer corresponding to a sequence in RT gene and including a natural NheI site (5′-GCAGCTAGCAGGGAGACTAA-3′; SEQ ID NO:27), a 3′-primer (3′-IN-stop-PstI, 5′-CTGTTCCTGCAGCTAATCCTCATCCTG-3′; SEQ ID NO:28) and the complementary oligonucleotide primers containing desired mutations. All IN mutants were subsequently analyzed by DNA sequencing to confirm the presence of mutations or deletions.

(2) Cell lines and reagents: Human embryonic kidney 293T, HeLa and HeLa-CD4-CCR5-β-Gal cells were maintained in Dulbecco's Modified Eagles Medium (DMEM) supplemented with 10% fetal calf serum (FCS). Human C8166 T-lymphoid cells were maintained in RPMI-1640 medium. Antibodies used in the immunofluorescent assay, immunoprecipitation or western blot are as follows: The HIV-1 positive human serum 162 and anti-HIVp24 monoclonal antibody used in this study were previously described [Yao et al. J Virol. 1998; 72:4686-4693]. The rabbit anti-GFP and anti-IN antibodies were respectively obtained from Molecular Probes Inc and through AIDS Research Reference Reagent Program, Division of AIDS, NIAID, NIH. Aphidicolin was obtained from Sigma Inc.

(3) Cell transfection and immunofluorescence assay: DNA transfection in 293T and HeLa cells were performed with standard calcium phosphate DNA precipitation method. For immunofluorescence analysis, HeLa cells were grown on glass coverslip (12 mm²) in 24-well plate. After 48 h of transfection, cells on the coverslip were fixed with PBS-4% paraformaldehyde for 5 minutes, permeabilized in PBS-0.2% Triton X-100 for 5 minutes and incubated with primary antibodies specific for GFP or HIV-1 IN followed by corresponding secondary FITC-conjugated antibodies. Then, cells on the coverslip were viewed using a computerized Axiovert 200 inverted fluorescence microscopy (Becton Deckson Inc).

(4) Virus production and infection: Production of different single-cycle replicating virus stocks and measurement of virus titer were previously described [Ao et al. J Virol. 2004; 78:3170-3177]. Briefly, 293T cells were co-transfected with RT/IN defective NLlucΔBglΔRI provirus, a VSV-G expressor and each of CMV-Vpr-RT-IN (wt/mutant) expressor.

To produce HIV-1 envelope competent single cycle replicating virus, 293T cells were co-transfected with NLlucRI and different CMV-Vpr-RT-IN (wt/mutant) expressors. After 48 hours of transfection, supernatants were collected and virus titers were quantified by RT activity assay [Yao et al. Gene Ther. 1999; 6:1590-1599].

To test the effect of IN mutants on virus infection, equal amounts of virus were used to infect HeLa-CCR5-CD4-β-Gal cells, dividing and non-dividing C8166 T cells. To compare the infection of each viral stock in HeLa-CCR5-CD4-β-Gal cells, numbers of infected cells (β-Gal positive cells) were evaluated by the MAGI assay 48 hours post-infection (p.i) as described previously [Kimpton et al. J Virol. 1992; 66:2232-2239]. To infect CD4+ T cells, dividing or aphidicolin-treated non-dividing C8166 T cells (with 1.3 μg/ml of aphidicolin) were infected with equivalent amounts of single cycle replicating viruses (5 cpm/cell) for 2 hours. Then, infected cells were washed and cultured in the absence or presence of the same concentration of aphidicolin. At 48 hours post-infection, 1×10⁶ cells from each sample were collected, washed twice with PBS, lysed with 50 μl of luciferase lysis buffer (Fisher Scientific Inc) and then, 10 μl of cell lysate was subjected to the luciferase assay by using a TopCount®NXT™ Microplate Scintillation & Luminescence Counter (Packard, Meriden) and the luciferase activity was valued as relative luciferase units (RLU). Each sample was analyzed in duplicate and the average deviation was calculated.

(5) Immunoprecipitation and Western blot analyses: For detection of IN-YFP fusion proteins, 293T cells transfected with each IN-YFP expressor were lysed with RIPA lysis buffer and immunoprecipitated using human anti-HIV serum. Then, immunoprecipitates were run in 12% SDS-PAGE and analyzed by Western blot using rabbit anti-GFP antibody. To analyze virion-incorporation of IN and virus composition, 293T cells were co-transfected with NLlucΔBglΔRI provirus and each of CMV-Vpr-RT-IN (wt/mutant) expressors. After 48 hours, viruses were collected, lysed with RIPA lysis buffer and immunoprecipitated with human anti-HIV serum. Then, immunoprecipitates were run in 12% SDS-PAGE and analyzed by Western blot with rabbit anti-IN antibody and anti-p24 monoclonal antibody or anti-HIV serum.

(6) HIV-1 reverse-transcribed and integrated DNA detection by PCR and Southern blotting: C8166 T cells were infected with equal amount of the wt or IN mutant viruses for 2 hours, washed for three times and cultured in RPMI medium. To detect total viral DNA synthesis, at 12 hours post-infection, equal number (1×10⁶ cells) of cells were collected, washed twice with PCR washing buffer (20 mM Tris-HCl, pH 8.0, 100 mM KCl), and lysed in lysis buffer (PCR washing buffer containing 0.05% NP-40, 0.05% Tween-20).

Lysates were then incubated at 56° C. for 30 min with proteinase K (100 μg/ml) and at 90° C. for 10 min prior to phenol-chloroform DNA purification. To detect viral cDNA from each sample, all lysates were serially diluted 5-fold and subjected to PCR analysis. The primers used to detect late reverse transcription products were as follows: 5′-LTR-U3,5′-GGATGGTGCTTCAAGCTAGTACC-3′ (SEQ ID NO:29; nt position 8807, +1=start of BRU of transcription initiation); 3′-Gag 5′-ACTGACGCTCTCGCACCCATCTCTCTC-3′ (SEQ ID NO:30; nt position 329). The probe for southern blot detection was generated by PCR with a 5′-LTR-U5 oligonucleotide, 5′-CTCTAGCAGTGGCGCCCGAACAGGGAC-3′ (SEQ ID NO:31; nt position 173) and the 3′-Gag oligo. PCR was carried out using 1× HotStar Taq Master Mix kit (QIAGEN, Mississauga, Ontario), as described previously [Ao et al. J Virol. 2004; 78:3170-3177].

To analyze nucleus- and cytoplasm-associated viral DNA, a subcellular fractionation of infected C8166 T cells (2×10⁶) was performed after 24 hours of infection, as described previously [Simon et al. J Virol. 1996; 70:5297-5305]. Briefly, infected cells were pelleted and resuspended in ice-cold PCR lysis buffer (washing buffer containing 0.1% NP-40). After a 5-min incubation on ice, the nucleus was pelleted by centrifugation, washed twice with PCR wash buffer, and lysed in lysis buffer (0.05% NP-40, 0.05% Tween-20). Then, both cytoplasmic sample (supernatant from the first centrifugation) and the nuclear sample were treated with proteinase K and used for PCR analysis, as described above.

Integrated proviral DNA was detected in cell lysates by a modified nested Alu-PCR [Ao et al. J Virol. 2004; 78:3170-3177], in which following the first PCR, a second PCR was carried-out to amplify a portion of the HIV-1 LTR sequence from the first Alu-LTR PCR-amplified products. The first PCR was carried out by using primers including 5′-Alu oligo (5′-TCCCAGCTACTCGGGAGGCTGAGG-3′; SEQ ID NO:32) and 3′-LTR oligo (5′-AGGCAAGCTTTATTGAGGGCTTAAGC-3′; SEQ ID NO:33) (nt position 9194) located respectively in the conserved region of human Alu sequence and in HIV-1 LTR. The primer used for both of the second nested PCR and for generating a probe are 5′-NI: 5′-CACACACAAGGCTACTTCCCT-3′ (SEQ ID NO:34) and 3′-NI: 5′-GCCACTCCCCAGTCCCGCCC-3′ (SEQ ID NO:35). As a control, the first and second PCR primer pairs were also used in parallel to detect integrated viral DNA from serially diluted ACH-2 cells, which contain one viral copy/cell, in a background of uninfected C8166 cellular DNA.

To evaluate the DNA content of extracted chromosomal DNA preparations, detection of human β-globin gene was carried-out by PCR, as described previously [Simon et al. J Virol. 1996; 70:5297-5305]. All final PCR products were electrophoresed through 1.2% agarose gel and transferred to hybridization transfer membrane (GeneScreen Plus, PerkinElmer Life Sciences), subjected to Southern hybridization by using specific PCR DIG-Labeling probes (Roche Diagnostics, Laval, Que) and visualized by a chemiluminescent method. Densitometric analysis was performed using a Personal Molecular Imager (Bio-Rad) and Quantity One software version 4.1.

(7) Interaction of HIV-1 IN and importin 7. We investigated the interaction of HIV-1 IN with different cellular nuclear import factors. We first tested the interaction of HIV-1 IN with cellular nuclear import receptors Imp7 and Imp8, by using a cell-based-co-immunoprecipitation (co-immunoprecipitation) assay. SVCMV-T7-Imp7 and T7-Imp8 expressors were constructed by inserting Imp7 and Imp8 cDNAs into a SVCMV-T7 vector at the 3′ end of a T7 tag encoding sequence (FIG. 1A). The HIV-1 IN-YFP fusion protein, expressed from expresser CMV-IN-YFP, and YFP expressed from the CMV-YFP expressor, were used in the study and shown in FIG. 1A. First, expression of these proteins was checked by transfecting each of these expressors into 293T cells, and processed using anti-GFP or anti-T7 immunoprecipitation (IP), followed by western blot with corresponding antibodies. Results showed that IN-YFP and YFP were detected at positions 58 and 27 kDa respectively (FIG. 1B, lanes 2 and 3), while T7-Imp7 and T7-Imp8 were at positions that ranged between 110 to 130 kDa (FIG. 1B, lanes 4 and 5). To test whether IN-YFP could bind to different importins, the YFP or IN-YFP expressor was co-transfected with each importin expresser in 293T cells, as indicated in FIG. 1C.

After 48 h, cells were lysed with CHAPS lysis buffer (199 medium containing 0.5% CHAPS), and immunoprecipitated using rabbit anti-GFP antibody. Precipitated complexes were run on an SDS-PAGE, followed by western blot with anti-T7 antibody (FIG. 1C, upper panel). Results revealed that, while YFP protein did not co-precipitate with any importin (FIG. 1C, upper panel, lane 1, 2), the immunoprecipitation of IN-YFP specifically co-pulled down T7-Imp7 (FIG. 1C, Lane 3), but not T7-Imp8 (FIG. 1C, lanes 4). Meanwhile, the immunoprecipitated IN-YFP and YFP in each sample respectively were checked by anti-GFP western blot, and similar levels of each protein were detected (FIG. 1C; middle panel, lanes 3, 4). To rule out the possibility that the co-precipitated T7-Imp7 was due to differential levels of importin expression in each transfection sample, the cell lysates were processed using sequential immunoprecipitation with anti-T7 antibody followed by anti-T7 Western blot, and the results showed similar expression levels of each importin in different samples (FIG. 1C; lower panel). All of these results indicated that IN specifically interacts with Imp7, but not with Imp8.

(8) HIV-1 IN interacts with endogenous imp7 and the interaction between IN and impβ takes place in the cells. We asked was whether the IN/Imp7 interaction occurs in the cells or after cells had been lysed. To address this question, IN-YFP or T7-Imp7 expressor was individually transfected into different 293T cell cultures, as indicated in FIG. 2A. After 48 hours, cells from two transfected cultures were mixed, lysed with 0.5% CHAPS lysis buffer and incubated in 4° C. for two hours. Then, the presence of IN/Imp7 interaction in the cell lysate was checked by anti-GFP immunoprecipitation, followed by anti-T7 western blot. In parallel, cells co-transfected with both IN-YFP and T7-Imp7 expressors were mixed with the same amounts of mock-transfected cells and processed identically. Strikingly, the co-precipitated T7-Imp7 was only detected in co-transfected cell lysate, but not in mixed cell lysate from individually transfected cell samples (FIG. 2A, upper panel, compare lane 2 with 3). These results clearly indicate that the interaction of IN-YFP and T7-Imp7 takes place in the cells.

Again, the specific detection of IN/Imp7 complex in co-transfected cells, was not due to the varying levels of expression of IN-YFP or T7-Imp7 protein in the different samples (FIG. 2 A, middle panel and lower panel; lanes 2 and 3). To further test the interaction between IN-YFP and endogenous Imp7, 293T cells were transfected with CMV-YFP or CMV-IN-YFP expressor, lysed by 0.5% CHAPS lysis buffer and immunoprecipitated with anti-GFP. The co-precipitated endogenous Imp7 was checked by western blot with a rabbit anti-human Imp7 antibody. Meanwhile, the non-transfected 293T cell lysates were directly loaded into SDS-PAGE as the positive control (FIG. 2B, lane 1). We found that IN-YFP, but not YFP, was able to pull down the endogenous Imp7 (FIG. 2B, upper panel, compare lane 4 to lane 3), indicating that IN-YFP interacts with endogenous Imp7 in 293T cells.

(9) In vitro interaction between IN and imp7. We asked whether IN binding to Imp7 could be through direct protein interaction. We produced purified recombinant GST and GST-Imp7 proteins in an E coli expression system, and the purified protein in each sample was tested by directly loading protein samples in an SDS-PAGE, and verified by Coomassie Blue staining of the gel (FIG. 3A) and by western blot with specific anti-Imp7 antibody. To test the direct interaction of IN and Imp7 in vitro, similar amounts of purified GST and GST-Imp7 were incubated with a purified recombinant HIV-1 IN in 199 medium containing 0.1% CHAPS for 2 h at 4° C., followed by an additional one hour incubation with glutathione-sepharose 4B beads. Then, the bound protein complex was eluted out with 10 mM glutathione, and loaded onto a 12.5% SDS-PAGE gel, followed by western blot analysis with anti-IN antibodies. Results showed that the purified HIV-1 IN, in both of dimer and monomer forms, was able to specifically interact with GST-Imp7, and not with GST (FIG. 3Bl). Thus, the binding of IN to Imp7 may be through a direct protein/protein interaction.

(10) Differential binding ability of HIV-1 MAp17 and IN to cellular importins Rch1 and imp7. The importin α/β nuclear translocation pathway has been implicated in assisting with HIV-1 nuclear import. Several HIV-1 proteins, including MAp17, Vpr and IN have been shown to be able to interact with Impα in in vitro binding assays. In this study, we attempted to test whether HIV-1 IN could interact with Rch1, a member of the human importin a family, by using co-immunoprecipitation assay. A T7-tagged Rch1 expressing plasmid (CMV-T7-Rch1), and an HIV-1 MAp17_{G2A} mutant-YFP fusion protein expressing plasmid (CMV-MA_{G2A}-YFP) were constructed. In MAp17_{G2A}-YFP, the second amino acid glycine in MAp17 protein was replaced by alanine, and this MAp17 mutant was previously shown to capable of binding to Rch1 in a cell-based co-immunoprecipitation system. After IN-YFP or MA_{G2A}-YFP were co-expressed with T7-Rch1 in 293T cells, their interaction with Rch1 was analyzed using the same co-immunoprecipitation and western blot protocols, as described in FIG. 1. MA_{G2A}-YFP was shown to be able to bind to T7-Rch1 (FIG. 4A; lane 4). However, IN-YFP did not show any interaction with T7-Rch1 (FIG. 4A, lane 3). In contrast, while T7-Imp7 co-precipitated with IN-YFP, no T7-Imp7 was detected in the immunoprecipitated MA_{G2A}-YFP sample (FIG. 4B, compare lane 4 to 3). These results suggest that HIV-1 IN and MAp17 may interact with different cellular nuclear import factors during HIV-1 replication.

(11) Delineation of necessary region(s) of HIV-1 IN for its interaction with imp7. To delineate which region(s) within HIV-1 IN is required for its Imp7-binding, we first tested an IN N-terminal deletion mutant expressed from the CMV-IN₅₀₋₂₈₈-YFP expressor (FIG. 5A) for Imp7-binding. Co-immunoprecipitation analysis revealed that, similar to the IN-YFP, IN₅₀₋₂₈₈-YFP also bound efficiently to T7-Imp7 (FIG. 5B, compare lane 5 to lane 4), indicating that the N-terminal domain of IN is not required for IN/Imp7 interaction.

To test the core domain and the C-terminal domain of IN for their contribution towards Imp7-binding, we constructed three YFP-IN expressors, including CMV-YFP-INwt and two IN C-terminal deletion mutants (CMV-YFP-IN1-212 and CMV-YFP-IN1-240) (FIG. 5A). With the CMV-YFP-INwt expressor, the PCR-amplified HIV-1 IN full length cDNA, was placed in frame at the 3′ end of the YFP-cDNA, while for CMV-YFP-IN1-212 and CMV-YFP-IN1-240, sequences encoding for the last 76 and 48 aa of IN was removed respectively. Expression of each YFP-IN fusion protein along with its ability to bind Imp7 was tested in 293T cells by co-transfecting each YFP-IN fusion protein expressor with the T7-Imp7 plasmid. The YFP-INwt, YFP-IN1-212 and CMV-YFP-IN1-240 fusion proteins were detected at molecular weights ranging approximately from 47 to 58 kDa (FIG. 5C, middle panel, lanes 3 to 5). Co-immunoprecipitation experiments revealed that while YFP-INwt efficiently bound to T7-Imp7, the two IN C-terminal deletion mutants were unable to bind to T7-Imp7 (FIG. 5C, upper panel, compare lane 3 to lanes 4 and 5), suggesting that the C-terminal region encompassing residues 240 and 288 is required for IN interacting with Imp7.

(12) Subcellular localization of the wild-type and truncated HIV integrase fused with YFP. We investigated the intracellular localization of HIV-1 IN and delineated the region(s) of IN contributing to its karyophilic property. A HIV-1 IN-YFP fusion protein expressor (CMV-IN-YFP) was generated by fusing a full-length HIV-1 IN cDNA (amplified from HIV-1 HxBru molecular clone, see Yao et al. J Virol. 1995; 69:7032-7044) to the 5′ end of YFP cDNA in a CMV-IN-YFP expresser. Transfection of CMV-IN-YFP expresser in 293T cells resulted in the expression of a 57 kDa IN-YFP fusion protein (FIG. 14B, lane 2; FIG. 15B, lane 1), whereas expression of YFP alone resulted in a 27 kDa protein (FIG. 15B, lane 5). Given that HeLa cells have well-defined morphology and are suitable for observation of intracellular protein distribution, we tested the intracellular localization of YFP and IN-YFP by transfecting CMV-IN-YFP or CMV-YFP expresser in HeLa cells. After 48 hours of transfection, cells were fixed and subjected to indirect immunofluorescence assay using primary rabbit anti-GFP antibody followed by secondary FITC-conjugated anti-rabbit antibodies. Results showed that, in contrast to a diffused intracellular localization pattern of YFP (data not shown), the IN-YFP fusion protein was predominantly localized in the nucleus (FIG. 14C, a1), confirming the karyophilic feature of HIV-1 IN.

We constructed two truncated IN-YFP expressors, CMV-IN50-288-YFP and CMV-IN1-212-YFP. In CMV-IN50-288-YFP, the N-terminal HH-CC domain of IN (aa 1-49) was deleted and in CMV-IN1-212-YFP, the C-terminal domain (aa 213-288) was removed (FIG. 14A). Transfection of each truncated IN-YFP fusion protein expresser in 293T cells resulted in the expression of IN50-288-YFP and IN1-212-YFP at approximately 52 kDa and 48 kDa molecular mass respectively (FIG. 14B, lanes 3 and 4). We next investigated the intracellular localization of truncated IN-YFP fusion proteins in HeLa cells by using indirect immunofluorescence assay, as described above. Results showed that the IN50-288-YFP was predominantly localized in the nucleus with a similar pattern as the wild-type IN-YFP fusion protein (FIG. 14C, compare b1 to a1). However, IN1-212-YFP fusion protein was excluded from the nucleus, with an accumulation of the mutant protein in the cytoplasm (FIG. 14C, c1). These results were also further confirmed by using rabbit anti-IN antibody immunofluorescence assay. Taken together, our data show that the C-terminal domain of HIV-1 IN is required for its nuclear accumulation.

(13) Effect of different IN C-terminal substitution mutants on IN:imp7 interaction. We constructed several IN mutants in the IN C-terminal region, in the form of YFP-IN fusion proteins (FIG. 6A). Mutants YFP-IN_{240,4AA}, YFP-IN_{263,4AA} and YFP-IN_{KKRK} were designed to target a tri-lysine region (²³⁵WKGPA²⁴⁰KLLW²⁴⁴KG), and/or an arginine/lysine rich region (²⁶²RRKAK). The YFP-IN_{249, 50AA} and YFP-IN_{258A} mutants were constructed to target highly conserved residues valine and lysine at positions 249, 250 and 258 (FIG. 6A). An IN core domain mutant YFP-INKR186,7AA was also included in this study. Each YFP-IN mutant plasmid was co-transfected with the T7-Imp7 expressor in 293T cells, and processed by the co-immunoprecipitation assay to test each protein's Imp7-binding ability. Results revealed that while other IN mutants did not affect the ability to bind Imp7 (FIG. 6B, lanes 4, 5, 10), the YFP-IN263,4AA mutant significantly impaired the ability of IN to bind Imp7, and the YFP-INKKRK mutant was unable to interact with imp7 (FIG. 6B, lanes 9 and 10). Thus, all these results indicate that both tri-lysine region (²³⁵WKGPA²⁴⁰KLLW²⁴⁴KG) and the arginine/lysine rich region (²⁶²RRKAK) is required for efficient interaction between IN and Imp7.

(14) Effect of different IN C-terminal substitution mutants on IN-YFP intracellular localization. The C-terminal domain of HIV-1 IN contains several regions that are highly conserved in different HIV-1 strains, including Q, C and N regions. Regions C (²³⁵WKGPAKLLWKGEGAVV; SEQ ID NO:5) and N (²⁵⁹VVPRRKAK; SEQ ID NO:6) are conserved in all known retroviruses and the ²¹¹KELQKQITK (SEQ ID NO:3) motif falls within the so-called glutamine-rich based region (sequence Q) of lentiviruses. We term the sequences ²¹¹KELQKQITK (SEQ ID NO:3) and ²³⁶KGPAKLLWK (SEQ ID NO:4) the proximal tri-lysine region and distal tri-lysine region, respectively (FIG. 15A). The lysine residues in these regions are highly conserved in most HIV-1 strains [Kuiken et al. HIV Sequence Compendium 2001. Los Alamos National Laboratory. 2001]. To test whether these basic lysine residues could constitute for a possible nuclear localization signal for IN nuclear localization, we specifically introduced substitution mutations for two lysines in each tri-lysine region and generated IN_{KK215,9AA}-YFP and IN_{KK240,4AE}-YFP expressors (FIG. 15A). In the conserved N region, there is a stretch of four basic residues among five amino acids (aa) ²⁶²RRKAK. To characterize whether this basic aa region may contributes to IN nuclear localization, we replaced an arginine and a lysine at positions of 263 and 264 by alanines in this region and generated a mutant (IN_{RK263,4AA}-YFP). The protein expression of different IN-YFP mutants in 293T cells showed that, like the wild type IN-YFP, each IN-YFP mutant fusion protein was detected at similar molecular mass (57 kDa) in SDS-PAGE (FIG. 15B, lanes 1 to 4), while YFP alone was detected at position of 27 kDa (lane 5). Then, the intracellular localization of each IN mutant was investigated in HeLa cells by using similar methods, as described above. Results showed that, while the wild type IN-YFP and IN_{RK263,4AA}-YFP still predominantly localized to the nucleus (FIG. 15C, a1 and d1), both IN_{KK215,9AA}-YFP and IN_{KK240,4AE}-YFP fusion proteins were shown to distribute throughout the cytoplasm and nucleus, but with much less intensity in the nucleus (FIG. 15C, a1 and b1). These data suggest that these lysine residues in each tri-lysine regions are required for efficient HIV-1 IN nuclear localization.

(15) Interaction of HIV-1 IN with T7-impβ in co-transfected 293T cells. We investigated whether HIV-1 IN could also interact with impβ. We constructed the SVCMVin-T7-Impβ expressor by cloning PCR amplified impβ from pET30a-impβ.

Then, the SVCMVin-T7-Impβ was co-transfected with SVCMVin-YFP, SVCMVin-IN-YFP or SVCMVin-YFP-IN expressor in 293T cells. After 48 hours, cells were lysed with 199 medium containing 0.25% NP-40 and a protease inhibitor cocktail (Roche) on ice for 30 min and clarified by centrifugation at 13,000 rpm for 30 min at 4° C. Then, the supernatant was subjected to immunoprecipitation (IP) with rabbit anti-GFP antibody and immunoprecipitates were resolved by 10% SDS-PAGE gel followed by western blot using mouse anti-T7 or mouse anti-GFP antibodies, respectively. Also, the total T7-Impβ expression in cell lysates was sequentially immunoprecipitated with mouse anti-T7 antibody followed by western blot using the same antibody. Results showed that immunoprecipitation of IN-YFP and YFP-IN pulled down T7-Impβ (FIG. 7, lanes 2 and 3), whereas no T7-Impβ was detected in YFP-transfected or mock-transfected samples (lane 1 and 4). Again, the specific detection of IN/Impβ complex in co-transfected cells, was not due to the varying levels of expression of T7-Impβ protein in the different samples (FIG. 7, lower panel). These results indicate that IN-YFP and YFP-IN, but not YFP, are capable of binding to T7-Impβ.

(16) Immunocomplex of IN-YFP and endogenous impβ and imp7 in 293T cells. To rule out the possibility that the IN/Impβ interaction could be an artifact of overexpression of these proteins in cells, we tested whether HIV-1 IN (in IN-YFP and YFP-IN) could interact with endogenous Impβ. SVCMVin-IN-YFP or SVCMVin-YFP-IN expressor was transfected alone in 293T cells and after 48 hours of transfection, cells were lysed by 0.25% NP-40 lysis buffer and immunoprecipitated with anti-GFP followed by western blot with a rabbit anti-human Impβ antibody (Cat# SC-11367, Santa Cruz Biotechnology Inc).

The co-immunoprecipitation and western blot results revealed that the endogenous Impβ were co-precipitated with IN-YFP and YFP-IN (FIG. 8, middle panel, lanes 3 and 4), but not with YFP alone (lane 2). Meanwhile, the similar amount of YFP, IN-YFP and YFP-IN were detected through anti-GFP western blot. These results demonstrated that HIV-1 IN is capable of binding endogenous Impβ.

We investigated whether endogenous Imp7 could be present in co-precipitated IN/Impβ complex. We stripped the membrane (middle panel FIG. 8) and re-processed the western blot with anti-human Imp7 antibody. Results revealed that endogenous Imp7 could also be detected in IN-YFP and YFP-IN samples (FIG. 8, upper panel, lanes 3 to 4), but not in mock-transfected and YFP expressing sample (lanes 1 and 2). This indicates that endogenous Impβ and Imp7 could be detected in the same IN-precipitation samples.

(17) Interaction of HIV-1 IN with impβ in vitro. To further study the interaction of HIV-1 IN with Impβ, we constructed pET21-chloramphenicol acetyltransferase (pET21-T7-CAT), pET21-T7-IN and pET21-T7-Ran and produced these proteins using the TnT T7 coupled reticulocyte lysate system (Cat# L4610, Promega) and labelled them with [³⁵S]methionine (PerkinElmer). Then, the produced protein samples were analyzed with SDS-PAGE and each protein was shown at the corresponding molecular weight (shown in FIG. 9A, left panel). Also, we produced and purified GST, GST-Impα (Rch1), GST-imp7 and GST-Impβ from E. coli BL21. Protein expression was induced by adding isopropyl-1-β-D-thiogalactopyranoside (1 mM) for 3 h at 37° C. Bacteria were harvested, suspended in 35 ml of ice-cold column buffer, and broken by sonication (five 30-s pulses at 100 watts, Sonics & Materials, Inc.). The resulting lysates were centrifuged for 30 min at 13000 rpm and pass through a glutathione-sepharose 4B column (Amersham Pharmacia Biotech Inc). After being washing by column buffer, the bound GST and GST-Imp7 proteins were eluted by glutathione buffer (100 mM reduced glutathione (Roche), 120 mM NaCl, 100 mM Tris-HCl pH 8.5). Finally, the eluted protein was dialyzed in PBS to remove high concentration of glutathione. Each purified protein stock was verified by directly loading on a 12.5% SDS-PAGE followed by the Coomassie Blue staining (FIG. 9A, right panel; 9C, left panel).

To test the IN-binding ability of GST, GST-Impα (Rch1), GST-imp7 and GST-Impβ, equal amounts of recombinant GST, GST-Impα (Rch1), GST-imp7 and GST-Impβ were incubated with in vitro-translated [³⁵S]methionine-labeled T7-CAT, T7-IN and T7-Ran proteins (indicated in FIG. 9B) or a purified HIV-1 recombinant IN protein (Cat No. 9420, obtained through AIDS Research Reference Reagent Program, Division of AIDS, NIAID, NIH) in 199 medium containing 0.25% NP40, for 2 hours at 4° C. Then, 100 μl of glutathione-sepharose 4B beads were added and incubated for additional one hour. The beads were washed and the bound proteins were eluted with 50 mM glutathione, loaded onto a 12.5% SDS-PAGE and subsequently analyzed by autoradiography (FIG. 9B) or followed by western blot analysis with rabbit anti-IN antibodies (FIG. 9C, right panel).

The in vitro binding results revealed that GST and GST-Impα did not show any binding to T7-CAT, T7-IN and T7-Ran. There was binding of GST-impβ to T7-IN and T7-Ran, but not T7-CAT (FIG. 9B). Moreover, results in FIG. 9C showed that both GST-imp7 and Imps, but not GST alone, could pull down recombinant IN (FIG. 9C, right panel), suggesting a direct interaction between both GST-imp7 and Imps and HIV IN.

(18) The C-terminal domain of HIV-1 IN interacts with impβ in co-transfected 293T cells. We made different IN C-terminal deletion mutants to test whether the C-terminal domain of IN is required for IN interaction with impβ and which region(s) are necessary for their binding. In CMV-YFP-INwt expressor, the PCR-amplified HIV-1 IN full length cDNA was placed in frame at the 3′ of YFP cDNA, while in CMV-YFP-IN₁₋₂₁₂, CMV-YFP-IN1-240 and CMV-YFP-IN₁₋₂₆₂, sequences encoding for last 76, 48 or 26 aa of IN was removed respectively. In YFP-IN₅₀₋₂₈₈, the N-terminal domain (1 to 49 aa) of IN was deleted. To test the expression of each of YFP-IN fusion proteins along with their abilities to bind impβ, each YFP-IN fusion protein expresser was co-transfected with a T7-impβ expresser in 293T cells. After 48 hours of transfection, cells were lysed with 0.5% NP-40-199 medium and processed using the co-IP assay, as described above. Results (see FIG. 10) show that, like the wt YFP-IN, the YFP-IN₁₋₂₄₀, YFP-IN₁₋₂₆₂ and YFP-IN₅₀₋₂₈₈ efficiently bound to T7-impβ. However, CMV-YFP-IN₁₋₂₁₂ lost drastically its binding ability to T7-impβ, suggesting a region between 213-239 aa in the C-terminal domain of IN may be important for this viral/cellular protein interaction.

Our results provide evidence that 1) HIV-1 IN, in both YFP-IN and IN-YFP forms, is able to interact with endogenous impβ and imp7; 2) in vitro binding results suggest that this IN-impβ interaction may be through a direct protein-protein interaction; 3) the C-terminal domain of IN encompassing aa 213-239 may be necessary for its interaction with impβ.

(19) The HIV-1 IN C-terminal domain alone is sufficient for binding to imp7 and to inhibit HIV-1 infection. We determined that the HIV-1 IN C-terminal domain alone is sufficient for binding to imp7. We co-transfected YFP, IN-YFP, or YFP-INc205 expressors with CMV-T7-imp7. After 48 hrs of transfection, imp7-binding was analyzed using anti-GFP immunoprecipitation and subsequently western blot with anti-T7 or anti-GFP antibodies (FIG. 11C).

We also determined that over-expression of the HIV-1 IN C-terminal domain alone is sufficient for inhibiting infection of VSV-G-pseudotyped HIV-1 virus in 293T cells. We tested the effect of YFP-INc205 (containing the C-terminal IN residues 205-288) on HIV-1 infection by infecting each 293T cell line, including the YFP-INc205 expresser cells and the parental 293T cells, with equal amounts of VSV-G pseudotyped pNLlucΔBgII virus (at 5 cpm of RT activity/cell). Since viruses contain a luciferase (luc) gene in place of the nef gene, viral infection can be monitored by using a sensitive luc assay which could efficiently detect viral gene expression. After 48 hours of infection, equal amounts of cells (1×10⁶ cells) were lysed in 50 μl of luc lysis buffer and then, 10 μl of cell lysates was used for measurement of luc activity. Results (see FIG. 11D) showed that virus infection in 293T-YFP cells induced 110×10³ RLU of luc activity, which was 33% lower than that in parental 293T cells. In 293T-YFP-INc205 cells, HIV-1 infection only induced 24×10³ RLU of luc activity, that was only approximately 15% and 21% of levels of that in parental 293T and 293T-YFP cells. These observations indicate that expression of the C-terminal region of IN in 293T cells inhibited HIV-1 infection.

(20) Production of different single-cycle replicating viruses and their infection in HeLa-CD4-CCR5-β-Gal cells. To specifically analyze the effect of IN mutants in early steps of viral infection, we modified a previously described HIV-1 single-cycle replication system [Ao et al. J Virol. 2004; 78:3170-3177] and constructed a RT/IN/Env gene-deleted HIV-1 provirus NLlucΔBglΔRI, in which the nef gene was replaced by a firefly luciferase gene [Poon et al. J Virol. 2003; 77:3962-3972]. Co-expression of NLlucΔBglΔRI provirus with Vpr-RT-IN expressor and a vesicular stomatitis virus G (VSV-G) glycoprotein expressor will produce viral particles that can undergo a single-round of replication, since RT, IN and Env defects of provirus will be complemented in trans by VSV-G glycoprotein and Vpr-mediated RT and IN trans-incorporation [Ao et al. J Virol. 2004; 78:3170-3177]. This single cycle replication system allows us to introduce different mutations into IN gene sequence without differentially affecting viral morphogenesis and the activity of the central DNA Flap. After different IN mutations KK215,9AA, KK240,4AE and RR263,4AA were introduced into Vpr-RT-IN expressor, we produced VSV-G pseudotyped HIV-1 IN mutant virus stocks in 293T cells. In order to specifically investigate the effect of IN mutants on early steps during HIV-1 infection prior to integration, an IN class I mutant D64E was also included as control. After each viral stock was produced (as indicated in FIG. 16A), similar amounts of each virus stock (quantified by virion-associated RT activity) were lysed and virus composition and trans-incorporation of RT and IN of each virus stock were analyzed by Western blot analysis with anti-IN and anti-HIV antibodies. Results showed that all VSV-G pseudotyped IN mutant viruses had similar levels of Gagp24, IN and RT, as compared to the wild-type virus (FIG. 16A), indicating that trans-incorporation of RT and IN as well as HIV-1 Gag processing were not differentially affected by the introduced IN mutations.

To test the infectivity of different IN mutant viruses in HeLa-CD4-CCR5-LTR-β-Gal cells, we first compared the infectivity of VSV-G pseudotyped wild type virus and the D64E mutant virus. At 48 hours post-infection with equivalent amount of each virus stock (at 1 cpm RT activity/cell), the number of β-Gal positive cells was evaluated by MAGI assay, as described in Kimpton et al. J Virol. 1992; 66:2232-2239. Results showed that the number of infected cells (β-Gal positive cells) for D64E mutant reached approximately 14% of the wild type level. This result is consistent with a previous report showing that, in HeLa MAGI assay, the infectivity level of class I IN integration-defect mutant was approximately 20 to 22% of wild type level. It indicates that, even though the IN mutant D64E virus is defective for integrating viral DNA into host genome, tat expression from nucleus-associated and unintegrated viral DNAs can activate HIV-1 LTR-driven β-Gal expression in HeLa-CD4-CCR5-LTR-β-Gal cells. Indeed, several studies have already shown that HIV infection leads to selective transcription of tat and nef genes before integration. Therefore, this HeLa-CD4-CCR5-LTR-β-Gal cell infection system provides an ideal method for us to evaluate the effect of different IN mutants on early steps of viral infection prior to integration. We next infected HeLa-CD4-CCR5-LTR-β-Gal cells with different VSV-G pseudotyped IN mutant viruses at higher infection dose of 10 cpm RT activity/cell and numbers of β-Gal positive cells were evaluated by MAGI assay after 48 hours of infection. Interestingly, results showed that the IN mutant D64E virus infection induced the highest level of β-Gal positive cells, whereas infection with viruses containing IN mutants KK215,9AA, KK240,4AE or RK263,4AA yielded much lower levels of β-Gal positive cells, which only reached approximately 11%, 5% or 26% of the level of D64E virus infection (FIG. 16B). Based on these results, we reasoned that these IN C-terminal mutants blocked infection mostly by affecting earlier steps of HIV-1 life cycle, such as reverse transcription and/or viral DNA nuclear import steps, which are different from the action of D64E mutant on viral DNA integration.

(21) Effect of IN mutants on viral infection in dividing and nondividing C8166 T cells. To further test whether these C-terminal mutants could induce similar phenotypes in CD4+ T cells, we infected dividing and non-dividing (aphidicolin-treated) C8166 CD4+ T cells with equal amounts of VSV-G pseudotyped IN mutant viruses (at 5 cpm of RT activity/cell). Since all IN mutant viruses contain a luciferase (luc) gene in place of the nef gene, viral infection can be monitored by using a sensitive luc assay which could efficiently detect viral gene expression from integrated and unintegrated viral DNA [Poon et al. J Virol. 2003; 77:3962-3972]. After 48 hours of infection, equal amounts of cells were lysed in 50 μl of luc lysis buffer and then, 10 μl of cell lysates was used for measurement of luc activity, as described in Materials and Methods. Results showed that the D64E mutant infection in dividing C8166 T cells induced 14.3×10⁴ RLU of luc activity (FIG. 17A), which was approximately 1000-fold lower than that in the wild type virus infection. This level of luc activity detected in D64E mutant infection is mostly due to nef gene expression from the unintegrated DNA. In agreement with the finding by MAGI assay described in FIG. 16, the Luc activity detected in KK215,9AA, KK240,4AE and RK263,4AA mutant samples were approximately 13%, 5% and 36% of level of D64E mutant infection (FIG. 17A). In parallel, infection of different IN mutants in non-dividing C8166 T cells was also evaluated and similar results were observed (FIG. 17B).

To test whether these IN mutants had similar effects during HIV-1 envelope-mediated single cycle infection, we produced virus stocks by co-transfecting 293T cells with a HIV-1 envelope-competent NLlucRI provirus with each Vpr-RT-IN mutant expresser. Then, dividing CD4+ C8166 cells were infected with each virus stock (at 10 cpm RT activity/cells). At 48 hours post-infection, cells were collected and measured for luc activity. Results from FIG. 4C showed that, similar to results obtained from VSV-G pseudotyped virus infection (FIG. 17A), the Luc activity detected in cells infected by HIV-1 envelope competent KK215,9AA, KK240,4AE and RK263,4AA mutant viruses were approximately 13.5%, 6% and 29% of level of D64E mutant infection (FIG. 17C). All of these results confirm the data from HeLa-CD4-CCR5-LTR-β-Gal infection (FIG. 16) by using either VSV-G- and HIV-1 envelope-mediated infections and suggest again that the significantly attenuated infection of KK215,9AA, KK240,4AE and RK263,4AA mutant viruses may be due to their defect(s) at reverse transcription and/or viral DNA nuclear import steps.

(22) Effects of different IN mutants on HIV-1 reverse transcription, DNA nuclear import, and proviral DNA integration. To directly assess the effect of IN C-terminal mutants KK215,9AA, KK240,4AE and RK263,4AA on each early step during viral infection, we analyzed the viral DNA synthesis, their nuclear translocation and integration following each IN mutant infection in dividing C8166 cells. Levels of HIV-1 late reverse transcription products were analyzed by semi-quantitative PCR after 12 hours of infection with HIV-1 specific 5′-LTR-U3/3′-Gag primers and Southern blot, as previously described [Ao et al. J Virol. 2004; 78:3170-3177 and Simon et al. J Virol. 1996; 70:5297-5305]. Also, intensity of amplified HIV-1 specific DNA in each sample was evaluated by laser densitometric scanning of bands in Southern blot autoradiograms (FIG. 18A). Results showed that total viral DNA synthesis in both KK215,9AA and RK263,4AA infection reached approximately 61% and 46% of that of the wild type (wt) virus infection (FIGS. 18A and 18B). Strikingly, in KK240,4AE sample, detection of viral DNA synthesis was drastically reduced, which only reached 21% of viral DNA level in WT sample (FIGS. 18A and 18B). These results indicate that all three C-terminal mutants negatively affected viral reverse transcription during viral infection and KK240,4AE mutant exhibited most profound effect.

Meanwhile, the nucleus- and cytoplasm-associated viral DNA levels were analyzed at 24 hours post-infection in C8166 T cells. The infected cells were first gently lysed and separated into nuclear and cytoplasmic fractions by using a previously described fractionation technique [Simon et al. J Virol. 1996; 70:5297-5305]. Then, levels of HIV-1 late reverse transcription products in each fraction were analyzed by semi-quantitative PCR, as described above. Results revealed differential effects of C-terminal mutants on HIV-1 DNA nuclear import. In the wt, D64E and RK263,4AA virus-infected samples, there were respectively 70%, 72% and 68% of viral DNA associated with nuclear fractions (FIG. 18C upper panel, lanes 1 and 2; 3 and 4; 9 and 10 and FIG. 18D). For KK240,4AE mutant, approximately 51% of viral DNA was nucleus-associated (FIG. 18C upper panel, lanes 7 and 8 and FIG. 18D). Remarkably, in KK215,9AA infected sample, viral cDNA was found predominantly in the cytoplasm and only approximately 21% of viral DNA was associated with the nuclear fraction (FIG. 18C upper panel, lanes 5 and 6; and FIG. 18D). Meanwhile, the integrity of fractionation procedure was validated by detection of β-globin DNA, which was found solely in the nucleus and levels of this nucleus-associated cellular DNA were similar in each nuclear sample (FIG. 18C, lower panel).

Even though the C-terminal mutants were shown to significantly affect HIV-1 reverse transcription and/or nuclear import, the various low levels of nucleus-associated viral DNA during the early stage of replication (FIG. 18C) may still be accessible for viral DNA integration. To address this question, 1×10⁶ dividing C8166 T cells were infected with equivalent amounts of each single cycle replicating virus stock (5 cpm/cell), as indicated in FIG. 6 and after 24 hours of infection, the virus integration level was checked by using a previously described sensitive Alu-PCR technique [Ao et al. J Virol. 2004; 78:3170-3177]. Results revealed that, while the wt virus resulted in an efficient viral DNA integration (FIG. 19, upper panel; lanes 1 and 2), there was no viral DNA integration detected in D64E mutant (lanes 3 to 4) and in all three C-terminal mutant infection samples (lanes 5 to 10), although similar levels of cellular β-globin gene were detected in each sample (FIG. 19, middle panel). These results suggest that, in addition to affecting HIV-1 reverse transcription and nuclear import, all three C-terminal IN mutants tested in this study also negatively affected viral DNA integration. Overall, all of these results indicate that all three IN C-terminal mutants belong to class II mutants, which affect different early steps during HIV-1 replication. Among these mutants, KK240,4AE showed the most profound inhibition on reverse transcription; KK215,9AA, and to a lesser extent KK240,4AE, impaired viral DNA nuclear translocation during early HIV-1 infection in C8166 T cells.

(23) Mutations in the C-terminal domain of IN inhibits HIV single-cycle replication and affect reverse transcription and nuclear import. To test the effect of the IN mutant (INKKRK) on HIV-1 replication, the mutant was introduced into a VSV-G pseudotyped HIV-1 single-cycle replication system (see Ao et al. 2004 J Virol. 78:3170-7). Briefly, the IN_{KKRK} mutant was first introduced into a CMV-Vpr-RT-IN expressor. Then, the VSV-G pseudotyped HIV-1 single cycle replicating virus (vKKRK) was produced in 293T cells by co-transfection with CMV-Vpr-RT-IN_{KKRK}, an RT/IN-deleted HIV provirus NLlucΔBgl/ΔRI and a VSV-G expressor. In parallel, the VSV-G pseudotyped wild type virus (vINwt) and IN class I mutant D64E virus (vD64E), were also produced in parallel as controls. After each virus stock was harvested, the trans-incorporation of RT and IN as well as the Gag composition in the viral particle was analyzed using western blot with a human anti-HIV positive serum. Results showed that similar amounts of RT, IN and Gagp24 were detected in each virus preparation (FIG. 12A). Then, equal amount of each virus stock (as adjusted by amounts of HIV-1 Gagp24) was used to infect CD4⁺ C8166 cells. At different time intervals, the luciferase (luc) activity in equal amounts of cells was measured, as shown in FIG. 12B. Since D64E mutant virus (vD64E) is unable to mediate viral DNA integration, its infection expressed very low luc activity (FIG. 12B). The luc activity detected from the vKKRK virus infection was considerably lower than that of the D64E mutant virus at different time points (FIG. 12B), indicating that the vKKRK virus lost its replication ability in CD4+ C8166 cells.

To test which step of the infection was affected in the IN mutants, the cytoplasm- and nucleus-associated viral DNA levels were analyzed at 24 hours post-infection, using semi-quantitative PCR and southern blot. For the vKKRK virus infection, the level of total viral DNA (including the cytoplasm- and nucleus-associated viral DNA levels) was reduced by approximately 60%, compared to the total viral DNA level detected from the wt virus infection (FIG. 12C, upper panel, compare lanes 5 and 6 to lanes 1 and 2, and D, left panel). Moreover, results indicated that for the wt and vD64E virus infections, approximately 73 and 77% of viral DNA were associated with nuclear fractions (FIG. 12C (upper panel, lanes 1 to 4) and D, right panel). However, during vKKRK infection, only 44% of viral DNA was nucleus-associated (FIG. 12C (upper panel, lanes 5 and 6) and D, right panel). The integrity of the fractionation procedure was also validated by detection of β-globin DNA, which was found solely in the nucleus, and levels of this cellular DNA were similar in each nuclear sample (FIG. 12C, lower panel). Taken together, all of these results indicate that the Imp7-binding defect mutant virus vKKRK was unable to replicate in C8166 cells and displayed impairment at both viral reverse transcription and nuclear import.

(24) Expression of HIV-1 integrase C-terminal domain in viral producer cells inhibits subsequent HIV-1 infection in HeLa-β-Gal-CD4-CCR5 cells and in CD4⁺ T-lymphoid MT4 cells.

It is possible that expression of the IN C-terminal domain in the late stage of HIV-1 replication inhibits HIV-1 infection. To test this, the IN C-terminal domain was co-transfected with NL4.3-Nef+/GFP+ provirus in 293T cells. After 48 hours of transfection, viruses were harvested and equal amounts of viruses (as measured by virion-associated reverse transcriptase activity (FIG. 20A)) were used to infect HeLa-β-Gal-CD4/CCR5 cells (FIG. 20B) or CD4+ lymphoid MT4 cells (FIG. 20C). Results showed that the virus infection was significantly impaired when HeLa-β-Gal-CD4/CCR5 cells or MT4 cells were infected by viruses which were produced by 293T cells expressing the C-terminal domain of IN (FIGS. 20B and 20C). The results suggest that expression of IN peptides derived from the C-terminal domain of IN during the late stage of HIV-1 replication will impair HIV-1 infection.

(25) SiRNA-mediated silencing of Imp7 inhibits HIV-1 infection. We investigated the effect of small interfering RNA (siRNA)-mediated Imp7-knockdown on HIV-1 replication. First, we tested the efficiency of Imp7 knockdown, the Imp7-siRNA (100 pmol) was introduced into 293T and HeLa-β-Gal-CD4/CCR5 cells once a day for two days (FIG. 13A). Briefly, 293T cells and HeLa-β-gal-CD4/CCR5 cells were plated at 2×10⁵ cells/well in 6-well plates and transfected at the next day with 100 pmol of Imp7-specific small interfering RNA (siRNA) duplex (IPO7-HSS116173) with Lipofectamine™ RNAiMAX Reagent (Invitrogen). After 18 h of first transfection, another Imp7 siRNA duplex (IPO7-HSS116174) was transfected again into cells. These two Imp7-siRNA duplexes (Stealth RNAi), IPO7-HSS116173 and IPO7-HSS116174, were synthesized by Invitrogen Inc and the targeting sequence are respectively corresponding to Imp7 mRNA nucleotides 1990-2013 (5′-UAAGCAGAUUCCCUCAAGCUGUUGG-3′; SEQ ID NO:21), and to Imp7 mRNA nucleotides 610-633 (sense 5′-AAUGCUGCAUUGCUGGCUACCAAUGG-3′; SEQ ID NO:22). In parallel, transfection of a scramble RNA (sc-RNA) (purchased from Santa Cruz Biotechnology) was used as control. At different time intervals, equal amounts of cells (0.5×10⁶ cells) were collected and monitored for Imp7 expression. Western blot results revealed that Imp7 protein expression were progressively decreased over the course of the experiments. At 48 hours following the first Imp7-siRNA transfection, the Imp7 protein level was reduced to approximately 30%, and at 96 hours, the level of Imp7 expression was reduced to <10% in both 293T and HeLa-β-Gal-CD4/CCR5 cells (FIG. 13B).

Next, we tested the effect of Imp7 knockdown on HIV-1 infection. To avoid the possibility that Imp7 might have effect on the late stage of viral replication and/or be packaged into viral particles and thus playing a role in subsequent viral infection, we first produced a VSV-G pseudotyped HIV-1 (NL4.3-BruΔBgl/luc+) from Imp7-siRNA- or scramble RNA (sc-RNA)-transfected 293T cells. The Imp7 protein expression in siRNA transfection cells was to <10% (at 96 hours of siRNA transfection) when the viruses were collected. Then, viruses (si-virus and sc-virus) produced from Im7-siRNA- or scRNA-transfected 293T cells were normalized by HIV Gagp24 levels and used to infect siRNA-treated and sc-RNA-treated HeLa-β-Gal-CD4/CCR5 cells (target cells) (FIG. 13A). Results in FIG. 7C showed that there were no significant luc activity differences detected in sc-RNA- and si-RNA-treated target cells after being infected with sc-virus (FIG. 13C, bars 1 and 2) or in the sc-RNA-treated cells being infected by si-virus (FIG. 7C, bar 3). However, when siRNA-treated HeLa-β-Gal-CD4/CCR5 cells were infected with si-virus, the luc activity was reduced to approximately 37% of the wt infection level (FIG. 13C, compare bar 4 to bar 1).

These observations were further extended to HIV-1 envelope-mediated viral infection. HIV-1 envelope competent si-HxBru and sc-HxBru viruses were produced in Imp7-siRNA and sc-RNA-treated HeLa-β-Gal-CD4/CCR5 cells by transfecting with a HIV-1 HxBru provirus and used to infect the Imp7-siRNA and sc-RNA-treated HeLa-β-Gal-CD4/CCR5 cells at 72 h post-transfection. The numbers of β-Gal positive cells were evaluated by MAGI assay at 48 h post infection. As expected, when Imp7-siRNA-treated HeLa-β-Gal-CD4/CCR5 cells were infected with si-virus, the β-Gal positive cell level was significantly reduced to approximately 27% of the wild type infection level (FIG. 13D, compare bar 4 to bar 1). Whereas, the β-Gal positive cell levels for sc-virus infection in siRNA-treated cells and for si-RNA virus infection in the control cells were slightly decreased to 76% and 70% of the wt infection level (FIG. 13D, compare bars 2 and 3 to bar 1). All of these results indicate that the knockdown of Imp7 in both HIV-1 producing and target cells impaired HIV-1 infection.

(26) Description of Terms and Expressions

The following definitions are provided as an aid to understanding the invention.

The terms “polypeptide,” “peptide” and “protein” are used herein to refer to a polymer of amino acid residues. The terms apply to amino acid polymers in which one or more amino acid residue is an analog or mimetic of a corresponding naturally occurring amino acid, as well as to naturally occurring amino acid polymers. The polypeptide sequences are displayed herein in the conventional N-terminal to C-terminal orientation. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

For amino acid and nucleic acid sequences, individual substitutions, deletions or additions that alter, add or delete an amino acid or nucleotide in the sequence create a variant sequence. A “conservatively modified variant” means the alteration results in the substitution of an amino acid with a chemically similar amino acid. Conservative substitution tables providing functionally similar amino acids are well known in the art. Such conservatively modified variants are in addition to and do not exclude polymorphic variants and alleles of the invention.

“Conservatively modified variants” applies to both peptide and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon in an amino acid herein, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. The following groups each contain amino acids that are conservative substitutions for one another:

### 1) Alanine (A), Glycine (G);

### 2) Serine (S), Threonine (T);

### 3) Aspartic acid (D), Glutamic acid (E);

### 4) Asparagine (N), Glutamine (Q);

### 5) Cysteine (C), Methionine (M);

### 6) Arginine (E), Lysine (K), Histidine (H);

### 7) Isoleucine (I), Leucine (L), Valine (V); and

### 8) Phenylalanine (F), Tyrosine (Y), Tryptophan (W).

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The phrases “coding sequence,” “structural sequence,” and “structural nucleic acid sequence” refer to a physical structure comprising an orderly arrangement of nucleic acids. The nucleic acids are arranged in a series of nucleic acid triplets that each form a codon. Each codon encodes for a specific amino acid. Thus, the coding sequence, structural sequence, and structural nucleic acid sequence encode a series of amino acids forming a protein, polypeptide, or peptide sequence. The coding sequence, structural sequence, and structural nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

The phrases “DNA sequence,” “nucleic acid sequence,” and “nucleic acid molecule” refer to a physical structure comprising an orderly arrangement of nucleic acids. The DNA sequence or nucleic acid sequence may be contained within a larger nucleic acid molecule, vector, or the like. In addition, the orderly arrangement of nucleic acids in these sequences may be depicted in the form of a sequence listing, figure, table, electronic medium, or the like.

The term “expression” refers to the transcription of a gene to produce the corresponding mRNA and translation of this mRNA to produce the corresponding gene product (i.e., a peptide, polypeptide, or protein).

The term “expression of antisense RNA” refers to the transcription of a DNA to produce a first RNA molecule capable of hybridizing to a second RNA molecule. Formation of the RNA-RNA hybrid inhibits translation of the second RNA molecule to produce a gene product.

The phrase “heterologous” refers to the relationship between two or more nucleic acid or protein sequences that are derived from different sources. For example, a promoter is heterologous with respect to a coding sequence if such a combination is not normally found in nature. In addition, a particular sequence may be “heterologous” with respect to a cell or organism into which it is inserted (i.e., does not naturally occur in that particular cell or organism). A heterologous sequence in a fusion protein means the heterologous sequence is different from the reference sequence (the reference sequence being the sequence of interest); e.g. a heterologous sequence in fusion with HIV integrase means the heterologous sequence is a sequence other than HIV integrase; a heterologous sequence in fusion with an antibody sequence (fragment or single chain antibody) means the heterologous sequence is not an antibody sequence. Usually the heterologous sequence is used to facilitate purification of the protein of interest (e.g. fusions with His tag allows purification using an antibody against the His tag), to facilitate monitoring of the protein of interest (e.g. fusions with a reporter sequence such as GFP or YFP), or to target the protein of interest (e.g. fusions with a signal sequence allows the protein to be directed to the secretory pathway; a membrane-translocating sequence mediates crossing of the membrane by the fusion protein.)

A “reporter sequence” refers to a nucleic acid or polypeptide of a gene product that can be expressed in the cell of interest and is used for ease of assay and detection. The reporter gene must be sufficiently characterized such that it can be operably linked to the promoter. Reporter genes used in the art include the LacZ gene from E. coli, the CAT gene from bacteria, the luciferase gene from firefly, the GFP gene from jellyfish, the YFP gene, galactose kinase (encoded by the galK gene), and β-glucosidase (encoded by the gus gene).

The phrase “operably linked” refers to the functional spatial arrangement of two or more nucleic acid regions or nucleic acid sequences. For example, a promoter region may be positioned relative to a nucleic acid sequence such that transcription of a nucleic acid sequence is directed by the promoter region. Thus, a promoter region is “operably linked” to the nucleic acid sequence.

The term “promoter” or “promoter region” refers to a nucleic acid sequence, usually found upstream (5′) to a coding sequence, that is capable of directing transcription of a nucleic acid sequence into mRNA. The promoter or promoter region typically provide a recognition site for RNA polymerase and the other factors necessary for proper initiation of transcription. As contemplated herein, a promoter or promoter region includes variations of promoters derived by inserting or deleting regulatory regions, subjecting the promoter to random or site-directed mutagenesis, etc. The activity or strength of a promoter may be measured in terms of the amounts of RNA it produces, or the amount of protein accumulation in a cell or tissue, relative to a promoter whose transcriptional activity has been previously assessed.

The term “recombinant vector” refers to any agent such as a plasmid, cosmid, virus, autonomously replicating sequence, phage, or linear single-stranded, circular single-stranded, linear double-stranded, or circular double-stranded DNA or RNA nucleotide sequence. The recombinant vector may be derived from any source; is capable of genomic integration or autonomous replication.

“Regulatory sequence” refers to a nucleotide sequence located upstream (5′), within, or downstream (3′) to a coding sequence. Transcription and expression of the coding sequence is typically impacted by the presence or absence of the regulatory sequence.

As used herein, the term “substantially purified” refers to a molecule separated from substantially all other molecules normally associated with it in its native state. More preferably a substantially purified molecule is the predominant species present in a preparation. The term “substantially purified” is not intended to encompass molecules present in their native state. Similarly, the term “isolated” refers to material, such as a nucleic acid or a protein, which is: (1) substantially or essentially free from components which normally accompany or interact with the material as found in its naturally occurring environment or (2) if the material is in its natural environment, the material has been altered by deliberate human intervention to a composition and/or placed at a locus in the cell other than the locus native to the material.

The term “recombinant” when used with reference, e.g., to a cell, or nucleic acid, protein, or vector, indicates that the cell, nucleic acid, protein or vector, has been modified from the natural state. Thus, for example, recombinant cells express genes that are not found within the native (naturally occurring) form of the cell or express a second copy of a native gene that is otherwise normally or abnormally expressed, under expressed or not expressed at all.

An “expressor” is a genetic construct to express a nucleic acid of interest. It is generated recombinantly or synthetically, with a series of specified nucleic acid elements that permit transcription of a the nucleic acid of interest in a host cell, and optionally integration or replication of the expresser in a host cell. The expresser can be part of a plasmid, virus, or nucleic acid fragment, of viral or non-viral origin. Typically, the expresser comprises a nucleic acid to be transcribed operably linked to a promoter.

(27) Integrase-Derived Peptides and Variants

The invention relates to peptides and variants derived from HIV-1 integrase. An example of an HIV-1 integrase is set out in SEQ ID NO:1 and FIG. 21 but others are known in the art. See Kuiken et al. HIV Sequence Compendium 2001. Los Alamos National Laboratory. 2001. Other HIV-1 IN sequences having variations in the C-terminal domain include sequences identified by NCBI accession numbers AAO61870, AAL01917, AAL01984, AAO61859, Q73368, AAO61895.

The peptides of the invention are based on the finding that certain specific regions of HIV-1 IN are important for HIV-1 replication and infection, possibly because these regions are important for nuclear translocation of the PIC into the nucleus. The regions of interest are found largely within amino acids 205-288 of IN, i.e. generally the C-terminal domain. Certain specific regions within the C-terminal domain are important and are generally within the tri-lysine proximal region, the tri-lysine distal region, and the arginine/lysine region. The peptides containing these regions are therefore useful at least as antagonists to the full-length, naturally occurring HIV-1 integrase. Without being bound by theory or mechanism, we think the peptides derived from these regions of the IN C-terminal domain compete with the naturally occurring IN, thereby inhibiting entry of HIV-1 PIC into the nucleus, and/or inhibiting assembly of the HIV-1 complex at early or late stages of infection.

In a similar manner to the IN-derived peptides, variants of IN can be used as antagonists to the full-length, naturally occurring HIV-1 integrase. Without being bound by theory or mechanism, we think the IN variants containing substitution or deletion mutations in the imp7-binding or impβ-binding regions of the IN C-terminal domain compete with the naturally occurring IN, thereby inhibiting assembly of the HIV-1 complex at early or late stages of infection.

(28) Synthesis and Recombinant Expression of IN-derived Peptides and Variants

Chemical synthesis, especially solid-phase synthesis may be used for short (e.g., less than 50 residues) peptides or those containing unnatural or unusual amino acids such as D-Tyr, ornithine, amino-adipic acid, and the like. Recombinant procedures are usually better for longer polypeptides.

Peptides can be synthesized chemically by such commonly used methods as t-BOC or FMOC protection of alpha-amino groups. Both methods involve stepwise syntheses whereby a single amino acid is added at each step starting from the carboxyl-terminus of the peptide (See, Coligan et al., Current Protocols in Immunology, Wiley Interscience, 1991, Unit 9). Peptides of the invention can also be synthesized by the solid phase peptide synthesis methods well known in the art. (Merrifield, J. Am. Chem. Soc., 85:2149, 1962), and Stewart and Young, Solid Phase Peptides Synthesis, Pierce, Rockford, Ill. (1984)). Peptides can be synthesized using a copoly(styrene-divinylbenzene) containing 0.1-1.0 mmol amines/g polymer. On completion of chemical synthesis, the peptides can be deprotected and cleaved from the polymer by treatment with liquid HF-10% anisole for about 0.25 to 1 hour at 0° C. After evaporation of the reagents, the peptides are extracted from the polymer with 1% acetic acid solution which is then lyophilized to yield the crude material.

The crude material can typically be purified by such techniques as gel filtration on Sephadex G-15 using 5% acetic acid as a solvent, by high pressure liquid chromatography, and the like. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and assessed by the solid phase Edman degradation. Automated synthesis using FMOC solid phase synthetic methods can be achieved using an automated peptide synthesizer.

“Amino acid cleavage site” refers to an amino acid or amino acids that serve as a recognition site for a chemical or enzymatic reaction such that the peptide chain is cleaved at that site by the chemical agent or enzyme. Amino acid cleavage sites include those at aspartic acid-proline (Asp-Pro), methionine (Met), tryptophan (Trp) or glutamic acid (Glu). “Acid-sensitive amino acid cleavage site” as used herein refers to an amino acid or amino acids that serve as a recognition site such that the peptide chain is cleaved at that site by acid. Particularly preferred is the Asp-Pro cleavage site which may be cleaved between Asp and Pro by acid hydrolysis.

Fusion polypeptides containing IN-derived sequences may contain a linking amino acid or amino acids for cleaving the specific IN-derived sequence from the polypeptide. For example, the IN-derived sequence may be produced as a fusion protein where the IN-derived sequence is fused to a heterologous polypeptide such as the commercially available His-tag, and where an amino acid cleavage site is placed between the IN-derived sequence and the heterologous peptide. The linking amino acid or amino acids are incorporated between the IN-derived sequence of interest and the remainder of the fusion in such a way that one or more cleavage reactions separate each polypeptide species to the degree necessary for intended applications.

As used herein, a fusion polypeptide is one that contains an IN-derived sequence fused at the N- or C-terminal end to a polypeptide unrelated to integrase, i.e. a heterologous polypeptide. A simple way to obtain such a fusion polypeptide is by translation of an in-frame fusion of the polynucleotide sequences, i.e., a hybrid gene. The hybrid gene encoding the fusion polypeptide is inserted into an expression vector which is used to transform or transfect a host cell. Alternatively, the polynucleotide sequence encoding the IN-derived sequence is inserted into an expressor in which the polynucleotide encoding the heterologous polypeptide is already present. Such vectors and instructions for their use are commercially available, e.g. the pMal-c2 or pMal-p2 system, in which the heterologous polypeptide is a maltose binding protein, the glutathione-5-transferase system, or the His-Tag system. These and other expression systems provide convenient means for further purification of the desired IN-derived sequence.

Amino acids that may be used to link the IN-derived sequence of interest to the remainder of the polypeptide include aspartic acid-proline, asparagine-glycine, methionine, cysteine, lysine-proline, arginine-proline, isoleucine-glutamic acid-glycine-arginine, and the like. Cleavage may be effected by exposure to the appropriate chemical reagent or cleaving enzyme. It should be recognized that cleavage may not be necessary for every IN-derived sequence or fusion polypeptide that is constructed. A cleavage site could be incorporated, or absent.

The invention also encompasses a method of producing a desired IN-derived sequence of high purity comprising the steps of transforming a compatible host with a vector suitable for expressing a fusion polypeptide containing the IN-derived sequence, culturing the host, isolating the fusion polypeptide by selective binding to an affinity matrix such as a carrier linked to an antibody specific for the heterologous polypeptide, and cleaving off the desired IN-derived sequence either directly from the carrier-bound fusion polypeptide or after desorption from the carrier.

A necessary condition to permit such cleavage of the produced polypeptide is that it contains a unique cleavage site which may be recognized and cleaved by suitable means. Such a cleavage site may be a unique amino-acid sequence recognizable by chemical or enzymatic means and located between the desired portion of the polypeptide and remainder of the fusion polypeptide to be produced. Such a specific amino acid sequence must not occur within the desired portion.

Examples of enzymatic agents include proteases, such as collagenase, which in some cases recognizes the amino acid sequence NH₂-Pro-X-Gly-Pro-COOH, wherein X is an arbitrary amino acid residue, e.g. leucine; chymosin (rennin), which cleaves the Met-Phe bond; kallikrein B, which cleaves on the carboxyl side of Arg in X-Phe-Arg-Y; enterokinase, which recognizes the sequence X-(Asp)ₙ-Lys-Y, wherein n=2-4, and cleaves it on the carboxyl side of Lys; thrombin which cleaves at specific arginyl bonds. Examples of chemical agents include cyanogen bromide (CNBr), which cleaves after Met; hydroxylamine, which cleaves the Asn-Z bond, wherein Z may be Gly, Leu or Ala; formic acid, which in high concentration (about 70%) specifically cleaves Asp-Pro. Thus, if the desired portion does not contain any methionine sequences, the cleavage site may be a methionine group which can be selectively cleaved by cyanogen bromide. Chemical cleaving agents may be preferred in certain cases because protease recognition sequences may be sterically hindered in the produced polypeptide.

The techniques for introducing DNA sequences coding for such amino acid cleavage sites into the DNA sequence coding for the polypeptide are well-known in the art.

As mentioned above, cleavage may be effected either with the fusion polypeptide bound to the affinity matrix or after desorption therefrom. A batch-wise procedure may be carried out as follows. The carrier having the fusion polypeptide bound thereto, e.g. IgG-Sepharose where the IgG is specific against the heterologous polypeptide, is washed with a suitable medium and then incubated with the cleaving agent, such as protease or cyanogen bromide. After removal of the carrier material having the heterologous polypeptide bound thereto, a solution containing the cleaved desired polypeptide and the cleavage agent is obtained, from which the former may be isolated and optionally further purified by techniques known in the art such as gel filtration, ion-exchange etc.

Where the fusion polypeptide comprises a protease recognition site, the cleavage procedure may be performed in the following way. The affinity matrix-bound fusion polypeptide is washed with a suitable medium, and then eluted with an appropriate agent which is as gentle as necessary to preserve the desired IN-derived sequence. Such an agent may, depending on the particular IN-derived sequence, be a pH-lowering agent such as a glycine buffer. The eluate containing the pure fusion polypeptide is then passed through a second column comprising the immobilized protease, e.g. collagenase when the cleavage site is a collagenase susceptible sequence. When passing therethrough the fusion polypeptide is cleaved into the desired IN-derived sequence and the heterologous polypeptide. The resulting solution is then passed through the same affinity matrix, or a different affinity matrix, to adsorb the heterologous polypeptide portion of the solution.

Recombinant procedures may be used to produce longer IN-derived peptides and variants. Expression system vectors, which incorporate the necessary regulatory elements for protein expression, as well as restriction endonuclease sites that facilitate cloning of the desired sequences into the vector, are known to those of skill in the art. A number of these expression vectors are commercially available, e.g. pGEX-3X (Amersham Pharmacia, Piscataway N.J.) which comprises a nucleotide sequence encoding a fusion protein including glutathione-5-transferase.

Alternately, cell-free systems known to those of skill in the art can be chosen for expression of the desired IN-derived sequence.

The purified IN-derived peptide, variant and fusion produced by the expresser system or by chemical synthesis can then be administered to the target cell, where the membrane-translocating sequence mediates the import of the fusion protein through the cell membrane of the target cell into the interior of the cell.

An expresser system can be chosen from among a number of such systems that are known to those of skill in the art. In one embodiment of the invention, the fusion protein can be expressed in Escherichia coli. In alternate embodiments of the present invention, fusion proteins may be expressed in other bacterial expression systems, viral expression systems, eukaryotic expression systems, or cell-free expression systems. Cellular hosts used by those of skill in the art include, but are not limited to, Bacillus subtilis, yeast such as Saccharomyces cerevisiae, Saccharomyces carlsbergenesis, Saccharomyces pombe, and Pichia pastoris, as well as mammalian cells such as 3T3, HeLa, and Vero. The expression vector chosen by one of skill in the art will include promoter elements and other regulatory elements appropriate for the host cell or cell-free system in which the fusion protein will be expressed. In mammalian expression systems, for example, suitable expression vectors can include DNA plasmids, DNA viruses, and RNA viruses. In bacterial expression systems, suitable vectors can include plasmid DNA and bacteriophage vectors.

Examples of specific expression vector systems include the pBAD/gIII vector (Invitrogen, Carlsbad, Calif.) system for protein expression in E. coli, which is regulated by the transcriptional regulator AraC. Dose-dependent induction enables identification of optimal expression conditions for the specific target protein to be expressed. By inserting the polynucleotide sequence of the membrane translocating sequence of the present invention either 5′ or 3′ to the polynucleotide sequence of a target protein, this vector can be used to express a number of fusion proteins for which optimal expression conditions may vary. Furthermore, the vector encodes the polyhistidine (6×His) sequence and an epitope tag to allow rapid purification of the fusion protein with a nickel-chelating resin, along with protein detection with specific antibodies to detect the presence of the secreted protein.

An example of a vector for mammalian expression is the pcDNA3.1/V5-His-TOPO eukaryotic expression vector (Invitrogen). In this vector, the fusion protein can be expressed at high levels under the control of a strong cytomegalovirus (CMV) promoter. A C-terminal polyhistidine (6×His) tag enables fusion protein purification using nickel-chelating resin. Secreted protein produced by this vector can be detected using an anti-His (C-term) antibody.

A baculovirus expression system can also be used for production of the IN-derived peptide, variant and fusion. A commonly used baculovirus is AcMNPV. Cloning of the MTS/target protein DNA can be accomplished by using homologous recombination. The MTS/target protein DNA sequence is cloned into a transfer vector containing a baculovirus promoter flanked by baculovirus DNA, particularly DNA from the polyhedrin gene. This DNA is transfected into insect cells, where homologous recombination occurs to insert the MTS/target protein into the genome of the parent virus. Recombinants are identified by altered plaque morphology.

Proteins as described above can also be produced in the method of the present invention by mammalian viral expression systems. The Sindbis viral expression system, for example, can be used to express the fusion protein at high levels, such as pSinHis (Invitrogen, Carlsbad, Calif.). In vitro transcribed RNA molecules encoding the IN-derived peptide, variant and fusion, and the Sindbis proteins required for in vivo RNA amplification can be electroporated into baby hamster kidney (BHK) cells using methods known to those of skill in the art. Alternatively, the RNA encoding the fusion protein and Sindbis proteins required for in vivo RNA amplification can be cotransfected with helper RNA that permits the production of recombinant viral particles. Viral particles containing genetic material encoding the IN-derived peptide, variant and fusion can then be used to infect cells of a wide variety of cell types, including mammalian, avian, reptilian, and Drosophila.

An ecdysone-inducible mammalian expression system (Invitrogen, Carlsbad, Calif.) can also be used to express the IN-derived peptide, variant and fusion. With the ecdysone-inducible system, higher levels of protein production can be achieved by use of the insect hormone 20-OH ecdysone to activate gene expression via the ecdysone receptor.

Yeast host cells, such as Pichia pastoris, can also be used for the production of a IN-derived peptide, variant and fusion by the method of the present invention. Expression of proteins from plasmids transformed into Pichia has previously been described. Vectors for expression in Pichia are commercially available as part of a Pichia Expression Kit (Invitrogen, Carlsbad, Calif.).

Purification of heterologous protein produced in Pichia has been described in U.S. Pat. No. 5,004,688, and techniques for protein purification from yeast expression systems are well known to those of skill in the art. In the Pichia system, commercially available vectors can be chosen from among those that are more suited for the production of cytosolic, non-glycosylated proteins and those that are more suited for the production of secreted, glycosylated proteins, or those directed to an intracellular organelle, so that appropriate protein expression can be optimized for the target protein of choice.

The IN-derived peptides and variants may be in the form of fusion proteins which can contain cellular targeting tags for directing the agent to the cell membrane or cellular organelles e.g. the nucleus. Such tags can be used to mediate crossing of the membrane by the fusion protein. Suitable protein uptake tags include, for example and without limitation:

(1) poly-arginine and related peptoid tags (Chen et al. (2001) Chem. Biol. 8: 1123-1129, Wender et al. (2000) Proc. Natl. Acad. Sci. 97: 13003-13008);

(2) HIV TAT protein, its Protein Transduction Domain (PTD) spanning approximately amino acids 47-57, or synthetic analogs of the PTD (Becker-Hapak et al (2001) Methods 24: 247-256, Ho et al. (2001) Cancer Res. 61: 474-477);

(3) Drosophila Antennapedia protein, the domain spanning approximately amino acids 43-58 also called Helix-3 or Penetratin-1, or their synthetic analogs (Derossi et al (1998) Trends Cell Biol. 8: 84-87, Prochiantz (1996) Curr. Opin. Neurobiol. 6: 629-63);

(4) Herpesvirus VP22 protein, the domain spanning approximately amino acids 159-301, or portions or synthetic analogs thereof (Normand et al (2001) J. Biol. Chem. 276: 15042-15050, Phelan et al (1998) Nat. Biotech. 16: 440-443);

(5) Membrane-Translocating Sequence (MTS) from Kaposi fibroblast growth factor or related amino acid sequences (Rojas et al (1998) Nat. Biotech. 16: 370-375, Du et al (1998) J. Peptide Res. 51: 235-243);

(6) Pep-1, MPG, and similar peptides (Morris et al. (2001) Nat. Biotech. 19: 1173-1176, Morris et al. (1999) Nuc. Acid. Res. 27: 3510-3517);

(7) Transportan, Transportan 2, and similar peptides (Pooga et al. (1998) FASEB J. 12: 67-77; Pooga et al. (1998) Ann. New York Acad. Sci. 863: 450-453);

(8) Amphipathic model peptide and related peptide sequences (Scheller et al. (2000) Eur. J. Biochem. 267: 6043-6049, Scheller et al. (1999) J. Pept. Sci. 5: 185-194);

(9) Tag protein to be delivered with approximately amino acids 1-254 of Bacillus anthracis lethal factor (LF), and administer along with B. anthracis protective antigen (PA) to deliver the tagged protein into cells, or similar methods (Leppla et al (1999) J. App. Micro. 87: 284, Goletz et al. (1997) Proc. Natl. Acad. Sci. 94: 12059-12064); and

(10) Folic acid (Leamon and Low (2001) Drug Discov. Today 6: 44-51, Leamon et al (1999) J. Drug Targeting 7: 157-169).

Methods for attaching uptake tags to the proteins employ standard methods and will be recognized by one of skill in the art.

Examples of IN-derived peptides fused to the TAT membrane-translocating sequence are shown in FIG. 22.

Suitable conditions for protein import into the cell mediated by the membrane-translocating peptide of the present invention may include, as previously reported with NIH3T3 cells, incubating the cells in an extracellular concentration of fusion protein in the 20 μM range at 37° C. for 30 minutes, to accomplish the import of approximately 0.5-1×10⁶ molecules of transported protein per cell. Effective concentrations, however, may vary with differing proteins and cell types, and may be considered as amounts sufficient to result in import of fusion proteins into the cell, with protein import exhibiting dose-dependence. Methods for providing sufficient concentration to achieve protein import are known to those of skill in the art. Suitable import temperatures include temperatures in a preferred range between 22° C. and 37° C.

The IN-derived sequences produced by the method of the present invention may be administered in vitro by any of the standard methods known to those of skill in the art, such as addition of fusion protein to culture medium, or other known methods. Furthermore, the IN-derived peptide, variant and fusions produced by this method may be delivered in vivo by standard methods utilized for protein/drug delivery.

Administration of IN-derived peptide, variant and fusions produced by the method of the present invention may be performed for a time length of from 30 minutes to 18 hours, particularly when administration is accomplished by addition of IN-derived peptide, variant and fusions to culture media for in vitro use. For in vivo or in vitro use, effective administration time for a fusion protein produced by the method of the present invention may be readily determined by one of skill in the relevant art.

(29) Antibodies

The peptides of the invention are based on the regions largely within amino acids 205-288 of IN, i.e. generally the C-terminal domain, that are important for HIV-1 replication and infection. Thus, encompassed by the invention are antibodies that are specifically reactive against these specific regions; i.e. these regions constitute the antigenic epitopes. Such antibodies specifically bind to the respective polypeptides via the antigen-binding sites of the antibody (as opposed to non-specific binding). Thus, the IN-derived peptides, variants and fusions as set forth above may be employed as “immunogens” in producing antibodies immunoreactive therewith, and specifically react with the regions of the IN C-terminal domain that interact with imp7 or impβ. Some of these antibodies, by their specific binding to IN at the regions important for interaction with imp7 and impβ, should inhibit IN binding to imp7 and impβ, thereby inhibiting HIV-1 proliferation. Such antibodies immunoreactive against the IN C-terminal regions which define the reactive epitope are in isolated form and are not intended to encompass antibodies that may have been raised against integrase in the past, either as a means to obtain an antibody for research or commercial use, or in individuals infected with HIV.

An antibody of the invention is either polyclonal or monoclonal. Monospecific antibodies may be recombinant, e.g., chimeric (e.g., constituted by a variable region of murine origin associated with a human constant region), humanized (a human immunoglobulin constant backbone together with hypervariable region of animal, e.g., murine, origin), and/or single chain. Both polyclonal and monospecific antibodies may also be in the form of immunoglobulin fragments, e.g., F(ab)′2 or Fab fragments. Both polyclonal and monoclonal antibodies may be prepared by conventional techniques.

Hybridoma cell lines that produce monoclonal antibodies specific for the polypeptides of the invention are also contemplated herein. Such hybridomas may be produced and identified by conventional techniques. One method for producing such a hybridoma cell line comprises immunizing an animal with a polypeptide or a DNA encoding a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. The monoclonal antibodies may be recovered by conventional techniques.

The monoclonal antibodies of the present invention include chimeric antibodies, e.g., humanized versions of murine monoclonal antibodies. Such humanized antibodies may be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment may comprise the antigen binding site of a murine monoclonal antibody and a variable region fragment (lacking the antigen-binding site) derived from a human antibody. Procedures for the production of chimeric and further engineered monoclonal antibodies are known in the art.

Antigen-binding fragments of the antibodies, which may be produced by conventional techniques, are also encompassed by the present invention. Examples of such fragments include, but are not limited to, Fab and F(ab′)2 fragments. Antibody fragments and derivatives produced by genetic engineering techniques are also provided.

In one embodiment, the antibodies are specific for epitopes defining the imp7 and impβ binding regions within the C-terminal domain of HIV-1 integrase and do not cross-react with other proteins. Screening procedures by which such antibodies may be identified are well known, and may involve immunoaffinity chromatography, for example.

The antibodies of the invention can be used in assays to detect the presence of the polypeptides or fragments of the invention, either in vitro or in vivo. The antibodies also may be employed in purifying polypeptides or fragments of the invention by immunoaffinity chromatography.

In one embodiment of the invention, antibodies to epitopes defining the imp7 and impβ binding regions within the C-terminal domain of HIV-1 integrase are antagonistic, that is, they bind to the specific regions within IN and prevent the binding of a receptor to IN, specifically imp7 and impβ. Such antagonistic antibodies would be useful for inhibiting HIV-1 proliferation.

The antibodies of the invention are produced and identified using standard immunological assays, e.g., Western blot analysis, dot blot assay, or ELISA (see, e.g., Coligan et al., Current Protocols in Immunology (1994) John Wiley & Sons, Inc., New York, N.Y.). The antibodies are used in diagnostic methods to detect the presence of IN in a sample, such as a biological sample. The antibodies are also used in affinity chromatography for purifying a polypeptide or polypeptide derivative of IN.

Briefly, for making monoclonal antibodies, somatic cells from the a host animal immunized with antigen, with potential for producing antibody, are fused with myeloma cells, forming a hybridoma of two cells by conventional protocol. Somatic cells may be derived from the spleen, lymph node, and peripheral blood of transgenic mammals.

Somatic cell-myeloma cell hybrids are plated in multiple wells with a selective medium, such as HAT medium. Selective media allow for the detection of antibodyproducing hybridomas over other undesirable fused-cell hybrids. Selective media also prevent growth of unfused myeloma cells which would otherwise continue to divide indefinitely, since myeloma cells lack genetic information necessary to generate enzymes for cell growth. B lymphocytes derived from somatic cells contain genetic information necessary for generating enzymes for cell growth but lack the “immortal” qualities of myeloma cells, and thus, last for a short time in selective media. Therefore, only those somatic cells which have successfully fused with myeloma cells grow in the selective medium. The infused cells were killed off by the HAT or selective medium.

A screening method can be used to isolate potential antibodies derived from hybridomas grown in the multiple wells, where the antibodies are specifically reactive with the IN-derived peptides described herein. Multiple wells are used in order to prevent individual hybridomas from overgrowing others. Screening methods used to identify such antibodies include enzyme immunoassays, radioimmunoassays, plaque assays, cytotoxicity assays, dot immunobinding assays, fluorescence activated cell sorting (FACS), and other in vitro binding assays.

Hybridomas which test positive for antibodies having the desired immunoreactivity are maintained in culture and may be cloned in order to produce monoclonal antibodies. Alternatively, desired hybridomas can be injected into a histocompatible animal of the type used to provide the somatic and myeloma cells for the original fusion. The injected animal develops tumors secreting the specific monoclonal antibody produced by the hybridoma.

The monoclonal antibodies secreted by the selected hybridoma cells are suitably purified from cell culture medium or ascites fluid by conventional immunoglobulin purification procedures such as, for example, protein A-SEPHAROSE hydroxylapatite chromatography, gel electrophoresis, dialysis, or affinity chromatography.

Contemplated within the scope of the invention are single chain antibodies that are specifically reactive with the imp7 or impβ binding regions of IN. Specifically contemplated are single chain antibodies fused to the cellular targeting tags and sequences described above, especially the TAT membrane-translocating sequence, to facilitate uptake of the single chain antibody into the cell.

Single chain antibodies are formed by linking the heavy and light chain fragments of the Fv region via an amino acid bridge, resulting in a single chain polypeptide. To create a single chain antibody (scFv), the VH- and VL-encoding DNA fragments are operably linked to another fragment encoding a flexible linker, e.g., encoding the amino acid sequence (Gly₄-Ser) 3, such that the VH and VL sequences can be expressed as a contiguous single-chain protein, with the VL and VH regions joined by the flexible linker. The single chain antibody may be monovalent, if only a single VH and VL are used, bivalent, if two VH and VL are used, or polyvalent, if more than two VH and VL are used.

Recombinant expression of an antibody, or fragment, derivative or analog thereof, (e.g., a heavy or light chain of an antibody or a single chain antibody), requires construction of an expresser containing a polynucleotide that encodes the antibody. Once a polynucleotide encoding an antibody molecule or a heavy or light chain of an antibody, or portion thereof (preferably containing the heavy or light chain variable domain) has been obtained, the vector for the production of the antibody molecule may be produced by recombinant DNA technology using techniques well known in the art. Thus, methods for preparing a protein by expressing a polynucleotide encoding the desired polypeptide as described above are applicable for recombinant expression of monoclonal and single chain antibodies. Methods which are well known to those skilled in the art can be used to construct expressors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule and the variable domain of the antibody may be cloned into such a vector for expression of the entire heavy or light chain.

Antibody fragments and single chain antibodies specific against the IN-derived sequences may be produced recombinantly as fusions with heterologous sequences. Such antibody fusions can be produced as described above for fusion polypeptides. In particular, the antibody fragments and single chain antibodies may be in the form of fusion proteins which can contain cellular targeting tags for directing the antibody fragments and single chain antibodies to the cell membrane or cellular organelles. As described above, suitable protein uptake tags include for example poly-arginine and related peptoid tags, HIV TAT protein, its Protein Transduction Domain (PTD) spanning approximately amino acids 47-57, or synthetic analogs of the PTD; Drosophila Antennapedia protein, the domain spanning approximately amino acids 43-58 also called Helix-3 or Penetratin-1, or their synthetic analogs; herpesvirus VP22 protein, the domain spanning approximately amino acids 159-301, or portions or synthetic analogs thereof; membrane-translocating sequence (MTS) from Kaposi fibroblast growth factor or related amino acid sequences; Pep-1, MPG, and similar peptides; Transportan, Transportan 2, and similar peptides; amphipathic model peptide and related peptide sequences; Tag protein to be delivered with approximately amino acids 1-254 of Bacillus anthracis lethal factor (LF), and administer along with B. anthracis protective antigen (PA) to deliver the tagged protein into cells; and Folic acid. It is noted that conjugation of mouse IgG at the Fc domain to a 17-mer synthetic peptide incorporating the membrane-translocating (MTS) and nuclear import sequence of HIV-1 TAT promoted internalization and nuclear uptake of the IgG, and that immunoreactivity was preserved in the tat-MTS-IgG fusion. See Hu et al 2006 Eur J Nuclear Med Molec Imaging 33(3):301-310, the sections relating to cell membrane import incorporated herein by reference.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies that bind to a Therapeutic protein and that may correspond to a Therapeutic protein portion of an albumin fusion protein of the invention or fragments thereof can be fused to heterologous polypeptide sequences described herein or otherwise known in the art, to facilitate purification.

(30) Anti-sense Sequences

The inventors found that certain specific regions of HIV-1 IN are important for HIV-1 replication and infection. The regions of interest are found largely within amino acids 205-288 of IN. It is contemplated that providing antisense molecules and ribozymes to degrade and/or inhibit translation of IN mRNA specifically at these regions, proliferation of HIV-1 will be inhibited.

Therefore, in alternative embodiments, the invention provides antisense molecules and ribozymes, targeted to the C-terminal domain of IN, specifically within the sequences encoding amino acids 205-288 of IN and the imp7 and impβ binding regions, for exogenous administration to effect the degradation and/or inhibition of the translation of IN mRNA.

Examples of therapeutic antisense oligonucleotide applications, incorporated herein by reference, include: U.S. Pat. No. 5,135,917, issued Aug. 4, 1992; U.S. Pat. No. 5,098,890, issued Mar. 24, 1992; U.S. Pat. No. 5,087,617, issued Feb. 11, 1992; U.S. Pat. No. 5,166,195 issued Nov. 24, 1992; U.S. Pat. No. 5,004,810, issued Apr. 2, 1991; U.S. Pat. No. 5,194,428, issued Mar. 16, 1993; U.S. Pat. No. 4,806,463, issued Feb. 21, 1989; U.S. Pat. No. 5,286,717 issued Feb. 15, 1994; U.S. Pat. No. 5,276,019 and U.S. Pat. No. 5,264,423; BioWorld Today, Apr. 29, 1994, p. 3.

Preferably, in antisense molecules, there is a sufficient degree of complementarity to mRNA encoding amino acids 205-288 of IN and the imp7 and impβ binding regions, to avoid non-specific binding of the antisense molecule to non-target sequences under conditions in which specific binding is desired, such as under physiological conditions in the case of in vivo assays or therapeutic treatment or, in the case of in vitro assays, under conditions in which the assays are conducted. The target mRNA for antisense binding may include not only the information to encode a protein, but also associated ribonucleotides, which for example form the 5′-untranslated region, the 3′-untranslated region, the 5′ cap region and intron/exon junction ribonucleotides. A method of screening for antisense and ribozyme nucleic acids that may be used to provide such molecules as Shc inhibitors of the invention is disclosed in U.S. Pat. No. 5,932,435 (which is incorporated herein by reference).

Antisense molecules (oligonucleotides) of the invention may include those which contain intersugar backbone linkages such as phosphotriesters, methyl phosphonates, short chain alkyl or cycloalkyl intersugar linkages or short chain heteroatomic or heterocyclic intersugar linkages, phosphorothioates and those with CH₂—NH—O—CH₂, CH₂—N(CH₃)—O—CH₂ (known as methylene(methylimino) or MMI backbone), CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Oligonucleotides having morpholino backbone structures may also be used (U.S. Pat. No. 5,034,506). In alternative embodiments, antisense oligonucleotides may have a peptide nucleic acid (PNA, sometimes referred to as “protein nucleic acid”) backbone, in which the phosphodiester backbone of the oligonucleotide may be replaced with a polyamide backbone wherein nucleosidic bases are bound directly or indirectly to aza nitrogen atoms or methylene groups in the polyamide backbone (Nielsen et al., 1991, Science 254:1497 and U.S. Pat. No. 5,539,082). The phosphodiester bonds may be substituted with structures which are chiral and enantiomerically specific. Persons of ordinary skill in the art will be able to select other linkages for use in practice of the invention.

Oligonucleotides may also include species which include at least one modified nucleotide base. Thus, purines and pyrimidines other than those normally found in nature may be used. Similarly, modifications on the pentofuranosyl portion of the nucleotide subunits may also be effected. Examples of such modifications are 2′-O-alkyl- and 2′-halogen-substituted nucleotides. Some specific examples of modifications at the 2′ position of sugar moieties which are useful in the present invention are OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. One or more pentofuranosyl groups may be replaced by another sugar, by a sugar mimic such as cyclobutyl or by another moiety which takes the place of the sugar.

In some embodiments, the antisense oligonucleotides in accordance with this invention may comprise from about 5 to about 100 nucleotide units. As will be appreciated, a nucleotide unit is a base-sugar combination (or a combination of analogous structures) suitably bound to an adjacent nucleotide unit through phosphodiester or other bonds forming a backbone structure.

(31) siNA

The inventors found that certain specific regions of HIV-1 IN are important for HIV-1 replication and infection. The regions of interest are found largely within amino acids 205-288 of IN. It is contemplated that providing antisense molecules and ribozymes to degrade and/or inhibit translation of IN mRNA specifically at these regions, proliferation of HIV-1 will be inhibited. Therefore it is contemplated that expression of integrase may be inhibited or prevented using RNA interference (RNAi) targeted at the specific regions of IN identified by the inventors. RNAi may be used to create a pseudo “knockout”, i.e. a system in which the expression of the product encoded by a gene or coding region of interest is reduced, resulting in an overall reduction of the activity of the encoded product in a system. As such, RNAi may be performed to target a nucleic acid of interest or fragment or variant thereof, to in turn reduce its expression and the level of activity of the product which it encodes. Such a system may be used for functional studies of the product, as well as to treat disorders related to the activity of such a product. RNAi is described in for example US publication 20050191618, herein incorporated by reference. Reagents and kits for performing RNAi are available commercially from for example Ambion Inc. (Austin, Tex., USA) and New England Biolabs Inc. (Beverly, Mass., USA).

The initial agent for RNAi in some systems is thought to be dsRNA molecule corresponding to a target nucleic acid. The dsRNA is then thought to be cleaved into short interfering RNAs (siRNAs) which are 21-23 nucleotides in length (19-21 bp duplexes, each with 2 nucleotide 3′ overhangs). The enzyme thought to effect this first cleavage step has been referred to as “Dicer” and is categorized as a member of the RNase III family of dsRNA-specific ribonucleases. Alternatively, RNAi may be effected via directly introducing into the cell, or generating within the cell by introducing into the cell a suitable precursor (e.g. vector encoding precursor(s), etc.) of such an siRNA or siRNA-like molecule. An siRNA may then associate with other intracellular components to form an RNA-induced silencing complex (RISC). The RISC thus formed may subsequently target a transcript of interest via base-pairing interactions between its siRNA component and the target transcript by virtue of homology, resulting in the cleavage of the target transcript approximately 12 nucleotides from the 3′ end of the siRNA. Thus the target mRNA is cleaved and the level of protein product it encodes is reduced.

RNAi may be effected by the introduction of suitable in vitro synthesized siRNA or siRNA-like molecules into cells. RNAi may for example be performed using chemically-synthesized RNA. Alternatively, suitable expression vectors may be used to transcribe such RNA either in vitro or in vivo. In vitro transcription of sense and antisense strands (encoded by sequences present on the same vector or on separate vectors) may be effected using for example T7 RNA polymerase, in which case the vector may comprise a suitable coding sequence operably-linked to a T7 promoter. The in vitro-transcribed RNA may in embodiments be processed (e.g. using E. coli RNase III) in vitro to a size conducive to RNAi. The sense and antisense transcripts are combined to form an RNA duplex which is introduced into a target cell of interest. Other vectors may be used, which express small hairpin RNAs (shRNAs) which can be processed into siRNA-like molecules. Various methods for introducing such vectors into cells, either in vitro or in vivo are known in the art.

Accordingly, in an embodiment integrase expression may be inhibited by introducing into or generating within a cell an siRNA or siRNA-like molecule corresponding to a nucleic acid encoding the C-terminal domain of IN (amino acids 205-288) or imp7/impβ-binding fragments thereof, or to an nucleic acid homologous thereto. “siRNA-like molecule” refers to a nucleic acid molecule similar to an siRNA (e.g. in size and structure) and capable of eliciting siRNA activity, i.e. to effect the RNAi-mediated inhibition of expression. In various embodiments such a method may entail the direct administration of the siRNA or siRNA-like molecule into a cell, or use of the vector-based methods described above. In an embodiment, the siRNA or siRNA-like molecule is less than about 30 nucleotides in length. In a further embodiment, the siRNA or siRNA-like molecule is about 21-23 nucleotides in length. In an embodiment, siRNA or siRNA-like molecule comprises a 19-21 bp duplex portion, each strand having a 2 nucleotide 3′ overhang. In embodiments, the siRNA or siRNA-like molecule is substantially identical to a nucleic acid encoding the C-terminal domain of IN (amino acids 205-288) or imp7/impβ-binding fragments thereof. In embodiments, the sense strand of the siRNA or siRNA-like molecule is substantially identical to a sequence found in HIV-1 which encodes SEQ ID NO:2 or a fragment thereof (RNA having U in place of T residues of the DNA sequence).

A siNA of the invention can be unmodified or chemically-modified. It can be chemically synthesized, expressed from a vector or enzymatically synthesized. The use of chemically-modified siNA improves various properties of native siNA molecules through increased resistance to nuclease degradation in vivo and/or through improved cellular uptake.

A siNA molecule can comprise a sense region and an antisense region, where the antisense region comprises sequence complementary to a HIV RNA sequence and the sense region comprises sequence complementary to the antisense region. A siNA molecule can be assembled from two nucleic acid fragments wherein one fragment comprises the sense region and the second fragment comprises the antisense region of said siNA molecule. The sense region and antisense region can be connected via a linker molecule, including covalently connected via the linker molecule. The linker molecule can be a polynucleotide linker or a non-nucleotide linker.

The siNA may be a double-stranded molecule that down-regulates expression of the HIV-1 IN gene, wherein the siNA molecule comprises about 15 to about 28 base pairs. The siNA molecule may comprise a first and a second strand; each strand of the siNA molecule is about 18 to about 28 nucleotides in length; the first strand of the siNA molecule comprises nucleotide sequence having sufficient complementarity to the HIV RNA for the siNA molecule to direct cleavage of the HIV RNA via RNA interference; and the second strand of said siNA molecule comprises nucleotide sequence that is complementary to the first strand. The siNA molecule may comprise no ribonucleotides, or comprise one or more ribonucleotides. One strand of the double-stranded siNA molecule may comprise a nucleotide sequence that is complementary to a nucleotide sequence of the HIV IN gene at the C-terminal domain, and a second strand of the double-stranded siNA molecule may comprise a nucleotide sequence substantially similar to the nucleotide sequence or a portion of the HIV IN RNA.

In some embodiments, each strand of the siNA molecule may comprise about 18 to about 23 nucleotides, and wherein each strand comprises at least about 19 nucleotides that are complementary to the nucleotides of the other strand. The siNA molecule may also comprise an antisense region comprising a nucleotide sequence that is complementary to a nucleotide sequence of the HIV IN gene at the C-terminal domain, and further comprising a sense region; the sense region may comprise a nucleotide sequence substantially similar to the nucleotide sequence of the HIV IN gene at the C-terminal domain or a portion thereof. The sense region may be connected to the antisense region via a linker molecule which may be a polynucleotide linker or a non-nucleotide linker. The pyrimidine nucleotides in the sense region may be 2′-O-methylpyrimidine nucleotides, and the purine nucleotides in the sense region may be 2′-deoxy purine nucleotides. The pyrimidine nucleotides present in the sense region may also be 2′-deoxy-2′-fluoro pyrimidine nucleotides.

In some embodiments, the siNA molecule comprises a sense region and an antisense region. The antisense region comprises a nucleotide sequence that is complementary to a nucleotide sequence of RNA encoded by the HIV IN gene at the C-terminal domain, or a portion thereof, and the sense region comprises a nucleotide sequence that is complementary to the antisense region.

In some embodiments, the siNA molecule is assembled from two separate oligonucleotide fragments. One fragment comprises the sense region and a second fragment comprises the antisense region of the siNA molecule. The fragment comprising the sense region may include a terminal cap moiety at a 5′-end, a 3′-end, or both of the 5′ and 3′ ends of the fragment comprising the sense region. The terminal cap moiety may be an inverted deoxy abasic moiety. The pyrimidine nucleotides of the antisense region may be 2′-deoxy-2′-fluoro pyrimidine nucleotides. The purine nucleotides of the antisense region may be 2′-O-methyl purine nucleotides. The purine nucleotides present in the antisense region may comprise 2′-deoxy-purine nucleotides. The antisense region may comprise a phosphorothioate internucleotide linkage at the 3′ end of the antisense region. The antisense region may comprise a glyceryl modification at a 3′ end of the antisense region. Each of the two fragments of the siNA molecule may comprise about 21 nucleotides, and about 19 or 21 nucleotides of the antisense region may be base-paired to the nucleotide sequence of the IN RNA encoding the C-terminal domain of IN a portion thereof. Also, about 19 or 21 nucleotides of each fragment of the siNA molecule may be base-paired to the complementary nucleotides of the other fragment of the siNA molecule and at least two 3′ terminal nucleotides of each fragment of the siNA molecule may be not base-paired to the nucleotides of the other fragment of the siNA molecule. Each of the two 3′ terminal nucleotides of each fragment of the siNA molecule may be 2′-deoxy-pyrimidines which may be 2′-deoxy-thymidine. The 5′-end of the fragment comprising the antisense region may include a phosphate group.

A siNA specifically contemplated has RNAi activity against HIV-1 RNA. In one embodiment the siNA molecule comprises a sequence complementary to the RNA sequence set out in FIG. 23 and SEQ ID NOs 16-20.

(32) Compositions and Formulations

The IN-derived peptide, variant, fusions, antibodies, and nucleic acids of the invention may be used in compositions and formulations for treating conditions related to HIV-1 infection. The invention provides corresponding methods of treatment, in which a therapeutic dose of the IN-derived peptide, variant, fusion, antibodies, and nucleic acid is administered in a pharmacologically acceptable formulation, e.g. to a patient or subject in need thereof. Accordingly, the invention also provides therapeutic compositions comprising IN-derived peptide, variant, fusion, antibody, and nucleic acid, and a pharmacologically acceptable excipient or carrier. In one embodiment, such compositions include the IN-derived compounds in a therapeutically or prophylactically effective amount sufficient to treat a condition related to HIV-1 infection. The therapeutic composition may be soluble in an aqueous solution at a physiologically acceptable pH.

A “therapeutically effective amount” refers to an amount effective, at dosages and for periods of time necessary, to achieve the desired therapeutic result, such as a reduction of HIV-1 infection and in turn a reduction in disease progression. A therapeutically effective amount may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting the rate of disease onset or progression. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the IN-derived peptide, variant, fusion, antibody, and nucleic acid of the invention can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active IN-derived peptide, variant, fusion, antibody, and nucleic acid, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, a IN-derived peptide, variant, fusion, antibody, and nucleic acid may be formulated with one or more additional compounds that enhance the solubility of the IN-derived peptide, variant, fusion, antibody, and nucleic acid. In accordance with another aspect of the invention, therapeutic compositions of the present invention, comprising a IN-derived peptide, variant, fusion, antibody, and nucleic acid, may be provided in containers or commercial packages which further comprise instructions for use of the IN-derived peptide, variant, fusion, antibody, and nucleic acid for the inhibition of HIV-1 replication or infection.

Accordingly, the invention further provides a commercial package comprising a IN-derived peptide, variant, fusion, antibody, and nucleic acid or the above-mentioned composition together with instructions for the prevention and/or treatment of HIV-1-related disease.

The invention further provides a use of a IN-derived peptide, variant, fusion, antibody, and nucleic acid for inhibition of HIV-1 proliferation. The invention further provides a use of a IN-derived peptide, variant, fusion, antibody, and nucleic acid for the preparation of a medicament.

There are known in the art means to improve uptake of agents including peptides, proteins, and nucleic acids. One method utilizes agents that assist in cellular uptake such as chemicals that modify cellular permeability, liposomes for encapsulation of the agent. Neutral or anionic liposomes, microspheres, ISCOMS, or virus-like-particles (VLPs) are usually used to facilitate delivery of protein agents. These compounds are readily available to one skilled in the art; for example, see Liposomes: A Practical Approach, RCP New Ed, IRL press (1990). See also U.S. Pat. No. 6,417,326 herein incorporated by reference. In one recent method, agent uptake into the cell is via use of neutral liposomes conjugated with an arginine-rich membrane translocating peptide, e.g. from HIV-TAT, Antennapedia, and octaarginine (Cryan et al. Mol Pharm. 2006 March-April; 3(2):104-12).

(33) Screening Assays

In another aspect, the invention relates to the use of IN-derived peptides, variants and fusions, specifically the C-terminal domain of IN (residues 205-288) as a target in screening assays that may be used to identify compounds that are useful for inhibiting HIV-1 replication and infection. In some embodiments, such an assay may comprise the steps of

a) providing a test compound;
b) providing a source of the IN-derived peptides of the invention;
c) providing a source of imp7 or impβ; and
d) measuring the binding of the IN-derived peptides to imp7 or impβ in the presence versus the absence of the test compound. A lower measured binding in the presence of the test compound indicates that the compound is an inhibitor of the interaction and may be useful for the prevention and/or treatment of disease related to HIV-1 infection.

The assay methods of the invention may further be used to identify compounds capable of inhibiting HIV-1 proliferation in a biological system. Such an assay may further comprise the step of assaying the compounds for the reduction, abrogation or reversal of HIV-1 infection or persistence in an animal model.

The above-noted methods and assays may be employed either with a single test compound or a plurality or library (e.g. a combinatorial library) of test compounds. In the latter case, synergistic effects provided by combinations of compounds may also be identified and characterized. The above-mentioned compounds may be used as lead compounds for the development and testing of additional compounds having improved specificity, efficacy and/or pharmacological (e.g. pharmacokinetic) properties. In certain embodiments, one or a plurality of the steps of the screening/testing methods of the invention may be automated.

Such assay systems may comprise a variety of means to enable and optimize useful assay conditions. Such means may include but are not limited to: suitable buffer solutions, for example, for the control of pH and ionic strength and to provide any necessary components for optimal binding and stability, temperature control means for optimal binding and/or stability, and detection means to enable the detection of the binding. A variety of such detection means may be used, including but not limited to one or a combination of the following: radiolabelling (e.g. ³²P), antibody-based detection, fluorescence, chemiluminescence, spectroscopic methods (e.g. generation of a product with altered spectroscopic properties), various reporter enzymes or proteins (e.g. horseradish peroxidase, green fluorescent protein), specific binding reagents (e.g. biotin/(strept)avidin), and others. Binding may also be analysed using generally known methods in this area, such as electrophoresis on native polyacrylamide gels, as well as fusion protein-based assays such as the yeast 2-hybrid system or in vitro association assays, or proteomics-based approaches.

The assay may be carried out in vitro utilizing a source of IN-derived peptides, imp7 or impβ which may comprise naturally isolated or recombinantly produced IN-derived peptides, imp7 or impβ, in preparations ranging from crude to pure. Recombinant IN-derived peptides, imp7 or impβ may be produced in a number of prokaryotic or eukaryotic expression systems which are well known in the art. Such assays may be performed in an array format. In certain embodiments, one or a plurality of the assay steps are automated.

The assay may in an embodiment be performed using an appropriate host cell as a source of IN-derived peptides, imp7 or impβ. Such a host cell may be prepared by the introduction of DNA encoding the IN-derived peptides, imp7 or impβ into the host cell and providing conditions for the expression of the IN-derived peptides, imp7 or impβ. Such host cells may be prokaryotic or eukaryotic, bacterial, yeast, amphibian or mammalian.

The above-described assay methods may further comprise determining whether any compounds so identified can be used for the prevention or treatment of disease related to HIV infection, such as examining their effect(s) on disease symptoms in suitable disease animal model systems.

Specific screening methods are known in the art and along with integrated robotic systems and collections of chemical compounds/natural products are extensively incorporated in high throughput screening so that large numbers of test compounds can be processed within a short amount of time. These methods include homogeneous assay formats such as fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochermiluminescence, as well as more traditional heterogeneous assay formats such as enzyme-linked immunosorbant assays (ELISA) or radioimmunoassays. Also comprehended herein are cell-based assays, for example those utilizing reporter genes, as well as functional assays that analyze the effect of an antagonist or agonist on biological function(s).

Moreover, combinations of screening assays can be used to find molecules that affect the interaction between IN-derived peptides and imp7 or impβ. Molecules that regulate the biological activity of a polypeptide may be useful as agonists or antagonists of the peptide. In using combinations of various assays, it is usually first determined whether a candidate molecule binds to the IN-derived peptides, or to imp7 or impβ, by using an assay that is amenable to high throughput screening. Binding candidate molecules identified in this manner are then added to a biological assay to determine if there are effects on the biologically relevant interactions. Molecules that bind and that have an agonistic or antagonistic effect on biologic activity will be useful in treating or preventing disease or conditions with which the polypeptide(s) are implicated.

Homogeneous assays are mix-and-read style assays that are very amenable to robotic application, whereas heterogeneous assays require separation of free from bound analyte by more complex unit operations such as filtration, centrifugation or washing. These assays are utilized to detect a wide variety of specific biomolecular interactions (including protein-protein, receptor-ligand, enzyme-substrate, and so on), and the inhibition thereof by small organic molecules. These assay methods and techniques are well known in the art. The screening assays of the present invention are amenable to high throughput screening of chemical libraries and are suitable for the identification of small molecule drug candidates, antibodies, peptides, and other antagonists and/or agonists, natural or synthetic.

One such assay is based on fluorescence resonance energy transfer (FRET) between two fluorescent labels, an energy donating long-lived chelate label and a short-lived organic acceptor.

Another useful assay is BRET (Bioluminescence Resonance Energy Transfer). BRET is a protein-protein interaction assay based on energy transfer from a bioluminescent donor to a fluorescent acceptor protein. The BRET signal is measured by the amount of light emitted by the acceptor to the amount of light emitted by the donor. The ratio of these two values increases as the two proteins are brought into proximity. The BRET assay has been described in the literature. See, e.g., U.S. Pat. Nos. 6,020,192; 5,968,750; and 5,874,304; and Xu et al. (1999) Proc. Natl. Acad. Sci. USA 96:151-156. BRET assays may be performed by genetically fusing a bioluminescent donor protein and a fluorescent acceptor protein independently to two different biological partners to make partner A-bioluminescent donor and partner B-fluorescent acceptor fusions. Changes in the interaction between the partner portions of the fusion proteins, modulated, e.g., by ligands or test compounds, can be monitored by a change in the ratio of light emitted by the bioluminescent and fluorescent portions of the fusion proteins. A schematic depiction of the BRET assay, based on the 205-288 IN C-terminal domain for detecting interaction with impβ and imp7 in live cells as one example, is shown in FIG. 24.

Another assay that will be useful in the screening methods of the invention is FlashPlate (Packard Instrument Company, IL). This assay measures the ability of compounds to inhibit protein-protein interactions. FlashPlates are coated with a first protein (e.g. either imp7 or impβ), then washed to remove excess protein. For the assay, compounds to be tested are incubated with the second protein (e.g. the C-terminal domain of IN or the IN-derived peptides and fusions of the invention) and I¹²⁵ labeled antibody against the second protein are added to the plates. After suitable incubation and washing, the amount of radioactivity bound is measured using a scintillation counter.

Another useful assay is AlphaScreen, which an “Amplified Luminescent Proximity Homogeneous Assay” method utilizing latex microbeads (250 nm diameter) containing a photosensitizer (donor beads), or chemiluminescent groups and fluorescent acceptor molecules (acceptor beads). Upon illumination with laser light at 680 nm, the photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity to the donor bead (i.e., by virtue of the interaction of imp7 or impβ with the C-terminal domain of IN), the singlet-state oxygen molecules reacts with chemiluminescent groups in the acceptor beads, which immediately transfer energy to fluorescent acceptors in the same bead. These fluorescent acceptors shift the emission wavelength to 520-620 nm, resulting in a detectable signal. Antagonists of the interaction of imp7 or impβ with the C-terminal domain of IN will thus inhibit the shift in emission wavelength, whereas agonists of this interaction would enhance it.

All published documents mentioned in the above specification are herein incorporated by reference. Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Various modifications of the above-described modes for carrying out the invention which are clear to those skilled in the field of genetics and molecular biology or related fields are intended to be within the scope of the following claims.

We claim:

1. An isolated peptide comprising at least 8 and no more than 83 consecutive amino acids from residues 205 to 288 of an HIV-1 integrase sequence, wherein the at least 8 and no more than 83 consecutive amino acids of integrase comprises at least one of the following regions of SEQ ID NO:1: amino acids #211-219, amino acids #236-244, amino acids #235-250, amino acids #259-266, amino acids #258-266, and amino acids #261-268.

2. The peptide according to claim 1 wherein the at least 8 and no more than 83 consecutive amino acids of integrase comprises at least one of the following regions of SEQ ID NO:1: amino acids #210-225, amino acids #233-250, amino acids #261-273, amino acids #211-245, amino acids #236-266, and amino acids #211-266.

3. The peptide according to claim 1 wherein the at least 8 and no more than 83 consecutive amino acids of integrase comprises amino acids #211-219 of SEQ ID NO:1 or amino acids #236-244 of SEQ ID NO:1, or both.

4. The peptide according to claim 1 wherein the at least 8 and no more than 83 consecutive amino acids of integrase comprises amino acids #236-244 of SEQ ID NO:1 or amino acids #259-266 of SEQ ID NO:1 or both.

5. The peptide according to claim 1 wherein the at least 8 and no more than 83 consecutive amino acids of integrase comprises amino acids #211-219 of SEQ ID NO:1, amino acids #236-244 of SEQ ID NO:1, and amino acids #259-266 of SEQ ID NO:1.

6. The peptide according to claim 1 comprising at least 13 and no more than 83 consecutive amino acids from residues 205 to 288 of an HIV-1 integrase sequence, wherein the at least 13 and no more than 83 consecutive amino acids of integrase comprises at least one of the following regions of SEQ ID NO:1: amino acids #211-219, amino acids #236-244, amino acids #235-250, amino acids #259-266, amino acids #258-266, and amino acids #261-268.

7. The peptide according to claim 1 further comprising a heterologous sequence which is fused with the sequence derived from residues 205 to 288 of HIV-1 integrase.

8. The peptide according to claim 7 wherein the heterologous sequence is a membrane-translocating sequence.

9. The peptide according to claim 8 wherein the membrane-translocating sequence is the HIV Tat membrane-translocating sequence (SEQ ID NO:9).

10. The peptide according to claim 7 wherein the heterologous sequence is a reporter sequence.

11. The peptide according to claim 1 that, when expressed with HIV-1 provirus, renders HIV-1 replication-defective or infection-defective.

12. A variant polypeptide of HIV-1 integrase having a substitution or deletion in at least one of the following positions of HIV-1 integrase: K211, K215, K219, K236, K240, K244, V249, V250, K258, R262, R263, K264, K266, and K273.

13. A variant polypeptide of HIV-1 integrase having at least one of the following regions of SEQ ID NO:1 deleted: amino acids #211-219, amino acids #236-244, amino acids #235-250, amino acids #259-266, amino acids #258-266, and amino acids #261-268.

14. A fusion polypeptide comprising the variant polypeptide of claim 12 fused to a heterologous sequence.

15. An isolated polynucleotide encoding the peptide defined in claim 1.

16. An isolated polynucleotide encoding the variant polypeptide defined in claim 12.

17. A monoclonal antibody specifically immunoreactive against at least one of the following regions of SEQ ID NO:1: amino acids #211-219, amino acids #236-244, amino acids #235-250, amino acids #259-266, amino acids #258-266, amino acids #261-268, amino acids #210-225, amino acids #233-250, amino acids #261-273, amino acids #211-245, amino acids #236-266, and amino acids #211-266.

18. The monoclonal antibody according to claim 17 which is a single chain monoclonal antibody.

19. A chemically synthesized double stranded short interfering nucleic acid (siNA) molecule that directs cleavage via RNA interference (RNAi) of a HIV RNA encoding amino acids 205 to 288 of HIV-1 integrase, wherein a) each strand of said siNA molecule is about 18 to about 23 nucleotides in length; and b) one strand of said siNA molecule comprises nucleotide sequence having sufficient complementarity to said HIV RNA for the siNA molecule to direct cleavage of the HIV RNA via RNA interference.

20. A method of inhibiting HIV-1 replication in a cell, comprising transporting into the cell the peptide defined in claim 1.

21. A method of inhibiting HIV-1 replication in a cell, comprising expressing in the cell the polynucleotide defined in claim 15.

22. A method of inhibiting HIV-1 infection in a human comprising administering to the human the peptide defined in claim 1.

23. A method for screening for a compound that affects HIV-1 replication or infection, the method comprising: (a) incubating, in the presence of a candidate agent, the peptide defined in claim 1 with imp7 or impβ, under conditions suitable for binding to occur between the peptide and imp7 or impβ; (b) determining the level of binding between the peptide and imp7 or impβ, wherein detecting a change in the level of binding between the peptide and imp7 or impβ in the presence of the candidate agent, compared to the level of binding in the absence of the candidate agent, indicates that said agent is a compound that affects HIV-1 replication or infection.

24. A method for screening for a compound that affects HIV-1 replication or infection, the method comprising: (a) providing a cell that expresses (i) the peptide defined in claim 1 and (ii) imp7 or impβ; (b) providing the cell with a candidate agent; and (c) determining the level of binding between the expressed peptide and the expressed imp7 or impβ, wherein detecting a change in the level of binding between the peptide and imp7 or impβ in the presence of the candidate agent, compared to the level of binding in the absence of the candidate agent, indicates that said agent is a compound that affects HIV-1 replication or infection.

25. The method according to claim 23 for screening for a compound that inhibits HIV-1 replication or infection, and wherein detecting a decrease in the level of binding between the peptide and imp7 or impβ in the presence of the candidate agent, compared to the level of binding in the absence of the candidate agent, indicates that said agent is a compound that inhibits HIV-1 replication or infection.


SEQUENCE LISTING (35 sequences)

SEQ ID NO: 1 - protein, 288 aa, HIV- 1
FLDGIDKAQEDHEKYHSNWRAMASDFNLPPVVAKEIVASCDKCQLKGEAMHGQVDCSPGIWQLDCTHLEGKIILVAVHVASGYIEAEVIPAETGQETAYFILKLAGRWPVKTIHTDNGSNFTSTTVKAACWWAGIKQEFGIPYNPQSQGVVESMNKELKRIIGQVRDQAEHLKTAVQMAVLIHNFKRKGGIGGYTAGERIVDIIATDIQTKELQKQITKLQNFRVYYRDSRDPLWKGPAKLLWKGEGAVVIQDNSDIKVVPRRKAKIIRDYGKQMAGDDCVAGRQDED

SEQ ID NO: 2 - protein, 84 aa, HIV-1
ATDIQTKELQKQITKLQNFRVYYRDSRDPLWKGPAKLLWKGEGAVVIQDNSDIKVVPRRKAKIIRDYGKQMAGDDCVAGRQDED

SEQ ID NO: 3 - protein, 9 aa, HIV-1
KELQKQITK

SEQ ID NO: 4 - protein, 9 aa, HIV-1
KGPAKLLWK

SEQ ID NO: 5 - protein, 16 aa, HIV-1
WKGPAKLLWKGEGAVV

SEQ ID NO: 6 - protein, 8 aa, HIV-1
VVPRRKAK

SEQ ID NO: 7 - protein, 9 aa, HIV-1
KVVPRRKAK

SEQ ID NO: 8 - protein, 8 aa, HIV-1
PRRKAKII

SEQ ID NO: 9 - protein, 12 aa, HIV-1
AYGRKKRRQRRR

SEQ ID NO: 10 - protein, 16 aa, HIV-1
TKELQKQITKLQNFRV

SEQ ID NO: 11 - protein, 18 aa, HIV-1
PLWKGPAKLLWKGEGAVV

SEQ ID NO: 12 - protein, 13 aa, HIV-1
PRRKAKIIRDYGK

SEQ ID NO: 13 - protein, 35 aa, HIV-1
KELQKQITKLQNFRVYYRDSRDPLWKGPAKLLWKG

SEQ ID NO: 14 - protein, 31 aa, HIV-1
KGPAKLLWKGEGAVVIQDNSDIKVVPRRKAK

SEQ ID NO: 15 - protein, 56 aa, HIV-1
KELQKQITKLQNFRVYYRDSRDPLWKGPAKLLWKGEGAVVIQDNSDIKVVPRRKAK

SEQ ID NO: 16 - RNA, 174 nt, HIV-1
acuaaagaauuacaaaaacaaauuacaaaaauucaaaauuuucggguuuauuacagggacagcagagauccaguuuggaaaggaccagcaaagcuccucuggaaaggugaaggggcaguaguaauacaagauaauagugacauaaaaguagugccaagaagaaaagcaaagauc

SEQ ID NO: 17 - RNA, 21 nt, HIV-1
uuacaaaaacaaauuacaaaa

SEQ ID NO: 18 - RNA, 21 nt, HIV-1
uggaaaggaccagcaaagcuc

SEQ ID NO: 19 - RNA, 21 nt, HIV-1
gcaaagcuccucuggaaaggu

SEQ ID NO: 20 - RNA, 21 nt, HIV-1
gugccaagaagaaaagcaaag

SEQ ID NO: 21 - RNA, 25 nt, artificial
  imp7 primer 1
uaagcagauucccucaagcuguugg

SEQ ID NO: 22 - RNA, 26 nt, artificial
  imp7 primer 2
aaugcugcauugcuggcuaccaaugg

SEQ ID NO: 23 - DNA, 32 bp, artificial
  IN primer 1
gcgcaagcttggatagatgtttttagatggaa

SEQ ID NO: 24 - DNA, 24 bp, artificial
  IN primer 2
ccatgtgtggtacctcatcctgct

SEQ ID NO: 25 - DNA, 32 bp, artificial
  IN truncation primer 1
gcgcaagcttggatagatgcatggacaagtag

SEQ ID NO: 26 - DNA, 28 bp, artificial
  IN truncation primer 2
caattcccgggtttgtatgtctgtttgc

SEQ ID NO: 27 - DNA, 20 bp, artificial
  IN mutant primer 1
gcagctagcagggagactaa

SEQ ID NO: 28 - DNA, 27 bp, artificial
  IN mutant primer 2
ctgttcctgcagctaatcctcatcctg

SEQ ID NO: 29 - DNA, 23 bp, artificial
  RT primer 1
ggatggtgcttcaagctagtacc

SEQ ID NO: 30 - DNA, 27 bp, artificial
  RT primer 2
actgacgctctcgcacccatctctctc

SEQ ID NO: 31 - DNA, 27 bp, artificial
  Southern probe
ctctagcagtggcgcccgaacagggac

SEQ ID NO: 32 - DNA, 24 bp, artificial
  5′-Alu oligo
tcccagctactcgggaggctgagg

SEQ ID NO: 33 - DNA, 26 bp, artificial
  3′-LTR oligo
aggcaagctttattgagggcttaagc

SEQ ID NO: 34 - DNA, 21 bp, artificial
  Nested PCR primer 1
cacacacaaggctacttccct

SEQ ID NO: 35 - DNA, 20 bp, artificial
  Nested PCR primer 2
gccactccccagtcccgccc